(19) United States
(12) Patent Application Publication
(10) Pub. No.: US 2025/0002514 A1
(43) Pub. Date: Jan. 2, 2025

(54) AMORPHOUS FORM OF BISMUTH-1,2-ETHANEDITHIOL AND METHODS OF MAKING

(71) Applicant: Microbion Corporation, Bozeman, MT (US)

(72) Inventors: Jeffrey W. MILLARD, Bozeman, MT (US); Brett Hugh James BAKER, Bozeman, MT (US)

(21) Appl. No.: 18/273,841

(22) PCT Filed: Jan. 25, 2022
(86) PCT No.: PCT/US2022/013678

§ 371 Date: Jul. 24, 2023

Related U.S. Application Data
(60) Provisional application No. 63/141,081, filed on Jan. 25, 2021.

(51) Int. Cl. C07F 9/94; A61K 9/00; A61K 9/12; A61K 31/555; A61K 47/26; A61P 17/02; A61P 31/04

(52) U.S. Cl. CPC C07F 9/94; A61K 9/0014; A61K 9/0078; A61K 9/12; A61K 31/555; A61K 47/26; A61P 17/02; A61P 31/04

(57) ABSTRACT

The invention relates to an amorphous form of bismuth-1,2-ethanedithiol (BisEDT). Methods of making amorphous BisEDT and methods of use are also provided.

### CROSS-REFERENCE TO RELATED APPLICATIONS

This application claims the benefit of and priority to U.S. Provisional Application Ser. No. 63/141,081, filed Jan. 25, 2021, which is incorporated herein by reference in its entirety.

### BACKGROUND

Bismuth-1,2-ethanedithiol (BisEDT) has been previously reported as effective for the topical and/or local treatment of antibiotic-resistant and difficult to treat microbial infections. BisEDT has broad spectrum, antibacterial efficacy against a broad range of pathogens, including multiple priority pathogens identified by the US Centers for Disease Control and Prevention. In addition to antibacterial efficacy, BisEDT has also demonstrated the ability to eradicate microbial biofilms. This dual antimicrobial action from this first-in-class product provides a novel clinical approach to treating infections.

BisEDT and processes for its preparation are disclosed in International Patent Application Nos. PCT/US2010/023108, PCT/US2011/023549, PCT/US2011/047490, PCT/US2019/044489, and PCT/US2019/044495 which are hereby incorporated by reference in their entireties for all purposes, however, an amorphous form of BisEDT has never been previously synthesized despite reports to the contrary. For example, International Patent Application Nos. PCT/US2010/023108, PCT/US2011/023549, PCT/US2011/047490 described a synthesis of BisEDT under ethanolic solvent conditions, the product of which was believed to be amorphous; however, unexpectedly this form of BisEDT was discovered to be crystalline.

A new amorphous form of a compound may possess physical properties that differ from, and are advantageous over, those of other crystalline or amorphous forms. These include, packing properties such as molar volume, density and hygroscopicity; thermodynamic properties such as melting temperature, vapor pressure and solubility; kinetic properties such as dissolution rate and stability under various storage conditions; surface properties such as surface area, wettability, interfacial tension and shape; mechanical properties such as hardness, tensile strength, compactibility, handling, flow and blend; filtration properties; and improved efficacy and absorption. Variations in any one of these properties may affect the chemical and pharmaceutical processing of a compound as well as its bioavailability and may often render the new form advantageous for pharmaceutical and medical use.

There still remains an unmet need for amorphous forms of BisEDT having good physicochemical properties, desirable bioavailability, and advantageous pharmaceutical parameters. This invention meets those needs.

### SUMMARY

In some embodiments, the present disclosure provides an amorphous form of bismuth-1,2-ethanedithiol (BisEDT). In some embodiments, the X-ray powder diffraction pattern of amorphous BisEDT does not contain any distinct peaks. In some embodiments, the X-ray powder diffraction pattern of amorphous BisEDT is substantially similar to FIG. 1. In some embodiments, differential scanning calorimetry thermogram of amorphous BisEDT comprises an exothermic peak at about 168° C. In some embodiments, the differential scanning calorimetry thermogram further comprises an endotherm at about 64° C. and/or an endotherm peak at about 112° C. and/or an exotherm peak at about 145° C. In some embodiments, the differential scanning calorimetry thermogram is substantially similar to FIG. 2. In some embodiments, the amorphous BisEDT has a glass transition at about 101° C. In some embodiments, the amorphous form is at least 90% pure. For example, the amorphous form is at least 95% or 98% pure.

In some embodiments, the present disclosure provides a composition comprising an amorphous from of BisEDT. In some embodiments, the composition comprises at least one pharmaceutically acceptable carrier. In some embodiments, the composition comprises BisEDT in a suspension.

In some embodiments, the present disclosure provides a method of treating, managing or lessening the severity of symptoms and infections associated with one or more pulmonary diseases or infections in a subject, the method comprising administering to the subject a bismuth-thiol (BT) composition that comprises amorphous BisEDT suspended therein, wherein administering the BT composition is via inhalation, orally or nasally, using an aerosol device. In some embodiments, the method is treating, managing or lessening the severity of cystic fibrosis (CF) symptoms and infections in a subject.

In some embodiments, the present disclosure provides a method for healing a wound in a subject having a diabetic foot infection, comprising administering the subject a therapeutically effective amount of a composition comprising amorphous BisEDT. In some embodiments, the wound is a diabetic foot ulcer.

In some embodiments, the present disclosure provides a method of making an amorphous form of BisEDT, comprising (a) mixing an acidic aqueous solution that comprises a bismuth salt, with a solvent selected from the group consisting of acetone, acetonitrile, 1,2-dichloroethane, dimethyl sulfoxide, ethyl acetate, isopropanol, methyl tert-butyl ether, and mixtures thereof; (b) combing the product of (a) with a solution of 1,2-ethanedithiol in a solvent selected from the group consisting of acetone, acetonitrile, 1,2-dichloroethane, dimethyl sulfoxide, ethyl acetate, isopropanol, methyl tert-butyl ether, and mixtures thereof, under conditions and for a time sufficient for formation of a precipitate which comprises the amorphous form of BisEDT. In some embodiments, the method further comprises recovering the precipitate to remove impurities. In some embodiments, the bismuth salt is Bi(NO₃)₃. In some embodiments, 1,2-ethanedithiol is at a concentration of from about 1% wt/vol to about 20% wt/vol prior to step (b). In some embodiments, the acidic aqueous solution is prepared by mixing an aqueous suspension of either Bi (III) sub-nitrate or Bi (III) nitrate pentahydrate with an acid under conditions and for a time sufficient to form a substantially clear solution. In some embodiments, the concentration of either Bi (III) sub-nitrate or Bi (III) nitrate pentahydrate in the aqueous solution is from about 100 mg/mL to about 400 mg/mL. In some embodiments, the acid is 70% HNO₃. In some embodiments, the method further comprises adding the clear solution to an acidic solution. In some embodiments, the acidic solution is 5% HNO₃. In some embodiments, step (b) is performed at a temperature ranging from about 20° C. to about 28° C.

In some embodiments, the present disclosure provides a method of treating, managing or lessening the severity of cystic fibrosis (CF) symptoms and infections in a subject, the method comprising administering to the subject a bismuth-thiol (BT) composition that comprises amorphous BisEDT. In some embodiments, the BT composition comprises a plurality of microparticles wherein at least 70%, 80%, or 90% of said microparticles having a volumetric mean diameter (VMD) from about 0.01 μm to about 2.5 μm. In some embodiments, when the BT composition is aerosolized, at least 70%, 80%, or 90% of the aerosolized liquid droplets have a mass median aerodynamic diameter (MMAD) from about 0.03 μm to about 3 μm. In some embodiments, the BT composition comprises BisEDT at a concentration greater than about 0.1 mg/mL, about 0.05% to about 1.0% Tween 80®, about 40 mM to about 250 mM sodium chloride, and optionally about 2 to 20 mM sodium phosphate at about pH. 7.4. In some embodiments, the subject has at least one pulmonary infection containing one or more bacterial pathogens and/or fungal pathogens (as described herein). In some embodiments, the method comprises at least one of: (i) reducing a bacterial biofilm, (ii) impairing growth of a bacterial biofilm, (iii) preventing initial formation of the bacterial biofilm, and/or (iv) preventing reformation of the bacterial biofilm.

In some embodiments, the present disclosure provides an aerosol comprising a plurality of dispersed liquid droplets in a gas, said liquid droplets comprising a BT composition comprising amorphous BisEDT suspended therein; and wherein at least 70% of the liquid droplets have a MMAD from about 0.03 μm to about 3 μm. In some embodiments, prior to aerosolization, the BT composition comprises a plurality of microparticles wherein at least 70%, 80%, or 90% of said microparticles have a VMD from about 0.01 μm to about 2.5 μm. In some embodiments, the droplets further comprise Tween 80 and optionally a buffer at a pH of about 7.4; and/or sodium chloride.

In some embodiments, the present disclosure provides a pharmaceutical composition comprising bismuth-thiol (BT) composition that comprises amorphous BisEDT suspended therein, wherein the BT composition comprises a plurality of microparticles, wherein the D90 of said microparticles is less than or equal to 1.9 μm. In some embodiments, the BT composition comprises a plurality of microparticles, wherein the D90 of said microparticles is less than or equal to about 1.6 μm. In some embodiments, at least 70%, 80%, or 90% of said microparticles have a volumetric mean diameter from about 0.01 μm to about 2.5 μm.

In some embodiments, the present disclosure provides a method of treating, managing or lessening the severity of symptoms and infections associated with one or more pulmonary diseases or infections in a subject, the method comprising administering to the subject a bismuth-thiol (BT) composition that comprises amorphous BisEDT, wherein the BT composition comprises a plurality of microparticles wherein at least 70% of said microparticles having a volumetric mean diameter from about 0.01 μm to about 2.5 μm, and wherein when the BT composition is aerosolized, at least 70% of the aerosolized liquid droplets have a MMAD from about 0.03 μm to about 3 μm. In some embodiments, one or more pulmonary diseases or infections are not the result of or associated with cystic fibrosis. In some embodiments, the pulmonary infection is bronchiectasis infection, pneumonia, valley fever, allergic bronchopulmonary aspergillosis (ABPA), ventilator acquired pneumonia, hospital acquired pneumonia, community acquired pneumonia, ventilator associated tracheobronchitis, lower respiratory tract infection, non-tuberculous Mycobacteria (NTM), anthrax, legionellosis, pertussis, bronchitis, Bronchiolitis, COPD-associated infection, and post-lung transplantation. In some embodiments, the pulmonary infection is non-tuberculous Mycobacteria (NTM).

In some embodiments, the present disclosure provides a method for healing a wound in a subject having a diabetic foot infection, comprising administering the subject a therapeutically effective amount of a composition comprising amorphous BisEDT, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is healed or substantially healed within 12 weeks of the first administration of the composition. In some embodiments, the wound is a diabetic foot ulcer. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4. In some embodiments, the applied BT composition is present on the surface at a concentration from about 1 μg/cm² to about 1,000,000 μg/cm². In some embodiments, the applied BT composition is present on the surface at a concentration from about 50 μg/cm² to about 100 μg/cm². In some embodiments, the applied BT composition is present on the surface at a concentration greater than about 100 μg/cm². In some embodiments, the BT composition is administered three times per day, two times per day, once daily, every other day, once every three days, three times per week, once every week, once every other week, once every month, or once every other month. In some embodiments, the wound is healed 4 weeks, 8 weeks or 12 weeks after the first administration of the BT composition. In some embodiments, the subject is administered multiple doses of the BT composition daily or weekly for a length of time ranging from about one week to about 12 weeks.

In some embodiments, the subject is administered multiple doses of the BT composition daily or weekly for a length of about 4 weeks. In some embodiments, the wound area is from about 0.1 m² to about 250 cm².

In some embodiments, the present disclosure provides a method for wound size reduction in a subject having a diabetic foot infection, comprising administering to the subject a therapeutically effective amount of a composition comprising amorphous BisEDT, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is reduced in size from about a 1% reduction relative to the original wound size to total elimination of the wound within 12 weeks of the first administration of the composition. In some embodiments, the wound is reduced by about 10%, 20%, 30%, 40%, 50%, 60%, 70%, 80%, or 90%. In some embodiments, the wound is reduced by at least about 50%. In some embodiments, the wound is a diabetic foot ulcer. In some embodiments, the BT composition further comprises about 0.05% to about 1.0% Tween 80®, about 0.05 to 40 mM sodium chloride, optionally about 1% to about 10%/6 of methylcellulose, and optionally about 2 to 20 mM sodium phosphate at about pH. 7.4. In some embodiments, the applied BT composition is present on the surface at a concentration from about 1 μg/cm² to about 1,000,000 μg/cm². In some embodiments, the applied BT composition is present on the surface at a concentration from about 50 μg/cm² to about 100 μg/cm². In some embodiments, the applied BT composition is present on the surface at a concentration greater than about 100 μg/cm². In some embodiments, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of about 4 weeks. In some embodiments, the wound area is from about 0.1 cm² to about 250 cm². In some embodiments, the wound surface area of said wound is reduced by at least 50% by 12 weeks after the first administration of the BT composition. In some embodiments, the wound surface area of said wound is reduced by at least 50% by 4 weeks after the first administration of the BisEDT composition. In some embodiments, the wound surface area is measured using digital photographs or hand measurement.

In some embodiments, the present disclosure provides a pharmaceutical composition comprising bismuth-thiol (BT) composition that comprises amorphous BisEDT suspended therein, wherein the BT composition comprises a plurality of microparticles, wherein the D90 of said microparticles is less than or equal to 1.9 μm. In some embodiments, the BT composition comprises a plurality of microparticles, wherein the D90 of said microparticles is less than or equal to about 1.6 μm. In some embodiments, the present disclosure provides a method for healing a wound in a subject having a diabetic foot infection, comprising administering the subject a therapeutically effective amount of the composition.

### BRIEF DESCRIPTION OF THE DRAWINGS

FIG. 1 shows an XRPD pattern of amorphous BisEDT.

FIG. 2 shows a DSC thermogram for amorphous BisEDT.

FIG. 3 shows a temperature cycling DSC thermogram for amorphous BisEDT.

FIG. 4 shows a temperature modulated DSC thermogram for amorphous BisEDT.

FIG. 5 shows the effect of sputum on the bacterial killing activity of tobramycin.

FIG. 6 shows that the bactericidal activity of BisEDT appears to be partially inhibited by CF patient sputum

FIG. 7 shows that the bactericidal activity of BisBDT appears to be partially inhibited by CF patient sputum.

FIG. 8 shows a graph of lung tissue BisEDT concentration vs. time after a single 100 μg/kg lung deposited dose in rats.

FIG. 9 shows whole blood BisEDT concentration vs. time (100 μg/kg IV or 100 μg/kg inhalation or 250 μg/kg oral dose).

FIG. 10 shows rat blood BisEDT vs time after single inhalation dose (μg/kg lung deposited).

FIG. 11 shows rat lung BisEDT concentration (ng/g) at sacrifice (24 or 30 hours after single inhaled dose).

FIG. 12 shows Particle Size Distribution for vehicle.

FIG. 13 shows Particle Size Distribution for Tobramycin.

FIG. 14 shows Particle Size Distribution for BisEDT.

FIG. 15 shows rat efficacy figures showing cumulative (total) administered dose (lung deposited) at days 3 and 5.

### DETAILED DESCRIPTION

### Definitions

Unless defined otherwise, all technical and scientific terms used herein have the meaning commonly understood by a person skilled in the art of the present disclosure. The following references provide one of skill with a general definition of many of the terms used in this disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the verb “comprise” as is used in this description and in the claims and its conjugations are used in its non-limiting sense to mean that items following the word are included, but items not specifically mentioned are not excluded. The present disclosure may suitably “comprise”, “consist of”, or “consist essentially of”, the steps, elements, and/or reagents described in the claims.

Unless specifically stated or obvious from context, as used herein, the term “or” is understood to be inclusive. Unless specifically stated or obvious from context, as used herein, the terms “a”, “an”, and “the” are understood to be singular or plural.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

The term “bismuth” refers to the 83^{rd} element of the periodic table, or atoms or ions thereof. Bismuth can occur in the metallic state or in the ionized state, such as in the III or V oxidation state. Bismuth ions can form complexes with anions, either to make bismuth salts, or to form complex anions which are then further complexed with one or more additional cation(s). Bismuth can also form covalent bonds to other atoms, such as sulfur.

As disclosed herein, a “bismuth-thiol compound” or “BT compound” is a compound that has a bismuth atom covalently bound to one, two or three other sulfur atoms present on one or more thiol compounds. The term “thiol” refers to a carbon-containing compound, or fragment thereof, containing an —SH group and can be represented by the general formula R-SH. These thiol compounds include compounds with one, two, three or more S atoms. Thiol compounds can have other functionality, such as alkyl, hydroxyl, carbocyclyl, heterocyclyl, aryl, heteroaryl, amino, and other substituents. Thiol compounds having two or more S atoms can chelate the bismuth atom, such that two S atoms from the same molecule covalently bond with the bismuth atom. BisEDT has the following structure:

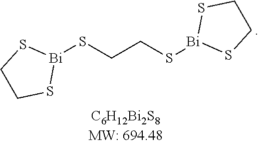

The term “amorphous” can be used in the context of this invention to designate the state of solid substances, in which the components (which can be atoms, ions or molecules), do not exhibit any periodic arrangement over a great range (=long-range order). For example, in the case of amorphous BisEDT, the BisEDT molecules or ions do not exhibit any periodic arrangement over a great range (=long-range order). In amorphous substances, the components are usually not arranged in a totally disordered fashion and completely randomly, but are rather distributed in such a way that a certain regularity and similarity to the crystalline state can be observed with regard to the distance from and orientation towards their closest neighbours (=short-range order). Amorphous substances consequently preferably possess a short-range order, but no long-range order as present in a crystal lattice. The identification and characterization of various morphic or amorphic forms of a pharmaceutically active compound is of great significance in obtaining medicaments with desired properties including a specific dissolution rate, milling property, bulk density, thermal stability or shelf-life.

The term “glass transition temperature” (Tg) is reported to describe the temperature at which amorphous or partially crystalline polymers change from the rigid solid state to the rubbery or more flexible state. In the process, a distinct change in physical parameters, e.g. hardness and elasticity, occurs. Beneath the Tg, a polymer is usually glassy and hard, whereas above the Tg, it changes into a rubber-like to viscous state.

The term “impurity” of a compound, as used herein, means chemicals other than the compound, including, derivatives of the compound, or degradants of the compound, or incompletely reacted reagents of the synthesis that remain with the product compound due to incomplete purification, or that develop over time, such as during stability testing, formulation development of the compound or storage of the compound.

The term “chemical purity” of a compound, as used herein, refers to the purity of a compound from other distinct chemical entities. For example, amorphous BisEDT having 90% chemical purity means that the amorphous form contains less than 10% of molecules or chemical entity different from BisEDTm including synthetic byproducts, residual solvents, or residual organic or inorganic substances.

The term “substantially similar” as used herein with regards to an analytical spectrum, such as XRPD patterns, Raman spectroscopy, etc., means that a spectrum resembles the reference spectrum to a great degree in both the peak locations and their intensity.

The term “substantially free of” as used herein, means free from therapeutically effective amounts of compounds when administered in suggested doses, but may include trace amounts of compounds in non-therapeutically effective amounts.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week.

“Coadministration” refers to the administration of the two agents in any manner in which the pharmacological effects of both agents are manifest in the patient at the same time. Thus, concomitant administration does not require that a single pharmaceutical composition, the same dosage form, or even the same route of administration be used for administration of both agents or that the two agents be administered at precisely the same time. However, in some situations, coadministration will be accomplished most conveniently by the same dosage form and the same route of administration, at substantially the same time.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “managing” includes therapeutic treatments as defined above. Managing includes achieving a steady state level of infection as determined by known methods in the art. The steady state can include evaluation of one or more of the severity of the infection(s), the size and location of the infection(s), the number of different microbial pathogens present in the infection(s), the level of antibiotic tolerant or resistant microbial pathogens, the degree of response to treatment, such as with a BT composition disclosed herein, the degree of biofilm formation and reduction, and the side effects experienced by the subject. During management of an infection, the infection may fluctuate from increasing to lessening in severity, in the amount or extent of infection, amount of side effects experienced by the subject, or other subject outcome indicia. Over a period of time, such as days, month, or years, the degree of management of the infection can be determined by evaluation of the above factors to assess whether the clinical course of infection has improved, is bacteriostatic, or has worsened. In some embodiments, managing an infection include successful treatment of microbial pathogen(s) that are otherwise drug tolerant or drug resistant.

The term “lessen the severity” of infection(s) refers to an improvement in the clinical course of the infection on any measurable basis. Such basis can include measurable indices such as reducing the extent of infection (s), whether the infection(s) are considered acute, the number and identity of microbial pathogens causing the infection(s), the extent of microbial (e.g. bacterial or fungal) biofilms, and side effects experienced by the subject. In some embodiments, lessening the severity of an infection is determined by measurements such as reduction in sputum infection counts (e.g. a reduction in CFU in the sputum). In some embodiments, lessening the severity involves halting a steady decline in outcome to achieve stabilized infection(s), resulting in the subject entering successful management of the infection(s). In other embodiments, lessening the severity can result in substantial to complete treatment of the infection(s). In some embodiments, lessening the severity refers to a lessening of exacerbations associated with the disease or infection (for example by a 1%-99% decrease in exacerbations). In some embodiments, lessening the severity can refer to an increase in lung function (for example by a 1%-99%/o increase in lung function).

The term “infection” is used herein in its broadest sense and refers to any infection, such as viral infection or caused by a microorganism bacterial infection, fungal infection or parasitic infection (e.g. protozoa, amoeba or helminths). Examples of such infections can be found in a number of well-known texts such as “Medical Microbiology” (Greenwood, D., Slack, R., Peutherer, J., Churchill Livingstone Press, 2002); “Mims' Pathogenesis of Infectious Disease” (Mims, C., Nash, A., Stephen, J., Academic Press, 2000); “Fields” Virology. (Fields, B N, Knipe D M, Howley, P M, Lippincott Williams and Wilkins, 2001); and “The Sanford Guide To Antimicrobial Therapy,” 26th Edition, JP Sanford et al. (Antimicrobial Therapy, Inc., 1996), which is incorporated by reference herein. The presence of infection in e.g. a diabetic foot wound is defined by clinical signs and symptoms of infection or inflammation, not by the culture of microorganisms, which are always present. However, immediately following resolution of clinical signs and symptoms of a wound infection, most patients will still have the underlying ulcer (e.g. diabetic foot ulcer), which requires continued treatment to facilitate complete wound closure. Of note, however, is that many wound specialists believe that in addition to the clinically defined state of infection, a less clinically apparent pathological state, known as “critical colonization” exists. In this state, a wound may be delayed or arrested in wound healing due to the subclinical presence of a high level of bacteria. This critical colonization, sometimes referred to as a high ‘wound bioburden’, is often polymicrobial and associated with biofilm-producing bacteria; it has been shown to induce, or prolong, the active inflammatory phase of repair, thus preventing a normal wound healing process. The bacterial cells that comprise such biofilms are difficult to recognize because they often exist in a viable, but nonculturable (VBNC), state (Pasquaroli 2013), yet they are adherent to surfaces and are typically more tolerant and resistant than their planktonic counterparts to antibiotics and antiseptics (Costerton 1999, Nguyen 2011). The term “infection” therefore contemplates the clinically defined state of infection as well as “critical colonization.”

The term “wound closure” can encompass healing of a wound wherein sides of the wound are rejoined to form a continuous barrier (e.g., intact skin). In another embodiment, the compositions and methods provided herein promote tissue regeneration. In another embodiment, the compositions and methods provided herein limit scarring of tissues such as glia, tendons, eye tissue, ligament or skin. In some embodiments, “wound closure” refers to complete or substantially complete re-epithelialization. In some embodiments, “wound closure” occurs via secondary intention.

It is to be understood that the term “wound healing” can encompass a regenerative process with the induction of a temporal and spatial healing program comprising wound closure and the processes involved in wound closure. The term “wound healing” can also encompass the processes of granulation, neovascularization, fibroblast, endothelial and epithelial cell migration, extracellular matrix deposition, re-epithelialization, and remodeling. In some embodiments, “wound healing” refers to a wound remaining closed for a sufficient period of time after the initial wound closure (e.g. one day, two days, three days, four days, five days, six days, one week, two weeks, three weeks, or one month). In some embodiments, “wound healing” refers to a wound remaining closed for two weeks after the initial wound closure.

It will be appreciated by a skilled artisan that the term “granulation” can encompass the process whereby small, red, grainlike prominences form on a raw surface (that of wounds or ulcers) as healing agents. Granulation may also include the formation of granulation tissue over the wound. “Granulation tissue” refers to the newly growing tissue material at a wound site formed to heal the wound. The tissue is perfused, fibrous connective tissue including a variety of cell types. The tissue will grow generally from the base of the wound to gradually fill the entire wound space.

It will be appreciated by a skilled artisan that the term “neovascularization” can encompass the new growth of blood vessels with the result that the oxygen and nutrient supply is improved. Similarly, it will be appreciated by the skilled artisan that the term “angiogenesis” may encompass the vascularization process involving the development of new capillary blood vessels. It will also be appreciated that the term “cell migration” refers to the movement of cells (e.g., fibroblast, endothelial, epithelial, etc.) to the wound site.

It is to be understood that the term “extracellular matrix deposition” can encompass the secretion by cells of fibrous elements (e.g., collagen, elastin, reticulin), link proteins (e.g., fibronectin, laminin), and space filling molecules (e.g., glycosaminoglycans). It will be appreciated by the skilled artisan that the term “type I collagen” can encompass the most abundant collagen, which forms large well-organized fibrils having high tensile strength.

It will be appreciated by a skilled artisan that the term “re-epithelialization” can encompass the reformation of epithelium over a denuded surface (e.g., wound).

The term “remodeling” refers to the replacement of and/or devascularization of granulation tissue.

As used herein, the term “exacerbation” refers to an increase in the severity of symptoms during a course of a disease which is mostly associated with a worsening of quality of life. Exacerbations are quite frequent in patients with chronic lung diseases such as CF. By definition, exacerbations are simply a worsening and/or increase in symptoms.

In some embodiments, lessening the severity of infections and/or symptoms can relate to patient-reported outcomes (“PROSs”). A PRO instrument is defined as any measure of a subject's health status that is elicited from the patient and determines how the patient “feels or functions with respect to his or her health condition.” PROs are particularly useful in reporting outcomes in CF and whether the severity of symptoms has been reduced or lessened. Such symptoms can be observable events, behaviors, or feelings (e.g., ability to walk quickly, lack of appetite, expressions of anger), or unobservable outcomes that are known only to the patient (e.g., perceptions of pain, feelings of depression). PROs are also particularly useful in reporting outcomes in DFI and whether the severity of symptoms has been reduced or lessened. Such symptoms can be observable events, behaviors, or feelings (e.g., ability to walk quickly, lack of appetite, expressions of anger), or unobservable outcomes that are known only to the patient (e.g., perceptions of pain, feelings of depression). In some embodiments, lessening the severity of infections and/or symptoms can be determined by physician assessments commonly known in the art, for example by an 8 item wound score.

An “effective amount”, as used herein, refers to an amount that is sufficient to achieve a desired biological effect. A “therapeutically effective amount”, as used herein refers to an amount that is sufficient to achieve a desired therapeutic effect. For example, a therapeutically effective amount can refer to an amount that is sufficient to improve at least one sign or symptom of infection (e.g. respiratory infection).

A “response” to a method of treatment can include a decrease in or amelioration of negative symptoms, a decrease in the progression of an infection or symptoms thereof, an increase in beneficial symptoms or clinical outcomes, a lessening of side effects, stabilization of the infection, and partial or complete remedy of infection, among others.

“Antibiotic susceptibility or sensitivity” refers to whether a bacteria will be successfully treated by a given antibiotic. Similarly, “Antifungal susceptibility or sensitivity” refers to whether a fungi will be successfully treated by a given antibiotic. Testing for susceptibility can be performed by methods known in the art such as the Kirby-Bauer method, the Stokes method and Agar Broth dilution methods. The effectiveness of an antibiotic in killing the bacteria or preventing bacteria from multiplying can be observed as areas of reduced or stable amount, respectively, of bacterial growth on a medium such as a wafer, agar, or broth culture.

“Antimicrobial tolerance” refers to the ability of a microbe, such as bacteria or fungi, to naturally resist being killed by antibiotics. It is not caused by mutant microbes but rather by microbial cells that exist in a transient, dormant, non-dividing state. Antibiotic or drug tolerance is caused by a small subpopulation of microbial cells termed persisters. Persisters are not mutants, but rather are dormant cells that can survive the antimicrobial treatments that kill the majority of their genetically identical siblings. Persister cells have entered a non- or extremely slow-growing physiological state which makes them insensitive (refractory or tolerant) to the action of antimicrobial drugs. Similarly, “antibiotic tolerance” refers to the ability of a bacteria to naturally resist being killed by antibiotics and “antifungal tolerance” refers to the ability of a fungi to naturally resist being killed by antibiotics.

“Antimicrobial resistance” refers to the ability of a microbe to resist the effects of medication that once could successfully treat the microbe. Microbes resistant to multiple antimicrobials are called multidrug resistant (MDR). Resistance arises through one of three mechanisms: natural resistance in certain types of bacteria, genetic mutation, or by one species acquiring resistance from another. Mutations can lead to drug inactivation, alteration of the drug's binding site, alteration of metabolic pathways and decreasing drug permeability.

As used herein, the terms “antibacterial activity”, “antifungal activity” and “antimicrobial activity”, with reference to a BT composition of the present disclosure, refers to the ability to kill and/or inhibit the growth or reproduction of a particular microorganism. In certain embodiments, antibacterial or antimicrobial activity is assessed by culturing bacteria, e.g., Gram-positive bacteria (e.g., S. aureus), Gram-negative bacteria (e.g., A. baumannii, E. coli, and/or P. aeruginosa) or bacteria not classified as either Gram-positive or Gram-negative, or fungi according to standard techniques (e.g., in liquid culture or on agar plates), contacting the culture with a BT composition of the present disclosure and monitoring cell growth after said contacting. For example, in a liquid culture, bacteria may be grown to an optical density (“OD”) representative of a mid-point in exponential growth of the culture; the culture is exposed to one or more concentrations of one or more BT compounds of the present disclosure, or variants thereof, and the OD is monitored relative to a control culture. Decreased OD relative to a control culture is representative of antibacterial activity (e.g., exhibits lytic killing activity). Similarly, bacterial colonies can be allowed to form on an agar plate, the plate exposed to a BT composition of the present disclosure, or variants thereof, and subsequent growth of the colonies evaluated related to control plates. Decreased size of colonies, or decreased total numbers of colonies, indicate antibacterial activity.

“Biofilm” refers any syntrophic consortium of microorganisms in which cells stick to each other and often also to a surface. These adherent cells become embedded within a slimy extracellular matrix that is composed of extracellular polymeric substances (EPS). Upon formation of biofilms, microbial resistance to antibiotics is up to 1000 times greater compared to that of planktonic bacteria. Bacterial aggregates are clusters of laterally aligned cells can initiate biofilm development, which has a more complex and denser 3-D structure. In some embodiments, the biofilm may comprise one or more species of bacteria (e.g., Pseudomonas aeruginosa and Staphylococcus aureus) and/or one or more different phyla (e.g., bacteria, virus and fungi).

As used herein, discussion of bacterial or fungal pathogens also encompass any microbe (bacteria and/or fungi) that contributes to the pathological state in the lungs. This includes both recognized and unrecognized microbes, and may also include bacteria or fungi that are not pathogens, but that simply facilitate the activity and presence of pathogens and their biofilms. As an example, embodiments directed to the inhibition of cell viability or cell growth of planktonic cells of the bacterial or fungal pathogen also extend to the inhibition of cell viability or cell growth of planktonic cells of the bacterial and/or fungal microbes that simply facilitate the activity and presence of pathogens and their biofilms.

“Airway surface” and “pulmonary surface,” as used herein, include pulmonary airway surfaces such as the bronchi and bronchioles, alveolar surfaces, and nasal and sinus surfaces.

“Saline” as used herein refers to a solution comprised of, consisting of, or consisting essentially of sodium chloride in water. Saline can be hypertonic, isotonic, or hypotonic. In some embodiments, saline can comprise sodium chloride in an amount of from about 0.1% to about 40% by weight, or any range therein, such as, but not limited to, about 0.1% to about 10%, about 0.5% to about 15%, about 1% to about 20%, about 5% to about 25%, about 10% to about 40%, or about 15% to about 35% by weight (in mg/100 mL). In certain embodiments, sodium chloride is included in a solution in an amount of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% by weight (in mg/100 mL), or any range therein.

“Hypertonic saline” as used herein refers to a solution comprised of, consisting of, or consisting essentially of greater than 0.9 wt % sodium chloride in water. In general, the sodium chloride is included in the solution in an amount of from about 0.9% to about 40% by weight, or any range therein, such as, but not limited to, about 1% to about 15%, about 5% to about 20%, about 5% to about 25%, about 10% to about 40%, or about 15% to about 35% by weight. In certain embodiments, sodium chloride is included in the solution in an amount of about 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% by weight, or any range therein.

“Hypotonic saline” as used herein refers to a solution comprised of, consisting of, or consisting essentially of less than 0.9 wt % sodium chloride in water. In some embodiments, sodium chloride is included in the solution in an amount of about 0.9%, 0.8%, 0.7%, 0.6%, 0.5%, 0.4%, 0.3%, 0.2%, 0.1% by weight, or any range therein.

“Isotonic saline” as used herein refers to a solution comprised of, consisting of, or consisting essentially of 0.9 wt % sodium chloride in water.

According to some embodiments, saline (e.g., hypertonic saline) can include an excipient. An excipient can be a pharmaceutically acceptable excipient. “Pharmaceutically acceptable” as used herein means that the compound or composition is suitable for administration to a subject to achieve the treatments described herein, without unduly deleterious side effects in light of the severity of the disease and necessity of the treatment. Exemplary excipients include, but are not limited to, a buffer and/or a buffering agent (e.g., an anion, a cation, an organic compound, a salt, etc.). Exemplary buffers include, but are not limited to, carbonic acid/carbonate/bicarbonate-based buffers, disodium hydrogen phthalate/sodium dihydrogen orthophosphate-based buffers, tris(hydroxymethyl)aminomethane/hydrochloric acid-based buffers, barbitone sodium/hydrochloric acid-based buffers, and any combination thereof. Exemplary buffering agents include, but are not limited to, carbonic acid, carbonate, bicarbonate, disodium hydrogen phthalate, sodium dihydrogen orthophosphate, tris(hydroxymethyl)aminomethane, hydrochloric acid, barbitone sodium, dissolved CO₂ (e.g., CO₂ formulated at a pH of greater than 6.6), and any combination thereof. In certain embodiments, saline comprises a bicarbonate buffer excipient, such as a bicarbonate anion (HCO₃). In some embodiments, hypertonic saline can include sodium bicarbonate, sodium carbonate, carbonic acid, and/or dissolved CO₂ formulated at a pH of greater than 6.5. Additional ingredients can be included as desired depending upon the particular condition being treated, as discussed further below.

As used herein, the term “volumetric median diameter” or “VMD” of an aerosol is the particle size diameter identified such that half of the mass of the aerosol particles is contained in particles with larger diameter than the VMD, and half of the mass of the aerosol particles is contained in particles with smaller diameter than the VMD. VMD is typically measured by laser diffraction.

“Mass median aerodynamic diameter” or “MMAD” is a measure of the aerodynamic size of a dispersed aerosol particle. The aerodynamic diameter is used to describe an aerosolized particle in terms of its settling behavior, and is the diameter of a unit density sphere having the same settling velocity, generally in air, as the particle in question. The aerodynamic diameter encompasses particle shape, density and physical size of a particle. As used herein, MMAD refers to the midpoint or median of the aerodynamic particle size distribution of an aerosolized particle determined by cascade impaction and/or laser time of flight and/or cascade impactor.

“Mass median diameter” or “MMD” is a measure of mean particle size. Any number of commonly employed techniques can be used for measuring mean particle size.

As used herein, “D90” refers to the 90% value of particle diameter (either the microparticle or aerosolized particle). For example if D90=1 μm, 90% of the particles are smaller than 1 μm. Similarly, “D80” refers to the 80% value of particle diameter (either the microparticle or aerosolized particle), “D70” refers to the 70% value of particle diameter (either the microparticle or aerosolized particle), “D60” refers to the 60% value of particle diameter (either the microparticle or aerosolized particle), “D50” refers to the 50% value of particle diameter (either the microparticle or aerosolized particle), “D40” refers to the 40% value of particle diameter (either the microparticle or aerosolized particle), “D30” refers to the 30% value of particle diameter (either the microparticle or aerosolized particle), “D20” refers to the 20% value of particle diameter (either the microparticle or aerosolized particle), “D10” refers to the 10% value of particle diameter (either the microparticle or aerosolized particle).

As used herein, “monodisperse” refers to a collection of particles (bulk or aerosol dispersion) comprising particles of a substantially uniform MMD and/or MMAD and/or VMD.

As used herein, the term “deposition efficiency” refers to the percentage of the delivered dose that is deposited into the area of interest. Thus, the deposition efficiency of a method and/or system for delivering an aerosolized medicament into the lungs is the amount by mass of the aerosol deposited into the lungs divided by the total amount of the aerosol delivered by the system to the nares and/or mouth.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

### Amorphous BisEDT

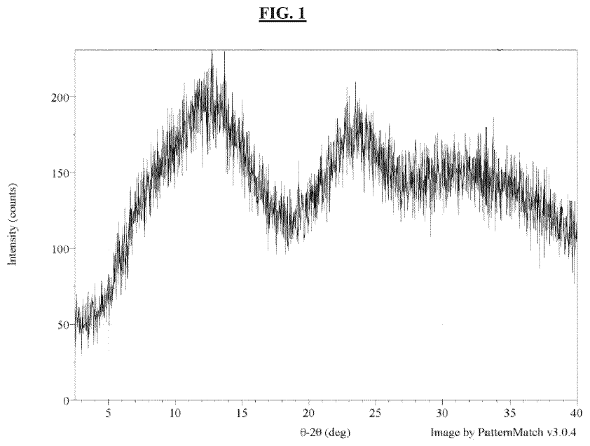

In some embodiments, the present disclosure provides an amorphous form of bismuth-1,2-ethanedithiol (BisEDT). The preferred method of differentiating amorphous BisEDT from crystalline and other non-crystalline forms of BisEDT is X-ray powder diffraction (XRPD). The spectrum of XRPD is typically represented by a diagram plotting the intensity of the peaks versus the location of the peaks, i.e., diffraction angle 20 (two-theta) in degrees. The XRPD pattern of pure amorphous BisEDT, as illustrated in FIG. 1, can be seen to lack discernible acute peaks. Thus, amorphous BisEDT, according to the present invention, is characterized in providing an X-ray powder diffraction pattern containing one or more broad diffuse halos having very low counts (i.e. see FIG. 1) in contrast to the sharp diffraction peaks characteristic of crystalline materials. The term “broad diffuse halo” is the art recognized term for the ‘humps’ observed in XRPD (see Klug and Alexander, X-ray diffraction procedures: for polycrystalline and amorphous materials, 2nd edition, 1974, John Wiley and Sons, pp 79 1-792). Of course it will be appreciated that a mixture comprising detectable amounts of both crystalline and amorphous BisEDT will exhibit both the characteristic sharp peaks and the diffuse halo(s) on XRPD. This will be evident by an increase in the baseline/background intensities and also a reduction in crystalline peak intensities when compared to the pure crystalline form's XRPD diffractogram. Accordingly, in some embodiments, the X-ray powder diffraction pattern of amorphous BisEDT does not contain any distinct peaks. In some embodiments, the X-ray powder diffraction pattern of amorphous BisEDT is substantially similar to FIG. 1.

In some embodiments, the amorphous form is stable. The term stable as used herein, refers to the tendency to remain substantially in the same physical form for at least a month, preferably at least 6 months, more preferably at least a year, still more preferably at least 3 years, even still more preferably at least 5 years, when stored under ambient conditions (25° C./60% RH) without external treatment. As noted above amorphous forms of many compounds often revert to the crystalline form in a relatively short time period (days/weeks rather than months/years). Substantially the same physical form in this context means that at least 70%, preferably at least 80% and more preferably at least 90% of the amorphous form remains.

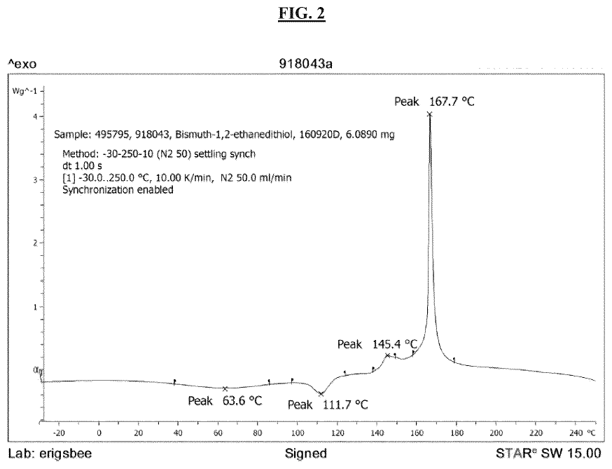

In one embodiment, the amorphous BisEDT is characterized by Differential Scanning Calorimetry (DSC). The DSC thermogram is typically expressed by a diagram plotting the normalized heat flow in units of Watts/gram (“W/g”) versus the measured sample temperature in degree C. The DSC thermogram is usually evaluated for extrapolated onset and end (outset) temperatures, peak temperature, and heat of fusion. In some embodiments, the differential scanning calorimetry thermogram of amorphous BisEDT comprises an exothermic peak at about 168° C., likely related to decomposition. In some embodiments, the differential scanning calorimetry thermogram further comprises an endotherm at about 64° C. and/or an endotherm peak at about 112° C. and/or an exotherm peak at about 145° C. In some embodiments, the differential scanning calorimetry thermogram is substantially similar to FIG. 2.

In some embodiment, amorphous BisEDT has a glass transition point (Tg), measured by Differential Scanning Calorimetry (DSC), modulated DSC, or Thermal Mechanical Analysis (TMA), greater than about 70° C., about 80° C., about 90° C., about 95° C., or 100° C. It is generally regarded that as a rough rule of thumb a Tg of 50° C. or greater above the storage temperature should assure reasonable physical stability to crystallization. Accordingly, in some embodiments the amorphous BisEDT has, when dry, a glass transition point (Tg) measured by Differential Scanning Calorimetry (DSC), modulated DSC, or Thermal Mechanical Analysis (TMA) of 100° C. or more. in some embodiments the amorphous BisEDT has, when dry, a glass transition point (Tg) measured by modulated DSC of 100° C. or more. In some embodiments, the amorphous BisEDT has a glass transition at about 101° C. In some embodiments, the amorphous BisEDT has a glass transition at about 101° C. as measured by modulated DSC.

Thus, according to further embodiments, there is provided amorphous BisEDT, providing an X-ray powder diffraction pattern containing one or more broad diffuse halos having low counts, and possessing, when dry, a glass transition point (Tg) of about 101° C. In some embodiments, the present disclosure provides amorphous BisEDT, providing an X-ray powder diffraction pattern containing one or more broad diffuse halos having low counts, and possessing, when dry, a glass transition point (Tg) measured by modulated DSC of at about 101° C. In some embodiments, the present disclosure provides amorphous BisEDT, providing an X-ray powder diffraction pattern substantially similar to FIG. 1, and possessing, when dry, a glass transition point (Tg) measured by modulated DSC of at about 101° C. In some embodiments, the present disclosure provides amorphous BisEDT, providing an X-ray powder diffraction pattern substantially similar to FIG. 1, and possessing, when dry, a glass transition point (Tg) of at about 101° C.

In some embodiments, the amorphous BisEDT form is at least 60% pure, at least, 70% pure, at least 80% pure, at least 90% pure at least 95% pure, at least 98% pure, or at least 99% pure. For example, the amorphous form in a composition is 60% pure, at least, 70% pure, at least 80% pure, at least 90% pure at least 95% pure, at least 98% pure, or at least 99% pure. In a specific embodiment, the composition is a pharmaceutical composition. Amorphous BisEDT, or the presence of some amorphous BisEDT, can be distinguished from crystalline BisEDT, using a variety of means, including but not limited to X-ray powder diffraction, Raman spectroscopy, solution calorimetry, differential scanning calorimetry, solid state nuclear magnetic resonance spectra (ssNMR) or infra-red spectroscopy. Each of these techniques is well established in the art. Amorphous BisEDT can also be identified based on the morphology of the particles seen under an electron microscope. Furthermore, amorphous BisEDT may be much more soluble than crystalline BisEDT, providing another means of discriminating between the crystalline and amorphous BisEDT forms, or detecting an amount of amorphous form within a BisEDT preparation.

In some embodiments, the amorphous BisEDT of the present invention is substantially free from other forms of BisEDT. Substantially free from other forms of BisEDT shall be understood to mean that amorphous BisEDT contains less than 50%, preferably less than 25%, more preferably less than 10% and still more preferably less than 5% of any other forms of BisEDT, e.g. crystalline BisEDT.

### Methods of Making

In some embodiments, the present disclosure provides a method of making an amorphous form of BisEDT, comprising (a) mixing an acidic aqueous solution that comprises a bismuth salt, with a solvent selected from the group consisting of acetone, acetonitrile, 1,2-dichloroethane, dimethyl sulfoxide, ethyl acetate, isopropanol, methyl tert-butyl ether, and mixtures thereof; (b) combing the product of (a) with a solution of 1,2-ethanedithiol in a solvent selected from the group consisting of acetone, acetonitrile, 1,2-dichloroethane, dimethyl sulfoxide, ethyl acetate, isopropanol, methyl tert-butyl ether, and mixtures thereof, under conditions and for a time sufficient for formation of a precipitate which comprises the amorphous form of BisEDT. No amorphous forms of BisEDT are known or disclosed in the prior art. The previously reported syntheses of BisEDT all were run under various conditions, such as ethanolic solvent conditions that produced crystalline BisEDT. While ethanol and a variety of other common solvents produced crystalline forms of BisEDT, the use of other solvents, such as acetone, acetonitrile, 1,2-dichloroethane, dimethyl sulfoxide, ethyl acetate, isopropanol, and methyl tert-butyl ether were discovered in the present invention to produce amorphous BisEDT.

In some embodiments, the method further comprises recovering the precipitate to remove impurities. The amorphous product may be separated from the solution, e.g. by precipitation, cooling, filtration, concentration, centrifugation, and combinations thereof, optionally followed by washing with a wash solution, preferably a solvent in which amorphous BisEDT has a very low solubility. The amorphous product can be dried to a constant weight, e.g. at +30° C. to +50° C., preferably at reduced pressure, for, e.g. 10 to 48 hours.

In some embodiments, the bismuth salt is Bi(NO₃)₃.

In some embodiments, 1,2-ethanedithiol is at a concentration of from about 1% wt/vol to about 20% wt/vol prior to step (b). For example, 1,2-ethanedithiol is at a concentration of about 1% wt/vol, about 2% wt/vol, about 3% wt/vol, about 4% wt/vol, about 5% wt/vol, about 6% wt/vol, about 7% wt/vol, about 8% wt/vol, about 9% wt/vol, about 10% wt/vol, about 11% wt/vol, about 12% wt/vol, about 13% wt/vol, about 14% wt/vol, about 15% wt/vol, about 16% wt/vol, about 17% wt/vol, about 18% wt/vol, about 19% wt/vol, or about 20% wt/vol.

In some embodiments, the acidic aqueous solution is prepared by mixing an aqueous suspension of either Bi (III) sub-nitrate or Bi (III) nitrate pentahydrate with an acid under conditions and for a time sufficient to form a substantially clear solution.

In some embodiments, the concentration of either Bi (III) sub-nitrate or Bi (III) nitrate pentahydrate in the aqueous solution is from about 100 mg/mL to about 400 mg/mL. For example, the concentration of either Bi (III) sub-nitrate or Bi (III) nitrate pentahydrate in the aqueous solution is from about 100 mg/mL, about 125 mg/mL, about 150 mg/mL, about 175 mg/mL, about 200 mg/mL, about 225 mg/mL, about 250 mg/mL, about 275 mg/mL, about 300 mg/mL, about 325 mg/mL, about 350 mg/mL, about 375 mg/mL, or about 400 mg/mL.

In some embodiments, the acid is 70% HNO₃. In some embodiments, the acid is 70% HNO₃.

In some embodiments, the method further comprises adding the clear solution to an acidic solution. In some embodiments, the method further comprises adding the clear solution to an acidic solution, wherein the acidic solution is an HNO₃ solution. In some embodiments, the method further comprises adding the clear solution to an acidic solution, wherein the acidic solution is about a 5% HNO₃ solution.

In some embodiments, step (b) is performed at a temperature ranging from about 20° C. to about 28° C. For example, step (b) is performed at a temperature ranging from about 20° C., about 21° C., about 22° C., about 23° C., about 24° C., about 25° C., about 26° C., about 27° C., to about 28° C.

### Pharmaceutical Compositions

In some embodiments, the present disclosure provides a composition comprising an amorphous from of BisEDT.

The compositions and methods of the present disclosure can be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water, physiologically buffered saline, physiologically buffered phosphate, or other solvents or vehicles such as glycols, glycerol, oils such as olive oil, or injectable organic esters. In some embodiments, when such pharmaceutical compositions are for human administration, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as lyophile for reconstitution, powder, solution, syrup, injection or the like. The composition can also be present in a solution suitable for topical administration.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose, or dextrans; antioxidants, such as ascorbic acid or glutathione; chelating agents; low molecular weight proteins; salts; or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols and sugar alcohols, such as glycerin, sorbitol, mannitol, xylitol, erythritol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances, including salts such as sodium chloride, employed in pharmaceutical formulations.

The formulations can conveniently be presented in unit dosage form and can be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect.

In some embodiments, the amorphous BisEDT composition is a powder, spray, ointment, paste, cream, lotion, solution, patch, suspension or gel. In some embodiments, the amorphous BisEDT composition is a suspension.

Ina specific embodiment, the amorphous BisEDT composition is a systemic composition. Indeed, amorphous forms tend to have faster dissolution properties than crystalline forms. In a specific embodiment, the systemic composition may include one or more pharmaceutical carriers or solvents to assist in dissolution of the compound.

In some embodiments, the amorphous BisEDT composition is a solution. In some embodiments, the amorphous BisEDT composition is administered as a dosage from about 0.25 mg/mL to about 15 mg/mL, from about 0.4 mg/mL to about 15 mg/mL, from about 0.6 mg/mL to about 15 mg/mL, from about 0.6 mg/mL to about 100 mg/mL, from about 5 mg/mL to about 100 mg/mL, from about 10 mg/mL to about 100 mg/mL, from about 25 mg/mL to about 100 mg/mL, from about 50 mg/mL to about 100 mg/mL, from about 0.8 mg/mL to about 15 mg/mL, from about 1 mg/mL to about 10 mg/mL, from 2.5 mg/mL to about 10 mg/mL, from about 4 mg/mL to about 10 mg/mL, from about 5 mg/mL to about 10 mg/mL, from about 6 mg/mL to about 10 mg/mL, 0.6 mg/mL to about 6 mg/mL, from about 4 mg/mL to about 15 mg/mL, from about 6 mg/mL to about 15 mg/mL, from about 50 μg/mL to about 750 μg/mL, from about 75 μg/mL to about 500 μg/mL, from about 100 μg/mL to about 250 μg/mL, from about 100 μg/mL to about 150 μg/mL, or from about 75 μg/mL to about 150 μg/mL; and/or the total amount of the BT composition administered to the lungs is from about 0.25 mg to about 15 mg, from about 0.4 mg to about 15 mg, from about 0.6 mg to about 15 mg, from about 0.8 mg to about 15 mg, from about 1 mg to about 10 mg, from 2.5 mg to about 10 mg, from about 4 mg to about 10 mg, from about 5 mg to about 10 mg, from about 6 mg to about 10 mg, 0.6 mg to about 6 mg, from about 4 mg to about 15 mg, from about 6 mg to about 15 mg, from about 50 μg to about 750 μg, from about 75 μg to about 500 μg, from about 100 μg to about 250 μg, from about 100 μg to about 150 μg, or from about 75 μg to about 150 μg. In certain embodiments, the amorphous BisEDT composition is administered as a dosage from about 0.6 mg/mL to about 6 mg/mL.

In some embodiments, the amorphous BisEDT composition is administered three times per day, two times per day, once daily, every other day, once every three days, once every week, once every other week, once monthly, to once every other month. In certain embodiments, the amorphous BisEDT composition is administered once daily. In certain embodiments, the amorphous BisEDT composition is administered once weekly. In certain embodiments, the amorphous BisEDT composition is administered once every other week. In some embodiments, the amorphous BisEDT composition is administered chronically in a 4 week on/4 week off dosing schedule. In some embodiments, the amorphous BisEDT composition is administered chronically, for example as part of a background therapy. As will be appreciated by a person having ordinary skill in the art, the administration frequency may depend on a number of factors including dose and administration route. For example, if the amorphous BisEDT composition is administered via an aerosol administration, a low dose such as 100-1000 μg/mL may be administered once or twice daily; however, a high dose such as 2.5-10 mg/mL may be administered e.g. once or twice a week.

In some embodiments, the amorphous BisEDT composition further comprises one or more carriers selected from animal and vegetable fats, oils, waxes, paraffins, starch, tragacanth, cellulose derivatives, polyethylene glycols, silicones, bentonites, silicic acid, polymers, talc, and zinc oxide. In some embodiments, the carrier is methylcellulose. In some embodiments, the carrier is poly(methyl methacrylate). In some embodiments, the carrier is a synthetic polymer. In a specific embodiment, the synthetic polymer may be one or carbomers.

Compositions can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, by ionizing radiation (gamma photons for example), autoclaving, or by incorporating sterilizing agents in the form of sterile solid compositions that can be dissolved in sterile water, or some other sterile injectable medium immediately before use.

Liquid dosage forms useful for topical administration include pharmaceutically acceptable emulsions, lyophiles for reconstitution, microemulsions, solutions, suspensions, gels, syrups and elixirs. In addition to the active ingredient, the liquid dosage forms can contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (such as cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the topical compositions can also include adjuvants such as wetting agents, emulsifying and suspending agents, and preservative agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, carbomers, and mixtures thereof.

Dosage forms for the topical or transdermal administration include powders, sprays, ointments, pastes, creams, lotions, gels, solutions, patches and inhalants. The active compound can be mixed under sterile conditions with a pharmaceutically acceptable carrier, and with any preservatives or buffers that can be required.

The ointments, pastes, creams and gels can contain, in addition to an active compound, one or more excipients or carriers, such as animal and vegetable fats, oils, waxes, paraffins, starch, tragacanth, cellulose derivatives, polyethylene glycols, silicones, bentonites, silicic acid, talc, polymers, salts, and zinc oxide, or mixtures thereof. In some embodiments, the amorphous BisEDT composition is in the form of an aqueous solution. In some embodiments, the excipient comprises a salt selected from sodium chloride or potassium chloride. In some embodiments, the excipient comprises sodium chloride.

In certain embodiments, the amorphous BisEDT composition is a suspension of amorphous BisEDT in Tween (e.g. Tween 80) and/or in a buffer (e.g. sodium phosphate buffer). For example, in some embodiments, the amorphous BisEDT composition is a suspension of amorphous BisEDT in from about 0.1% Tween 80 to about 1.0% Tween 80, including all ranges therebetween. For example, the amorphous BisEDT composition is a suspension of amorphous BisEDT in about 0.1% Tween 80, about 0.2% Tween 80, about 0.3% Tween 80, about 0.4% Tween 80, about 0.5% Tween 80, about 0.6% Tween 80, about 0.7% Tween 80, about 0.8% Tween 80, about 0.9% Tween 80, or about 1% Tween 80. In some embodiments, the amorphous BisEDT composition is a suspension of amorphous BisEDT in about 0.5% Tween 80.

In a specific embodiment, the present invention may be a pharmaceutical composition comprising amorphous BisEDT suspended therein, wherein the composition comprises a plurality of microparticles. In a specific embodiment, the D90 of said microparticles is less than or equal to 4.5 μm, or 4.0 μm, or 3.5 μm, or 3.0 μm, or 2.5 μm, or 2.0 μm, or 1.9 μm, or 1.8 μm, or μm 1.7 μm, or 1.6 μm, or 1.5 μm or any ranges in between. In a specific embodiment, the D90 of said microparticles is less than or equal to 1.9 μm. In another specific embodiment, the D90 of said microparticles is less than or equal to 1.6 μm. In another specific embodiment, the D50 of said microparticles is less than or equal to 2.5 μm, or 2.0 μm, or 1.5 μm, or 1.3 μm, or 1.2 μm, or 1.1 μm, or 1.0 μm, or 0.9 μm, or 0.87 μm, or 0.72 μm or any ranges in between. In another specific embodiment, the D10 of said microparticles is less than or equal to 0.9 μm, or 0.8 μm, or 0.7 μm, or 0.6 μm, or 0.50 μm, or 0.40 μm, or 0.39 μm, or 0.38 μm, or 0.37 μm, or 0.36 μm, or 0.35 μm, or 0.34 μm, or 0.33 μm, or any ranges in between. In a specific embodiment, the pharmaceutical composition comprising amorphous BisEDT suspended therein, wherein the composition comprises a plurality of microparticles, wherein the D90 of said microparticles is less than or equal to about 1.6 μm. In a specific embodiment, the BT composition comprises BisEDT at a concentration greater than about 0.1 mg/mL, about 0.05% to about 1.0% Tween 80®, about 0.05 to 250 mM sodium chloride, and optionally about 2 to 20 mM sodium phosphate at about pH. 7.4. In another specific embodiment, the compositions described above can be administered to a subject for treating, managing and/or lessening the severity of cystic fibrosis (CF) symptoms and infections in said subject, or any specific method of treating, managing and/or lessening the severity of cystic fibrosis (CF) symptoms described herein. In another specific embodiment, the compositions described above can be administered to a subject for treating, managing and/or lessening the severity of symptoms and infections associated with one or more pulmonary diseases or infections in a subject or any specific method of treating, managing and/or lessening the severity of symptoms and infections associated with one or more pulmonary diseases described herein.

A variety of buffers may be used in the context of the present disclosure and will be readily apparent to a person having ordinary skill in the art. For example, in some embodiments, suitable buffers include sodium or potassium citrate, citric acid, sulfuric acid, phosphate buffers such as sodium phosphate, boric acid, sodium bicarbonate and various mixed phosphate buffers including combinations of Na₂HPO₄, NaH₂PO₄ and KH₂PO₄. In some embodiments, sodium phosphate buffer is used. In some embodiments, sodium citrate buffer is used. Without being bound by any particular theory, changes in airway surface liquid pH may contribute to the host defense defect in cystic fibrosis soon after birth. Changes in lung pH may impact the airway surface liquid environment, improve airway defenses, and alter the disease course. Accordingly, the formulation pH may vary from about 5 to about 10. In some embodiments, the formulation pH is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or about 10. In some embodiments, the formulation pH is about 7.4.

In some embodiments, the amorphous BisEDT composition is a suspension of BisEDT in about 0.5% Tween 80 in sodium phosphate buffer at a pH of about 7.4. In some embodiments, the amorphous BisEDT is present in the composition at a concentration ranging from about 100 μg/mL to about 1000 mg/mL including all integers and ranges therebetween. For example, in some embodiments, amorphous BisEDT is present in the composition at a concentration ranging from about 100 μg/mL, 200 μg/mL, 300 μg/mL, 400 μg/mL, 500 μg/mL, 600 μg/mL, 700 μg/mL, 800 μg/mL, 900 μg/mL, 1000 μg/mL, 10 mg/mL, 25 mg/mL, 50 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, 525 mg/mL, 550 mg/mL, 575 mg/mL, 600 mg/mL, 625 mg/mL, 650 mg/mL, 675 mg/mL, 700 mg/mL, 725 mg/mL, 750 mg/mL, 775 mg/mL, 800 mg/mL, 825 mg/mL, 850 mg/mL, 875 mg/mL, 900 mg/mL, 925 mg/mL, 950 mg/mL, 975 mg/mL, to about 1000 mg/mL. In some embodiments, the amorphous BisEDT is present in the composition at a concentration ranging from about 100 μg/mL to about 1000 μg/mL.

In some embodiments, the composition osmolality may need to be further adjusted with an additive such as NaCl or TDAPS to achieve a desired osmolality. For example, in some embodiments, the osmolality of the composition is adjusted with sodium chloride to an osmolality ranging from about 100 mOsmol/kg to about 500 mOsmol/kg, including all integers and ranges there between. In some embodiments, the osmolality of the composition is from about 290 mOsmol/kg to about 310 mOsmol/kg. For example, in some embodiments, the osmolality of the composition is about 290 mOsmol/kg, 291 mOsmol/kg, 292 mOsmol/kg, 293 mOsmol/kg, 294 mOsmol/kg, 295 mOsmol/kg, 296 mOsmol/kg, 297 mOsmol/kg, 298 mOsmol/kg, 299 mOsmol/kg, 300 mOsmol/kg, 301 mOsmol/kg, 302 mOsmol/kg, 303 mOsmol/kg, 304 mOsmol/kg, 305 mOsmol/kg, 306 mOsmol/kg, 307 mOsmol/kg, 308 mOsmol/kg, 309 mOsmol/kg, to about 310 mOsmol/kg. In some embodiments, the osmolality is about 300 mOsmol/kg.

In some embodiments, the amorphous BisEDT composition is a suspension of BisEDT in Tween (e.g. Tween 80) in a buffer (e.g. sodium phosphate buffer). In some embodiments, the amorphous BisEDT composition is a suspension of BisEDT in about 0.5% Tween 80 in a sodium phosphate buffer at a pH of about 7.4. In some embodiments, the amorphous BisEDT composition is a suspension of BisEDT in about 0.5% Tween 80 in a sodium phosphate buffer at a pH of about 7.4, wherein the composition has an osmolality of about 300 mOsmol/kg (e.g. adjusted to 300 mOsmol/kg with sodium chloride). In some embodiments, the BisEDT is present at a concentration of about 100 μg/mL, 250 μg/mL, 500 μg/mL, 750 μg/mL, 1000 μg/mL, 2.5 mg/mL, 10 mg/mL, 25 mg/mL, 50 mg/mL, 75 mg/mL, or about 100 mg/mL.

In some embodiments, the amorphous BisEDT composition is a suspension formulation which is intended for pulmonary delivery. For example, the composition is a suspension formulation which is ultimately administered by inhalation either orally and/or nasally. Accordingly, in some embodiments, the amorphous BisEDT composition is aerosolized by a device such as a nebulized.

Powders and sprays can contain, in addition to an active compound, excipients such as methylcellulose, sodium chloride, PMMA, lactose, talc, silicic acid, aluminum hydroxide, calcium silicates and polyamide powder, dipalmitoylphosphatidylcholine (DPPC), leucine, polyethyleneglycol, or mixtures of these substances. Sprays can additionally contain customary propellants, such as chlorofluorohydrocarbons and volatile unsubstituted hydrocarbons, such as butane and propane.

In some embodiments, the amorphous BisEDT composition is administered by inhalation, orally or nasally, using an aerosol device, such as a nebulizer. Known nebulizers, such as PARI LC Plus or PARI LC SPRINT, can administer the disclosed compositions as an aqueous solution or suspension, optionally in buffered saline. The solution or suspension can be provided to the subject in the form of a vial or a glass or plastic ampule for use in the nebulizer. The nebulizer can be reusable and includes a compressor that provides the formulation over a period of time, such as about 5-15 minutes or longer. Known compressors, such as APRI Vios Air and DeVilbiss Pulmo-aide, are suitable for administration. The nebulizer administers the formulation topically to the lung tissues, such as mucosa, the bronchi and/or the bronchioles, alveoli, deep lung alveoli. The formulation can penetrate lung mucosa and biofilms to reduce the microbial (e.g. bacterial or fungal) biofilm, impair the growth of the microbial (e.g. bacterial or fungal) biofilm, prevent reformation of the microbial (e.g. bacterial or fungal) biofilm, reduce planktonic growth, and/or inhibit planktonic growth.

In other embodiments, a nose-only aerosol device can be used for administration of the formulation.

An exemplary composition formulation is a neutral pH, isotonic, buffered aqueous solution of amorphous BisEDT microparticles with a nonionic surfactant. In certain embodiments, the buffer is a phosphate buffer with added NaCl. In some embodiments, the microparticle size is a D₅₀ of about 0.1-5 μm. The formulation can be delivered using commercially available compressed air jet nebulizer. In some embodiments, the formulation concentration is about 0.1 μg/mL to about 100 mg/mL.

In some embodiments, the present disclosure provides an aerosol comprising a plurality of dispersed liquid droplets in a gas, said liquid droplets comprising amorphous BisEDT suspended therein, wherein at least 60%, 65%, 70, 75%, 80%, 90%, or 95% of the liquid droplets have a mass median aerodynamic diameter (MMAD) from about 0.03 μm to about 5 μm when measured by laser time of flight and/or cascade impactor. In some embodiments, at least 609%, 65%, 70, 75%, 80%, 90%, or 95% of the liquid droplets have a MMAD of from about 0.03 μm to about 7 μm, or from about 0.5 μm to about 5 μm, or from about 0.7 μm to about 4 μm, or from about 0.7 μm to about 3.5 μm, or from about 0.8 μm to about 3.5 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, including all ranges therebetween. In some embodiments, at least 60%, 65%, 70, 75%, 80%, 90%, or 95% of the liquid droplets have a MMAD of from about 0.8 μm to about 1.6 μm, or from about 0.9 μm to about 3.5 μm, or from about 0.9 μm to about 3 μm, or from about 0.9 μm to about 1.4 μm, or from about 1.0 μm to about 2.0 μm, or from about 1.0 μm to about 1.8 μm, and all ranges therebetween.

In some embodiments, the present disclosure provides an aerosol comprising a plurality of dispersed liquid droplets in a gas, said liquid droplets comprising a BT composition comprising amorphous BisEDT suspended therein; and wherein at least 70% of the liquid droplets have a MMAD from about 0.1 μm to about 3 μm. In some embodiments, prior to aerosolization, the BT composition comprises a plurality of microparticles wherein at least 70%, 80%, or 90% of said microparticles have a VMD from about 0.01 μm to about 2.5 μm. In a specific embodiment, the the microparticles have a VMD less then about 2.5 μm. In some embodiments, the droplets further comprise Tween 80 and optionally a buffer at a pH of about 7.4; and/or sodium chloride.

In some embodiments, the plurality of liquid droplets have a D90 of less than about 10 μm. For example, in some embodiments, the plurality of liquid droplets have a D90 of less than about 10 μm, 9 μm, 8 μm, 7 μm, 6 μm, 5 μm, 4 μm, 3 μm, 2 μm, or about 1 μm. In some embodiments, the plurality of liquid droplets have a D90 of less than about 3 μm. In some embodiments, the plurality of liquid droplets have a D90 ranging from about 0.1 μm to about 5 μm, or about 0.5 μm to about 5 μm, or about 1.0 μm to about 5 μm, or about 2 μm to about 6 μm, or about 2 μm to about 4 μm, or about 2 μm to about 3 μm, or about 1 μm to about 4 μm, or about 1 μm to about 3 μm.

In some embodiments, the plurality of liquid droplets are dispersed in a continuous gas phase.

In some embodiments, amorphous BisEDT is suspended in the liquid droplet. The BT compounds of the present disclosure have little to no solubility in conventional solvents and aerosol carriers and therefore exist substantially as a suspension of amorphous BisEDT particles in the aerosol droplet. For example, in some embodiments, the amorphous BisEDT is less than 1% soluble in the aerosol carrier and therefore exists primarily (>99%) as a solid.

In some embodiments, the droplets further comprise Tween 80 (e.g. from about 0.05% to about 1%) and optionally a buffer (e.g. sodium phosphate or sodium citrate) at a pH of about 7.4; and/or sodium chloride.

The aerosols of the present disclosure have a very narrow MMAD distribution which is beneficial because of the need to concentrate the particle mass in the target size range, and minimize or eliminate the fraction of the product that is outside of the respirable range or ‘fines’, i.e. particles of typically less than 0.4 μm diameter. The ability to create a narrow droplet size distribution in the appropriate size range provides control of the initial evaporation rate and allows for high deposition efficiency. The limiting factor in terms of the lower limit of particle aerosol droplet size is the amorphous BisEDT microparticle size. An aerosolized droplet cannot be smaller than the BisEDT microparticulate size. Furthermore, large microparticle size would not be effective in an aerosolized drug formulation. Large particles would result in large aerosol droplets that when inhaled, would not reach the lunges. Indeed, aerosols with an MMAD above about 5 to 10 microns in diameter will not reach the appreciably, but instead will just reach and impact the the mouth, throat, and upper airways/bronchi.

As such, the microparticle size distribution, as well as the uniformity and consistent reproducibility of the microparticulate size distribution, are important beneficial characteristics to support the generation of a safe, effective, and efficient aerosolized BisEDT drug product for inhalation purposes. Accordingly, in some embodiments, the aerosols of the present disclosure effectuate a deposition efficiency of greater than 3%, greater than 5%, greater than 10%, greater than 15%, greater than 20%, greater than 25%, greater than 30%, greater than 35% m greater than 40%, greater than 45%, greater than 50%, greater than 55%, greater than 60%, greater than 65%, greater than 70%, greater than 75%, and greater than 80%. In some embodiments, the deposition efficiency refers to deposition to the deep lung region of lung, for example, to the deep lung alveoli. In some embodiments, the aerosols of the present disclosure effectuate a deposition efficiency upon aerosolization via a nebulizer. For example, the nebulizer is a jet nebulizer. In some embodiments, the jet nebulizer is a Pari LC Plus jet nebulizer or Pari LC SPRINT jet nebulizer. In some embodiments, the nebulizer has an inlet pressure from about 10 to about 40 psig (e.g. 20-25 psig). In some embodiments, the inlet flow is from about 3 L/min to about 8 L/min (e.g. 5.2 L/min). In some embodiments, the exhaust air flow is from about 3 L/min to about 8 L/min (e.g. 5 L/min).

The alveolar region of the lung has a minimal thickness (0.5 μm-2.5 μm) separating the blood flow from the lumen so conventional pulmonary agents that deposit on the alveolar epithelium have extremely short lung residence time due to systemic absorption. Accordingly, conventional pulmonary treatments typically require frequent dosing in order to maintain adequate levels of drug at the tissue level. However, the aerosolized particles of the present disclosure were surprisingly discovered to possess an exceptionally long residence time in the lungs (measured as half-life) and have reduced mucociliary clearance and macrophage uptake relative to conventional pulmonary treatments. Furthermore, the long residence time of the aerosols of the present minimizes systemic activity and associated systemic side effects. Without being bound by any particular theory, it is believed that the aerosolized microparticles dissolve slowly on the lung lumen and the systemic exposure is thus dissolution rate limited. Further, the increased lung residence time results in significant reductions in microbial colony due to the continuous presence of the BT microparticles.

In another embodiment, after delivering the aerosolized composition to a subject, at least 60%, 65%, 70, 75%, 80%, 90%, or 95% of the dose is deposited on the lung, as opposed to the orpharanygeal region and the conducting airways. In a specific embodiment, at least 80% of the dose is deposited on the lung, as opposed to the orpharanygeal region and the conducting airways. In another specific embodiment, at least 90% of the dose is deposited on the lung, as opposed to the orpharanygeal region and the conducting airways.

In some embodiments, the present disclosure provides a method of treating, managing or lessening the severity of cystic fibrosis (CF) symptoms and infections in a subject, the method comprising administering to the subject a composition that comprises amorphous BisEDT wherein the composition is a suspension of microparticles having a volumetric mean diameter (VMD) from about 0.4 μm to about 5 μm and/or a mass median aerodynamic diameter (MMAD) from about 0.4 μm to about 5 μm. In some embodiments, the composition comprises BisEDT at a concentration greater than about 0.1 mg/mL, about 0.05% to about 1.0% Tween 80®, about 40 mM to about 200 mM sodium chloride, and optionally about 2 to 20 mM sodium phosphate at about pH. 7.4. For example, in some embodiments, the composition comprises BisEDT at a concentration greater than about 0.25 mg/mL, about 0.5% Tween 80®, about 10 mM sodium chloride, and about 10 mM sodium phosphate at about pH 7.4. In another embodiment of the methods herein, the composition is administered by aerosolization

In another embodiment, after delivering the aerosolized composition to a subject, at least 60%, 65%, 70%, 75%, 80%, 90%, or 95% of the dose is deposited on the lung, as opposed to the orpharanygeal region and the conducting airways. In a specific embodiment, at least 80% of the dose is deposited on the lung, as opposed to the orpharanygeal region and the conducting airways. In another specific embodiment, at least 90% of the dose is deposited on the lung, as opposed to the orpharanygeal region and the conducting airways. In a specific embodiment, the subject is a rat. In another specific embodiment, the percent deposition is determined using a Pari LC plus jet nebulizer to administer to the rats with the formulations described herein.

In another embodiment, the methods of the present invention may include treating, managing or lessening the severity of cystic fibrosis (CF) symptoms and infections in a subject, by administering to the subject a composition that comprises amorphous BisEDT, wherein the composition is a suspension of microparticles having a volumetric mean diameter (VMD) from about 0.01 μm to about 5 μm and/or a mass median aerodynamic diameter (MMAD) from about 0.01 μm to about 5 μm.

In some embodiments, the composition is a suspension of amorphous BisEDT microparticles having a volumetric mean diameter (VMD) from about 0.01 μm to about 5 μm. In some embodiments, at least 60%, 65%, 70%, 75%, 80%, 90%, or 95% of the microparticles have a VMD of less than about 5 μm, less than about 4 μm, less than about 3 μm, less than about 2 μm, less than about 1 μm, from about 0.01 μm to about 5 μm, from about 0.1 μm to about 5 sm from about 0.4 μm to about 5 μm, or from about 0.6 μm to about 2.5 μm, or from about 0.7 μm to about 4 μm, or from about 0.7 μm to about 3.5 μm, or from about 0.7 μm to about 3.0 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm and all ranges therebetween. In some embodiments, at least 60%, 65%, 70%, 75%, 80%, 90%, or 95% of the microparticles have a VMD of less than about 5 μm, less than about 4 μm, less than about 3 μm, less than about 2 μm, less than about 1 μm, from about 0.01 μm to about 5 μm, from about 0.1 μm to about 5 μm, from about 0.6 μm to about 2.5 μm, or from about 0.8 μm to about 1.6 μm, or from about 0.9 μm to about 3.5 μm, or from about 0.9 μm to about 3 μm, or from about 0.9 μm to about 1.4 μm, or from about 1.0 μm to about 2.0 μm, or from about 1.0 μm to about 1.8 μm and all ranges therebetween. In some embodiments, the microparticles have a D90 of less than about 10 μm. For example, in some embodiments, the microparticles have a D90 of less than about 10 μm, 9 μm, 8 μm, 7 μm, 6 μm, 5 μm, 4 μm, 3 μm, 2 μm, or about 1 μm. In some embodiments, the microparticles have a D90 of less than about 3 μm. In some embodiments, the microparticles have a D90 ranging from about 0.01 μm to about 5 μm, or about 0.1 μm to about 5 μm, or about 1 μm to about 5 μm, or about 2 μm to about 6 μm, or about 2 μm to about 4 μm, or about 2 μm to about 3 μm, or about 1 μm to about 4 μm, or about 1 μm to about 3 μm.

In some embodiments, the composition is aerosolized, wherein the aerosolized liquid droplets have a MMAD of less than about 5 μm, less than about 4 μm, less than about 3 μm, less than about 2 μm, less than about 1 μm, from about 0.01 μm to about 5 μm, from about 0.1 μm to about 5 μm, from about 0.4 μm to about 5 μm. In some embodiments, at least 60%, 65%, 70%, 75%, 80%, 90%, or 95% of the liquid droplets have a MMAD of less than about 5 μm, less than about 4 μm, less than about 3 μm, less than about 2 μm, less than about 1 μm, from about 0.01 μm to about 5 μm, from about 0.1 μm to about 5 μm, of from about 0.4 μm to about 7 μm, or from about 0.5 μm to about 5 μm, or from about 0.7 μm to about 4 μm, or from about 0.7 μm to about 3.5 μm, or from about 0.8 μm to about 3.5 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm and all ranges therebetween. In some embodiments, at least 60%, 65%, 70%, 75%, 80%, 90%, or 95% of the liquid droplets have a MMAD of from about 0.8 μm to about 1.6 μm, or from about 0.9 μm to about 3.5 μm, or from about 0.9 μm to about 3 sm, or from about 0.9 μm to about 1.4 μm, or from about 1.0 μm to about 2.0 μm, or from about 1.0 μm to about 1.8 μm and all ranges therebetween. In some embodiments, the plurality of liquid droplets have a D90 of less than about 10 μm. For example, in some embodiments, the plurality of liquid droplets have a D90 of less than about 10 μm, 9 μm, 8 μm, 7 μm, 6 μm, 5 μm, 4 μm, 3 μm, 2 μm, or about 1 μm. In some embodiments, the plurality of liquid droplets have a D90 of less than about 3 μm. In some embodiments, the plurality of liquid droplets have a D90 ranging from about 0.01 μm to about 5 μm, or about 0.1 μm to about 5 μm, or about 1 μm to about 5 μm, or about 2 μm to about 6 μm, or about 2 μm to about 4 μm, or about 2 μm to about 3 μm, or about 1 μm to about 4 μm, or about 1 μm to about 3 μm.

In some embodiments, the plurality of liquid droplets are dispersed in a continuous gas phase.

In some embodiments of the presently disclosed compositions, at least 60%, 65%, 70%, 75%, 80%, 90%, or 95% of the microparticles have a volumetric mean diameter of of less than about 5 μm, less than about 4 μm, less than about 3 μm, less than about 2 μm, less than about 1 μm, from about 0.01 μm to about 5 μm, from about 0.1 μm to about 5 μm or about 0.6 μm to about 2.5 μm. In some embodiments, substantially all of the microparticles have a VMD of of less than about 5 μm, less than about 4 μm, less than about 3 μm, less than about 2 μm, less than about 1 μm, from about 0.01 μm to about 5 μm, from about 0.1 μm to about 5 μm, or from about 0.6 μm to about 2.5 μm. In some embodiments, at least 70% of the aerosolized particles have a MMAD of less than about 5 μm, less than about 4 μm, less than about 3 μm, less than about 2 μm, less than about 1 μm, from about 0.01 μm to about 5 μm, from about 0.1 μm to about 5 μm, or of about 0.9 μm to about 3 μm. In some embodiments, the composition is a suspension of microparticles having a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, from about 0.6 μm to about 2.5 μm and/or a mass median aerodynamic diameter (MMAD) of less than about 5 μm, less than about 4 μm, less than about 3 μm, less than about 2 μm, less than about 1 μm, from about 0.01 μm to about 5 μm, from about 0.1 μm to about 5 μm. or from about 0.9 μm to about 3 μm. In some embodiments, the composition comprises a plurality of microparticles that comprise a amorphous BisEDT, substantially all of said microparticles having a volumetric mean diameter of from about 0.1 μm to about 5 μm.

In some embodiments, the composition is aerosolized via a nebulizer. For example, the nebulizer is a jet nebulizer or vibrating mesh nebulizer. In some embodiments, the jet nebulizer is a Pari LC Plus jet nebulizer or Pari LC SPRINT jet nebulizer. In some embodiments, the nebulizer has an inlet pressure from about 10 to about 40 psig (e.g. 20-25 psig). In some embodiments, the inlet flow is from about 3 L/min to about 8 L/min (e.g. 5.2 U/min). In some embodiments, the exhaust air flow is from about 3 L/min to about 8 L/min (e.g. 5 L/min).

In some embodiments, the present disclosure provides a pharmaceutical composition comprising bismuth-thiol (BT) composition that comprises amorphous BisEDT suspended therein, wherein the BT composition comprises a plurality of microparticles, wherein the D90 of said microparticles is less than or equal to 1.9 μm. In some embodiments, the BT composition comprises a plurality of microparticles, wherein the D90 of said microparticles is less than or equal to about 1.6 μm. In some embodiments, at least 70%, 80%, or 90% of said microparticles have a volumetric mean diameter from about 0.6 μm to about 2.5 μm.

In some embodiments, the present disclosure provides a pharmaceutical composition comprising bismuth-thiol (BT) composition that comprises amorphous BisEDT suspended therein, wherein the BT composition comprises a plurality of microparticles, wherein the D90 of said microparticles is less than or equal to 1.9 μm. In some embodiments, the BT composition comprises a plurality of microparticles, wherein the D90 of said microparticles is less than or equal to about 1.6 μm. In some embodiments, the present disclosure provides a method for healing a wound in a subject having a diabetic foot infection, comprising administering the subject a therapeutically effective amount of the composition.

Examples of suitable aqueous and nonaqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents, chelating agents, and dispersing agents can also be desirable to include isotonic agents, such as sugars, sodium chloride, and the like into the compositions. In addition, prolonged absorption of the injectable pharmaceutical form can be brought about by the inclusion of agents that delay absorption such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution, which, in turn, can depend upon crystal size and crystalline form. Alternatively, delayed absorption of a parenterally administered drug form is accomplished by dissolving or suspending the drug in an oil vehicle.

Actual dosage levels of the active ingredients in the pharmaceutical compositions can be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount can include, but are not limited to, the severity of the subject's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable dose of an active compound used in the compositions and methods of the disclosure will be that amount of the compound that is the lowest dose effective to produce a therapeutic effect. Such an effective dose will generally depend upon the factors described above.

This disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives, chelating agents, and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Combination Treatments

In certain embodiments, amorphous BisEDT can be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of amorphous BisEDT with one or more additional therapeutic agent(s) provides improved efficacy relative to each individual administration of the compound of the disclosure or the one or more additional therapeutic agent(s). In certain such embodiments, the conjoint administration provides an additive effect or synergistic effect, wherein an additive effect refers to the sum of each of the effects of individual administration of the compound of the disclosure and the one or more additional therapeutic agent(s). In some embodiments, the subject receives conjoint administration of a therapy for another disease, disorder, or condition. In some embodiments, the other therapy is a CFTR modulator or bronchodilator.

In some embodiments, the methods of the present disclosure comprise coadministering or conjointly administering to the subject an antibiotic selected from amikacin, tobramycin, gentamicin, piperacillin, mezlocillin, ticarcillin, imipenum, ciprofloxacin, ceftazidime, aztreonam, ticaricillin-clavulanate, dicloxacillin, amoxicillin, ticarcillin-clavulanate, trimethoprim-sulfamethoxazole, cephalexin, piperacillin-tazobactam, linezolid, daptomycin, vancomycin, metronidazole, clindamycin, colistin, tetracycline, levofloxacin, amoxicillin and clavulanic acid (Augmentin®), cloxacillin, dicloxacillin, cefdinir, cefprozil, cefaclor, cefuroxime, erythromycin/sulfisoxazole, erythromycin, clarithromycin, azithromycin, doxycycline, minocycline, tigecycline, imipenem, meripenem, colistimethate/colistin®, methicillin, oxacillin, nafcillin, cabenicillin, azlocillin, piperacillin and tazobactam (Zosyn®), cefepime, ethambutol, rifampin, and meropenem. In some embodiments, the antibiotic is selected from meropenem, ceftazidime, tobramycin, amikacin, aztreonam, ciprofloxacin, colistin, and levofloxacin.

In certain embodiments of the present disclosure, the therapeutic agents that can be conjointly administered with camorphous BisEDT, include known antibiotics. In some embodiments, the antibiotic is selected from methicillin, vancomycin, nafcillin, gentamicin, ampicillin, chloramphenicol, doxycycline, colistin amikacin, aztreonam, and tobramycin. In some embodiments, the antibiotic is selected from tobramycin, imipenem, tetracycline, and minocycline. In some embodiments, the antibiotic is administered systemically after revision surgery. In some embodiments, the antibiotic is administered prior to revision surgery. The conjointly administered therapeutic agent, such as an antibiotic, can be administered with any suitable frequency and at any suitable dosage. Such dosage amount and frequency can be determined by those of ordinary skill in the art.

In certain embodiments, amorphous BisEDT can be conjointly administered with one or more other BT compounds. Moreover, such combinations can be conjointly administered with other therapeutic agents.

### Methods of Treating Pulmonary Infections

Cystic fibrosis (CF), an autosomal recessive disorder, is caused by functional deficiency of the cAMP-activated plasma membrane chloride channel, cystic fibrosis transmembrane conductance regulator (CFTR), which results in pulmonary and other complications.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation.

In healthy individuals, clearance of lung bacteria relies on the concerted action of two anatomic features: (i) the ciliated apical surface of the airway epithelium and (ii) a mucus layer that lines the airway lumen. The airway cilia beat synchronously, creating a steady current that continually moves the mucus layer upward toward the nasopharynx. The mucus layer is biphasic, consisting of an upper, viscous layer that serves to trap particulates and microorganisms and a lower, more fluid layer in which the cilia beat. When functioning normally, this clearance system traps foreign bodies in the mucus and subsequently carries them to the nasopharynx, where they are expectorated and swallowed.

However, abnormal secretory characteristics of the CF airway cells due to the ion imbalance caused by the mutant CFTR protein alter the viscosity of the airway fluid, such that the normally serous “periciliary” layer becomes thicker, inhibiting escalator action that clears foreign bodies. Bacteria are trapped in the mucous and result in an ongoing infection in the lungs.

CF patients routinely produce sputum from the lungs through coughing, aided by other physical therapies designed to free mucous from the lungs. Many of the organisms that are isolated from CF sputum are pathogens that often benignly colonize the upper respiratory tract (e.g., non-typeable H. influenzae) or the nose (e.g., S. aureus) or are common environmental organisms that behave as pathogens only under certain opportunistic situations (e.g., P. aeruiginosa). Different bacteria and the level of infection in the lungs can be determinative of a CF patient's symptoms and outcome. For example, the presence of S. aureus and the absence of P. aeruginosa predicts long term survival in CF patients after the age of 18 years. In addition, the potential for increasing P. aeruginosa colonization as a consequence of suppression of S. aureus infection may be relevant for some patients.

Of all the bacteria that can colonize in the lungs of CF patients, chronic P. aeruginosa airway infection and the accompanying inflammatory response are the major clinical problems for CF patients today. While antibiotic chemotherapy and chemoprophylaxis have reduced the morbidity and early mortality of CF patients from this infection, the intrinsic ability of P. aeruginosa to develop resistance to many commonly used antibiotics probably contributes to the inability to eradicate P. aeruginosa from CF patients' lung and ultimately allows this microbe to be highly problematic for these patients.

CF patients can acquire P. aeruginosa in their respiratory tracts at any time, with most studies indicating that 70 to 80%/6 CF patients are infected by their teen years. P. aeruginosa infection probably initially occurs within the first 3 years of life. After the onset of chronic infection, patients experience episodic exacerbations that can benefit from antibiotic chemotherapy. Infection may result from social contacts or may be hospital acquired, but the diversity of P. aeruginosa clones isolated from CF patients suggests that most clinical isolates originate in the environment. CF patients chronically infected by P. aeruginosa show a steeper lung function decline (expressed as forced expired volume in 1 second (FEVI) decline over time), a higher number of pulmonary exacerbations, more hospital admissions and higher mortality than P. aeruginosa-free patients. The effects of P. aeruginosa are more severe if chronic infection develops early.

P. aeruginosa infections can change over time to develop a mucoid phenotype, which can initiate the chronic-infection stage of cystic fibrosis. The mucoid phenotype results from bacterial production of a polysaccharide known as both alginate and mucoid exopolysaccharide (MEP) and plays an important role in bacterial evasion of the host immune response. The MEP/alginate itself is able to promote bacterial survival in the face of host immune effectors. Alginate overproduction by P. aeruginosa correlates with the onset of significant deterioration in lung function. In addition, P. aeruginosa can grow as a biofilm, which increases bacterial resistance to phagocytic action and antibiotic efficacy.

Bacterial biofilms are a matrix of cells that adhere to each other and often a surface, such as lung mucosa. The bacterial cells become embedded within an extracellular matrix formed from extracellular polymeric substances, such as polysaccharides, proteins, lipids and DNA. Biofilm bacterial cells are physiologically different than planktonic cells in which a large number of genes are differentially regulated. Biofilms can also be more resistant to antibiotics given the shelter provided by the matrix. Biofilms of P. aeruginosa and other bacteria that are present in the lungs of CF patients increase the difficulty of successful infection management and reduction. Combinations of CF-relevant bacteria forming multispecies biofilms containing P. aeruginosa have demonstrated greater resistance, virulence and pathogenicity than comparable single-species biofilms. The presence of such complex biofilms in the lungs of CF patients is considered to be largely responsible for the chronic, persistent nature of these pulmonary infections, which are not only responsible for chronic, ongoing and progressive morbidity, but are also ultimately responsible for mortality in this population.

In addition to P. aeruginosa, other pathogens commonly found in CF patients' lungs include, but are not limited to, Haemophilus influenzae, Staphylococcus aureus, Staphylococcus warneri, Staphylococcus lugdunensis, Staphylococcus epidermidis, Streptococcus milleri/anginous, Streptococcus pyogenes, non-tuberculosis mycobacterium, Mycobacterium tuberculosis, Burkholderia spp., Achromobacter xylosoxidans, Pandoraea sputorum, Stenotrophomonas maltophilia, Alcaligenes xylosoxidans, Haemophilus pittmaniae, Serratia marcescens, Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, Aspergillus flavus, Morganella morganii, Inquilinus limosus, Ralstonia mannitolilytica, Pandoraea apista, Pandoraea pnomenusa, Pandoraea sputorum, Bdellovibrio bacteriovorus, Bordetella bronchiseptica, Vampirovibrio chlorellavorus, Actinobacter baumanni, Cupriadidus metallidurans, Cupriavidus pauculus, Cupriavidus respiraculi, Delftia acidivordans, Exophilia dermatitidis, Herbaspirillum frisingense, Herbaspirillum seropedicae, Klebsiella pneumoniae, Pandoraea norimbergensis, Pandoraea pulmonicola, Pseudomonas mendocina, Pseudomonas pseudoalcaligenes, Pseudomonas putida, Pseudomonas stutzeri, Ralstonia insidiosa, Ralstonia pickettii, Neisseria gonorrhoeae, NDM-1 positive E. coli, Enterobacter cloaca, Vancomycin-resistant E. faecium, Vancomycin-resistant E. faecalis, E. faecium, E. faecalis, Clindamycin-resistant S. agalactiae, S. agalactiae, Bacteroides fragilis, Clostridium difficile, Streptococcus pneumonia, Moraxella catarrhalis, Haemophilus haemolyticus, Haemophilus parainfluenzae, Chlamydophilia pneumoniae, Mycoplasma pneumoniae, Atopobium spp., Sphingomonas spp., Saccharibacteria spp., Leptotrichia spp., Capnocytophaga, Oribacterium spp., Aquabacterium spp., Lachnoanaerobaculum spp., Campylobacter spp., Acinetobacter spp., Agrobacterium spp., Bordetella spp., Brevundimonas spp., Chryseobacterium spp., Delftia spp., Enterobacter spp., Klebsiella spp., Pandoraea spp., Pseudomonas spp., Ralstonia spp., and Prevotella spp.

Exemplary non-tuberculosis mycobacterium include, but are not limited to, Mycobacterium abscessus, Mycobacterium avium, Mycobacterium intracellulare, Mycobacterium fortuitum, Mycobacterium gordonae, Mycobacterium kansasii, Mycobacterium avium complex (MAC), Mycobacterium abscessus complex (MABSC) Mycobacterium marinum, Mycobacterium terrae and Mycobacterium cheloni.

Exemplary species of Burkholderia include, but are not limited to, Burkholderia cepacia, Burkholderia multivorans, Burkholderia cenocepacia, Burkholderia stabilis, Burkholderia vietnamiensis, Burkholderia dolosa, Burkholderia ambifaria, Burkholderia anthina, Burkholderia pyrrocinia, Burkholderia gladioli, Burkholderia ubonensis, Burkholderia arboris, Burkholderia latens, Burkholderia lata, Burkholderia metallica, Burkholderia seminalis, Burkholderia contaminans, and Burkholderia diffusa.

In some embodiments, the bacterial pathogen is selected from Pseudomonas aeruginosa, multi drug-resistant Pseudomonas aeruginosa, Staphylococcus aureus, multi drug-resistant Staphylococcus aureus, methicillin resistant Staphylococcus aureus, Mycobacterium abscessus, Mycobacterium avium, Burkholderia cepacia, Burkholderia multivorans, Burkholderia cenocepacia, Burkholderia dolosa, Achromobacter xylosoxidans, Stenotrophomonas maltophilia Staphylococcus epidermidis, and Burkholderia vietnamiensis. In certain embodiments, the bacterial pathogen is selected from Haemophilus influenzae, Pseudomonas aeruginosa, and Staphylococcus aureus. In certain embodiments, the bacterial pathogen is selected from biofilms of Pseudomonas aeruginosa, Burkholderia cenocepacia, Burkholderia cepacia complex, Mycobacterium abscessus, Mycobacterium avium, Achromobacter spp., Staphylococcus epidermidis, Stenotrophomonas maltophilia, and Staphylococcus aureus.

In some embodiments, the bacterial pathogen exhibits resistance to one or more antibiotics. Methicillin-resistant S. aureus (MRSA) is an example of a singly resistant strain that is difficult to treat in CF patients and the population at large, while even more challenging multi-drug resistant (MDR) strains can occur in bacteria such as P. aeruginosa and S. aureus. For example, a bacterial pathogen can become resistant to known standards of antibiotic care, including, but not limited to, amikacin, aztreonam, methicillin, vancomycin, nafcillin, gentamicin, ampicillin, chloramphenicol, doxycycline and tobramycin. In some embodiments, the resistant antibiotic is amikacin, aztreonam, or tobramycin.

Long-term, repeated treatment with antibiotics to treat CF-associated infections typically results in development of antibiotic-resistance, characterized by the presence of microbial biofilms. Recent research has repeatedly demonstrated a correlation between multi-drug resistant (MDR) bacteria, and stronger, more prolific biofilm-forming capabilities. Biofilm involvement in the lung is considered highly immunogenic, accelerating structural lung damage. Further, bacteria within biofilms are protected from antibiotics, which increases the minimal inhibitory concentration of such antibiotics. Biofilms tend to reduce the antimicrobial activity of aminoglycosides and beta-lactam antibiotics by both changing the pH of the respiratory mucosa and through the production of beta-lactamase enzymes. The involvement of biofilm-forming bacteria in CF is correlated with decreased lung function and reduced Quality of Life, decreased response to antibiotic therapy, increased exacerbations, and, over time, reduced survival.

In some embodiments, the BT composition is administered by inhalation, either orally or nasally, using an aerosol device, such as a nebulizer. A nebulizer can administer the BT composition topically to the lung tissue, which can include the lung mucosa, the alveoli (e.g. deep lung alveoli), the bronchi and/or the bronchioles. Thus, in some embodiments, the present disclosure provides for administration of the amorphous BisEDT composition to the deep lung region of the lung (e.g. the deep lung alveoli). Local topical administration of the BT composition provides several key advantages over systemic antibiotic therapies. The term “systemic” refers to administration of a medication into the circulatory system of the subject such that the majority of the entire body can be exposed. Systemic administration of a medication can occur enterally (absorption through the gastrointestinal tract, e.g. oral administration) or parenterally (absorption through injection or infusion, e.g. intravenously).

Systemic anti-infective products have several disadvantages relating to the treatment of localized infections, including: (a) difficulty in achieving a therapeutically effective concentration at the site of local infection, particularly in the case of infections associated with topical locations, such as pulmonary airways, (b) frequent unintended toxic effects on organ systems exposed through systemic circulation, and (c) increased generation of antibiotic-resistant bacteria as a result of the widespread exposure of the body's normal flora to the anti-infective agent, (d) resulting in greater likelihood of transmission of such antibiotic-resistant bacteria to others as a result of long term sustained/repeated exposure of the entire body's complement of normal flora and (e) reduced preservation of the beneficial influence of the healthy normal flora throughout the body due to extensive systemic exposure.

Oral dosing can result in high plasma concentrations that may lead to toxicity and varied inter-patient exposure (variable absorption and variable first-pass, hepatic clearance) or drug-drug interactions. When orally dosing to treat a lung infection, high plasma exposure is necessary to achieve therapeutic exposure in the lungs. In contrast, inhaled dosing for topical lung indications requires a lower total dose to achieve an efficacious concentration because the drug is administered directly (topically) to the site of infection, which results in significantly less systemic exposure. Inhaled dosing achieves higher local concentrations in the lung that significantly exceed the MIC of the drug over a long period of time. Due to the delivery of high concentrations of drug directly to the lung, the achieved pulmonary concentrations following inhalation may greatly exceed those achieved by oral dosing. From previous experience with inhaled antibiotics, the lung concentration of drug upon inhalation is >100× greater than upon systemic/oral administration of the same dose.

In some embodiments, one or more of the following symptoms (e.g. cystic fibrosis-related symptoms) is lessened in severity in the subject: cough, wheezing, breathlessness, bronchiectasis, nasal polyps, hemoptysis, respiratory failure, and pulmonary exacerbation. Additional non-cystic fibrosis related infections may also be lessened in severity. Thus, inhaled formulations of the BT compositions disclosed herein provide a more targeted and effective antibiotic treatment than a corresponding formulation administered systemically.

BisEDT is known broad-spectrum antimicrobial (and anti-biofilm) small molecule drug product for the treatment of chronic, ultimately life-threatening pulmonary infections secondary to CF. Its efficacy extends to Gram-positive, antibiotic-resistant pathogens including methicillin-resistant Staphylococcus aureus (MRSA, including community-associated [CA]-MRSA), methicillin-resistant Staphylococcus epidermidis (MRSE), and vancomycin-resistant Enterococcus (VRE). BT compounds are also potent against Multi-drug-resistant (MDR) Gram-negative pathogens including Pseudomonas aeruginosa, Escherichia coli, Klebsiella pneumoniae (including, in all of the afore-mentioned bacteria, carbapenem-resistant strains), and Acinetobacter baumannii.

BisEDT have the dual ability to overcome a) a very diversified spectrum of antibiotic resistance profiles (due to evolution/diversification driven by persistence, time and isolation in many different anatomical regions throughout the pulmonary airways), and b) antibiotic-resistant and MDR biofilms.

Disclosed herein are methods of treating, managing or lessening the severity of cystic fibrosis (CF) symptoms and infections in a subject, the method comprising administering to the subject a bismuth-thiol (BT) composition that comprises amorphous BisEDT. Also disclosed herein are methods of treating, managing or lessening the severity of symptoms and infections associated with one or more pulmonary diseases or infections in a subject, including non-CF associated diseases, the method comprising administering to the subject a bismuth-thiol (BT) composition that comprises amorphous BisEDT. In some embodiments, the subject has at least one pulmonary infection, such as a CF-related pulmonary infection. In other embodiments, the subject has at least two pulmonary infections and the infections are either concurrent or successive in order. The pulmonary infections could be cause by the same microbial pathogen and be located in two different lungs, or lobes of the lung. In other embodiments, the pulmonary infections could be caused by different microbial pathogens and be located in the same lung, or lobe of the lung. In some embodiments, the pulmonary infection is in one lung, while in others it is present in both lungs. In certain embodiments, the pulmonary infection is in one or more of the three lobes of the right lung. In other embodiments, the pulmonary infection is in one or both of the two lobes of the left lung. Any combination of one or more microbial pathogens, microbial pathogen quantity, and infection location in the lung is contemplated within the term “pulmonary infection”. In some embodiments, the pulmonary infection is a bronchiectasis infection, pneumonia, valley fever, allergic bronchopulmonary aspergillosis (ABPA), ventilator acquired pneumonia, hospital acquired pneumonia, community acquired pneumonia, ventilator associated tracheobronchitis, lower respiratory tract infection, non-tuberculous Mycobacteria, anthrax, legionellosis, pertussis, bronchitis, Bronchiolitis, COPD-associated infection, and post-lung transplantation. In some embodiments, the pulmonary infection is a bronchiectasis infection.

In some embodiments, the pulmonary infection contains one or more bacterial or fungal pathogens. In some embodiments, the disclosed methods comprise treating the CF-related pulmonary infection. In some embodiments, the disclosed methods comprise managing the CF-related pulmonary infection. In some embodiments, the disclosed methods comprise lessening the severity of the CF-related pulmonary infection.

In some embodiments, the methods of the present invention may include treating, managing or lessening the severity of symptoms and infections associated with one or more pulmonary diseases or infections in a subject by administering to the subject a bismuth-thiol (BT) composition that comprises amorphous BisEDT

In certain embodiments, the pulmonary infection is located in or on the lung mucosa, the bronchi and/or the bronchioles. In other embodiments, the pulmonary infection is located on the surface of or within a bacterial biofilm, aggregated bacteria, a fungal biofilm, or aggregated fungi. In some embodiments, the pulmonary infection is located in the sputum wherein the pulmonary infection involves and is, at least in part, present in the mucous/sputum layers associated with the lungs. In certain embodiments, the bacterial pathogen comprises one or more of gram-positive bacteria and gram-negative bacteria. The bacterial pathogen can comprise one or more of a bacterial biofilm and planktonic bacteria. In some embodiments, the fungal pathogen comprises one or more of a fungal biofilm and planktonic fungi. In certain embodiments, the fungal pathogen is Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, and/or Aspergillus flavus.

In some embodiments, the method comprises at least one of: (i) reducing the microbial (e.g. bacterial or fungal) biofilm, (ii) impairing growth of the microbial (e.g. bacterial or fungal) biofilm, and (iii) preventing reformation of the microbial (e.g. bacterial or fungal) biofilm. In other embodiments, the amorphous BisEDT manages or lessens the severity of the pulmonary infection by one or more of.
prevention of the infection by the bacterial or fungal pathogen;
prevention of elaboration or secretion of exotoxins from the bacterial or fungal pathogen;
reduction of the bacterial or fungal pathogen (e.g. as measure by amount or titer);
inhibition of cell viability or cell growth of planktonic cells (e.g. substantially all of the cells) of the bacterial or fungal pathogen;
inhibition of biofilm formation by the bacterial or fungal pathogen;
inhibition of biofilm viability or biofilm growth of biofilm-form cells (e.g. substantially all of the cells) of the bacterial or fungal pathogen; and
reducing the viscosity of the sputum.

In some embodiments, the bismuth-thiol composition comprises a plurality of microparticles that comprise amorphous BisEDT, substantially all of said microparticles having a volumetric mean diameter of less than about 5 μm, less than about 4 μm, less than about 3 μm, less than about 2 μm, less than about 1 μm, from about 0.01 μm to about 5 μm, from about 0.1 sm to about 5 μm, from about 0.4 μm to about 5 μm, and wherein the BT compound comprises amorphous BisEDT. In some embodiments, at least 60%/6, 65%, 70, 75%, 80%, 90%, or 95% of the microparticles have a volumetric mean diameter of less than about 5 μm, less than about 4 μm, less than about 3 μm, less than about 2 μm, less than about 1 μm, from about 0.01 μm to about 5 μm, from about 0.1 μm to about 5 μm, or of from about 0.4 μm to about 3 μm, or from about 0.5 μm to about 2 μm, or from about 0.7 μm to about 2 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, or any narrow ranges between the specific ranges described above.

In some embodiments of the presently disclosed methods, at least 60%, 65%, 70, 75%, 80%, 90%, or 95% of the microparticles have a volumetric mean diameter of less than about 5 μm, less than about 4 μm, less than about 3 μm, less than about 2 μm, less than about 1 μm, from about 0.01 μm to about 5 μm, from about 0.1 μm to about 5 μm or of from about 0.01 μm to about 2.5 μm. In some embodiments, substantially all of the microparticles have a VMD of from about 0.1 μm to about 2.5 μm or less than about 5 μm, less than about 4 μm, less than about 3 μm, less than about 2 μm, less than about 1 μm, from about 0.01 μm to about 5 μm, or from about 0.1 μm to about 5 μm. In some embodiments, at least 70% of the aerosolized particles have a MMAD of about 0.01 μm to about 3 μm. In some embodiments, the composition is a suspension of microparticles having a volumetric mean diameter (VMD) from about 0.1 μm to about 2.5 μm and/or a mass median aerodynamic diameter (MMAD) from about 0.1 μm to about 3 μm. In some embodiments, the bismuth-thiol composition comprises a plurality of microparticles that comprise amorphous BisEDT, substantially all of said microparticles having a volumetric mean diameter of from about 0.1 μm to about 5 μm, and wherein the BT compound comprises bismuth or a bismuth salt and a thiol-containing compound.

In some embodiments, the present disclosure provides a method of treating, managing or lessening the severity of symptoms and infections associated with one or more pulmonary diseases or infections in a subject, the method comprising administering to the subject a bismuth-thiol (BT) composition that comprises amorphous BisEDT suspended therein, wherein administering the BT composition is via inhalation, orally or nasally, using an aerosol device. In some embodiments, the method is treating, managing or lessening the severity of cystic fibrosis (CF) symptoms and infections in a subject.

In some embodiments, the present disclosure provides a method of treating, managing or lessening the severity of cystic fibrosis (CF) symptoms and infections in a subject, the method comprising administering to the subject a bismuth-thiol (BT) composition that comprises amorphous BisEDT. In some embodiments, the BT composition comprises a plurality of microparticles wherein at least 70%, 80%, or 90% of said microparticles having a volumetric mean diameter (VMD) from about 0.6 μm to about 2.5 μm. In some embodiments, when the BT composition is aerosolized, at least 70%, 80%, or 90% of the aerosolized liquid droplets have a mass median aerodynamic diameter (MMAD) from about 0.9 μm to about 3 μm. In some embodiments, the BT composition comprises BisEDT at a concentration greater than about 0.1 mg/mL, about 0.05% to about 1.0% Tween 80®, about 40 mM to about 250 mM sodium chloride, and optionally about 2 to 20 mM sodium phosphate at about pH. 7.4. In some embodiments, the subject has at least one pulmonary infection containing one or more bacterial pathogens and/or fungal pathogens (as described herein). In some embodiments, the method comprises at least one of: (i) reducing a bacterial biofilm, (ii) impairing growth of a bacterial biofilm, (iii) preventing initial formation of the bacterial biofilm, and/or (iv) preventing reformation of the bacterial biofilm.

### Methods of Treating Wounds

In some embodiments, the present disclosure provides methods for treating a topical wound, comprising administering to a subject in need thereof a therapeutically effective amount of a composition comprising amorphous BisEDT, wherein the composition is applied to the infection (e.g. applied to the surface of the infection).

In some embodiments, the topical wound is a skin ulcer (e.g. a skin ulcer on a lower extremity). In some embodiments, the topical wound is a skin ulcer on a lower extremity, such as the leg and/or foot. In some embodiments, the skin ulcer is one or more of foot ulcer, ischemic ulcer, gangrenous ulcer, venous stasis ulcer, decubitus ulcer, Buruli ulcer, or traumatic ulcer. In some embodiments, the skin ulcer is a foot ulcer. In some embodiments, the topical wound is infected by one or more bacterial and/or fungal pathogens. In some embodiments, the topical wound is infected by bacterial pathogens. In some embodiments, the topical wound is a diabetic foot ulcer. In some embodiments, the diabetic foot ulcer is a diabetic foot infection. In some embodiments, the topical wound is infected with one or more of the following bacterial pathogens: Staphylococcus aureus, MRSA, Escherichia coli, Pseudomonas aeruginosa, Citrobacter spp., Klebsiella oxytoca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingoronas sp., Acinetobacter spp., Anerococcus spp., Dialister spp., Peptoniphilus spp., Finegoldia magna, Peptoniphulus asaccharolyticus, Veillonella atypia, Anaerococcus vaginalis.

Biofilms of S. aureus and other bacteria that are present in the wounds of DFI patients increase the difficulty of successful infection management and reduction. Combinations of DFI-relevant bacteria forming multispecies biofilms containing e.g. S. aureus have demonstrated greater resistance, virulence and pathogenicity than comparable single-species biofilms. The presence of such complex biofilms in the wounds of DFI patients is considered to be largely responsible for the chronic, persistent nature of these infections.

In some embodiments, the bacterial pathogen exhibits resistance to one or more antibiotics. Of particular concern are the methicillin-resistant Staphylococcus aureus strains (MRSA). MRSA remained an uncommon occurrence in hospital setting until the 1990's, when there was an explosion in MRSA prevalence in hospitals. MRSA now is considered endemic to hospitals, especially in the UK (Johnson A P et al. 2001 J. Antimicrobial Chemotherapy 48(1): 143-144). Moreover, MRSA presents a new threat in diabetic foot infections (Retrieved Jan. 17, 2009, from CDC: Centers for Disease Control and Prevention Web site). The ulcers and open sores that can occur in diabetic feet put patients at risk for contracting MRSA, and recent studies show evidence of MRSA impairing healing when present in the diabetic wound (Bowling F L, et al. 2009 Curr Diab Rep 9(6):440-444). See also, Kosinski, M A, et al. 2010. Expert Rev AntiInfect Ther. 8(11):1293-1305. In some embodiments, a bacterial pathogen is resistant to known standards of antibiotic care, including, but not limited to, amikacin, methicillin, vancomycin, nafcillin, gentamicin, metronidazole, Piperacillin/Tazobactam, ampicillin, chloramphenicol, doxycycline, tobramycin, levofloxacin, cephalosporins (e.g. cephalexin, cefoxitin, ceftizoxime, ceftibiprole, ceftazidime, ceftaroline), penicillin/β-lactamase inhibitor combinations (e.g. amoxicillin/clavulanate, ampicillin/sulbactam, piperacillin/tazobactam, and ticarcillin/clavulanate), carbapenems (e.g. imipenem/cilastatin, ertapenem), fluoroquinolones (e.g. ciprofloxacin, moxifloxacin), clindamycin, linezolid, daptomycin, tigecycline, and vancomycin.

Long-term, repeated treatment with antibiotics to treat DFI-associated infections may result in development of antibiotic-resistance, characterized by the presence of microbial biofilms. Recent research has repeatedly demonstrated a correlation between multi-drug resistant (MDR) bacteria, and stronger, more prolific biofilm-forming capabilities. Bacteria within biofilms are protected from antibiotics, which increases the minimal inhibitory concentration of such antibiotics.

The BT compositions (i.e. amorphous BisEDT compositions) of the present disclosure have activity against a plurality of bacterial and fungal strains. In some embodiments, the BT compositions have activity against a plurality of strains including but not limited to Staphylococcus aureus, MRSA, Escherichia coli, Pseudomonas aeruginosa, Citrobacter spp., Klebsiella oxytoca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridiun spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. Accordingly, some embodiments of the present disclosure provide methods of treating and/or preventing infections associated with Staphylococcus aureus. MRSA, Escherichia coli, Pseudomonas aeruginosa, Citrobacter spp., Klebsiella oxytoca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anaerococcus spp., Dialister spp., Peptoniphilus spp., Finegoldia magna, Peptoniphilus asaccharolyticus, Veillonella atypia, Anaerococcus vaginalis in both humans and animals using the BT compositions. In other aspects, the present disclosure provides methods of treating and/or preventing infections associated with related species or strains of these bacteria. In some embodiments, the bacterial infection is an infection associated with diabetic lower extremity infections, such as diabetic foot infections.

Staphylococcus aureus, MRSA, Escherichia coli, Pseudomonas aeruginosa, Citrobacter spp., Klebsiella oxytoca, Proteus spp., Mobiluncus spp., Gardenella spp., Atopibin spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis are responsible for many severe opportunistic infections, particularly in individuals with compromised immune systems, including diabetic patients. The pharmaceutical compositions of the present disclosure are contemplated for treating and/or preventing any infection associated with Staphylococcus aureus, MRSA, Escherichia coli, Pseudomonas aeruginosa, Citrobacter spp., Klebsiella oxytoca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Herophilus spp., Enterococcus spp., Stenotrophomonas spp., Pseudomonas spp., Stenotrophomonas maltophilia, Enterobacter cloacae, Sphingononas sp., Acinetobacter spp., Anerococcus spp., Dialister spp., Peptoniphilus spp., Finegoldia magna, Peptoniphilus asaccharolyticus, Veillonella atypia, Anaerococcus vaginalis or associated with other species or strains of bacteria, including, but not limited to, infections of the skin, infections in and around wounds, chronic ulcers, ulcers associated with burn wounds, post-operative infections, infections associated with catheters and surgical drains, and infections of the blood. In some embodiments, the pharmaceutical compositions of the present disclosure find use in treating and/or preventing bacterial infections associated with areas of non-intact skin, including but not limited to, infections associated with cutaneous ulcers, such as diabetic foot ulcers, skin lesions, vesicles, cysts, blisters, bullae, open sores such as decubitus ulcers (bed sores) and other pressure sores, chronic ulcers, cellulitis and sores associated therewith, erysipelas and lesions associated therewith, wounds, burns and wounds associated therewith, carbuncles, or other conditions where the skin is damaged, cracked, broken, breached, and/or otherwise compromised.

In any of the embodiments described herein, the BT compositions may be used to treat an infection (e.g. DFI) of one or more of the following bacterial pathogens:
Acinetobacter baumanii
Acinetobacter junii
Anaerococcus lactolyticus
Anaerococcus vaginalis
Anaerococcus murdoch
Anaerococcus tetradius
Anaerococcus hydrogenalis
Actinobacidum massiliense
Actinobacuium schaalii
Actinomyces europaeus
Actinomyces honinis
Actinomyces neuii
Actinomyces radingae
Alcaligenes faecalis
Abiotrophia paraadiacens
Bacteroides fragilis
Bulleidia extructa
Bilophila wadsworthia
Campylobacter ureolyticus
Citrobacter murliniae
Clostridium saccharogumia
Clostridium novyi
Corynebacterium accolens
Corynebacterium amycolatum
Corynebacterium aurimucosum
Corynebacterium freiburgense
Corynebacterium hansenii
Corynebacterium jeikeium
Corynebacterium mycetoide
Corynebacterium simulans
Corynebacterium tuberculostearicum
Corynebacterium xerosis
Corynebacterium striatum
Dermabacter hominis
Dialister invisus
Dialister propionicifaciens
Dialister micraerophilus
Dialister pneumosintes
Delftia acidovorans
Enterobacter aerogenes
Enterobacter cloacae
Enterobacter hormaechei
Enterococcus faecalis
Enterococcus canintestini
Escherichia coli
Escherichia fergusonii
Escherichia vulneris
Enterococcus avium
Enterococcus gallinarum
Enterococcus casseliflavus
Eggerthella lenta
Finegoldia magna
Fusobacterium canifelinum
Fusobacterium nucleatum
Fusobacterium periodontium
Granulicatella adiacens
Gemella morbillorum
Globicatella sanguinis
Haemophilus parainfluenzae
Haemophilus segnis
Helcococcus kunzii
Helcococcus kunzii
Klebsiella oxytoca
Kocuria atrinae
Leclercia adecarboxylata
Mobiluncus curtisii
Moryella indoligenes
Morganella morganii
Negativicoccus succinicivorans
Peptoniphilus harei
Peptoniphilus gorbachii
Peptoniphilus ivorii
Peptoniphilus lacrimalis
Peptoniphilus olsenii
Peptoniphilus asacchrolyticus
Parvimonas micra
Peptococcus niger
Peptostreptococcus anaerobius
Peptostreptococcus stomatis
Porphyromonas asaccharolytica
Porphyromonas bennonis
Porphyromonas somerae
Porphyromonas uenonis
Porphyromonas levii
Prevotella timonensis
Prevotella bergensis
Prevotella buccalis
Prevotella corporis
Prevotella disiens
Prevotella intermedia
Prevotella nanceiensis
Pseudomonas indica
Pseudomonas otitidis
Psychrobacter lutiphocae
Proteus myxofaciens
Proteus hauseri
Providencia rettgeri
Providencia stuartii
Staphylococcus aureus
Staphylococcus epidermidis
Staphylococcus carnosus
Staphylococcus chromogens
Staphylococcus devriesei
Staphylococcus hominis
Staphylococcus lugdunensis
Serratia nematodiphila
Stenotrophomonas maltophilia
Staphylococcus pettenkoferi
Staphylococcus capitis
Staphylococcus saprophyticus
Streptococcus agalactiae
Streptococcus anginosus
Streptococcus canis
Streptococcus dysgalactiae
Streptococcus infantarius
Streptococcus oralis
Serratia grimesii
Stenotrophomonas pavanni
Salmonella enterica
Trueperella bernardiae
Varibaculum cambriense
Veillonella atypica
Veillonella parvula
Veillonella dispar
Veillonella rogosae
Acinetobacter calcoaceticus
Acinetobacter lwoffi
Anaerococcus prevotii
Bacteroides caccae
Bacteroides distasonis
Bacteroides ovatus
Bacteroides stercoris
Bacteroides thetaiotaomicron
Bacteroides uniformis
Bacteroides vulgatus
Corynebacterium striatum
Clostridium innocuum
Clostridium perfringens
Clostridium ramosum
Pluralibacter gergoviae
Fusobacterium mortiferum
Finegoldia magna
Klebsiella oxytoca
Klebsiella pneumoniae
Pseudomonas aeruginosa
Peptococcus magnus
Prevotella bivia
Prevotella melaninogenica
Porphyromonas asaccharolytica
Peptostreptococcus asaccharolyticus
Peptostreptococcus micros
Parvimonas micra
Proteus mirabilis
Staphylococcus haemolyticus
Staphylococcus simulans
Staphylococcus saprophyticus
Streptococcus pneumoniae
Streptococcus agalactiae
Streptococcus mitis
Streptococcus milleri
Streptococcus dysgalactiae
Streptococcus canis
Serratia marcescens
Serratia liquefaciens
Stenotrophomonas maltophila
Epidermolysis bullosa

In any of the embodiments described herein, the BT compositions may be used to treat an infection (e.g. DFI) of one or more of the following fungal pathogens: Candida spp., Cladosporium spp., Aspergillus spp., Penicillium spp., Alternaria spp., Pleospora spp., Fusarium spp., Candida lusitaniae, Candida parapsilisis, and Candida albicans.

In some embodiments, the BT compositions of the present disclosure find use in treating chronic ulcers. Chronic ulcers may arise from wounds caused by a variety of factors, especially in patients with impaired blood circulation, for example, caused by cardiovascular issues or external pressure from a bed or a wheelchair. More than 8 million patients are diagnosed with chronic skin ulcers each year in the United States alone (Harsha, A. et al., 2008, Journal of Molecular Medicine, 86(8): 961-969), which costs more than 10 billion dollars per year (Margolis, D J, et al., 2002, Journal of the American Academy of Dermatology 46(3): 381-386). Chronic ulcers may develop in the mouth, throat, stomach, and skin. Chronic skin ulcers include diabetic ulcers, venous ulcers, radiation ulcers, and pressure ulcers, the three major categories of chronic skin ulcers being diabetic ulcers, venous stasis ulcers, and pressure ulcers. Chronic ulcers can cause the loss of the integrity of large portions of the skin, even leading to morbidity and mortality.

In some embodiments, the BT compositions of the present disclosure find use in treating diabetic lower extremity infections, such as diabetic foot infections. Diabetic foot infection is one of the major complications of diabetes mellitus, occurring in about 15% of all diabetic patients and resulting in about 85% of all lower leg amputations. (Brem, et al., J. Clinical Invest., 2007, 117(5):1219-1222). Diabetes mellitus impedes the normal steps of the wound healing process, such that diabetic foot infections can become associated with non-healing, chronic cutaneous ulcers.

A chronic wound represents a failure of the normal processes of acute wound healing. Wound healing has traditionally been divided into three distinct phases: inflammation, proliferation and remodeling. The inflammatory phase of wound healing begins at the time of injury by forming a clot via a platelet plug, thereby initiating a response from neutrophils and macrophages. Neutrophils initially clear the wound of bacteria and debris by releasing a variety of proteases and reactive oxygen free radicals. Macrophages are then attracted to the wound site by chemoattractants and subsequently release their own chemoattractants to stimulate fibroblasts and more macrophages. During the proliferation phase, fibroblasts initiate epithelialization, angiogenesis, and collagenation. Epithelialization generally occurs from the basement membrane if it remains intact and from the wound margins if not intact. Fibroblasts synthesize type II collagen during this phase and transform into myofibroblasts, which help to stimulate wound contraction. During the remodeling phase, type III collagen begins to be replaced by type 1 collagen. Collagen is woven into an organized, cross-linked network whose strength approaches 80% of the original uninjured tissue.

There are many factors that can stall the three-phase healing process and convert an acute wound into a chronic wound. These may include a low proliferative capacity of the fibroblasts, downregulation of receptors, reduced growth factors, or the absence of a suitable protein matrix in the dermis. Further, poor perfusion and/or nutrition can cause a wound to halt in the inflammatory phase and lead to excessive build-up of exudate in the wound. A chronic ulcer can be considered to be a non-healing area of non-intact skin, such as an area of non-intact skin that fails to follow the normal processes of wound healing, e.g., as described above, and/or that fails to respond, or fails to respond appropriately, to initial treatment. A chronic ulcer on the skin may be characterized as a wound lesion lasting more than four weeks, without remarkable healing tendency or as a frequently recurrent wound (Fonder, M. et al., 2012, Journal of the American Academy of Dermatology 58(2): 185-206). A chronic wound may appear with red granulation and yellow pus, a dim purple skin around granular tissues, or gray-white and swelling granulation. Standard care procedures for chronic skin ulcer include, e.g., the following: removal of necrotic or infected tissue; establishment of adequate blood circulation; maintenance of a moist wound environment; management of wound infection; wound cleansing; and nutritional support, including blood glucose control for subjects with diabetic ulcers. For example, in the diabetic patient, poor control of blood glucose levels allows bacteria to grow more rapidly in a wound; further still, neural degeneration in diabetes means the condition may not be painful and thus go undetected, at least initially. Chronic ulcers, including diabetic foot ulcers, often become further infected with opportunistic bacteria, leading to exacerbation of the condition. Staphylococcus aureus, MRSA, Escherichia coli, Pseudomonas aeruginosa, Citrobacter spp., Klebsiella oxytoca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dvsgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis are associated with such infections.

In some embodiments, the pharmaceutical composition of the present disclosure is formulated for use in methods of treating and/or preventing bacterial infections caused by Staphylococcus aureus, MRSA, Escherichia coli, Pseudomonas aeruginosa, Citrobacter spp., Klebsiella oxytoca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some embodiments, the pharmaceutical composition of the present disclosure is formulated for use in methods of treating and/or preventing bacterial infections caused by Staphylococcus aureus, MRSA, Escherichia coli, Pseudomonas aeruginosa, Citrobacter spp., Klebsiella oxyloca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalaciae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some other embodiments, the pharmaceutical composition of the present disclosure is formulated for use in methods of treating and/or preventing bacterial infections caused by bacteria other than Staphylococcus aureus, MRSA, Escherichia coli, Pseudomonas aeruginosa, Citrobacter spp., Klebsiella oxytoca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis.

In some embodiments, the present disclosure provides methods of treating and/or preventing chronic ulcers, comprising administering to a subject in need thereof a therapeutically or prophylactically effective amount of a pharmaceutical composition of the present disclosure. In some embodiments, administration comprises topical administration to the area of non-intact skin associated with the chronic ulcer. In some embodiments, topical administration follows debridement of the area to be treated

In some embodiments, the present disclosure provides methods of treating and/or preventing diabetic foot infections, comprising administering to a subject in need thereof a therapeutically or prophylactically effective amount of a pharmaceutical composition of present disclosure. In some embodiments, administration comprises topical administration to the area of non-intact skin associated with the diabetic foot infection, e.g., a cutaneous ulcer. In some embodiments, topical administration follows debridement of the area to be treated.

Debridement can be accomplished by a number of approaches. Surgical debridement involves cutting away dead tissues of the wound or other area of non-intact skin. Mechanical debridement uses various methods to loosen and remove wound debris, such as a pressurized irrigation device, a whirlpool water bath, ultrasound, larval maggots, or specialized dressings. Autolytic debridement enhances the body's natural process of recruiting enzymes to break down dead tissue, for example, using an appropriate dressing that keeps the wound moist and clean. Enzymatic debridement uses chemical enzymes and appropriate dressings to further aid in the break down dead tissues at the site of a wound or other area of non-intact skin.

Debridement improves topical treatment because it reduces the bioburden of bacteria present and also opens a time-dependent therapeutic window for topical antimicrobial therapy (TAT) (Wolcott R D, et al. 2010. J Wound Care 19:320-328). Regarding the timing for debridement, early or immediate debridement is preferred to delayed debridement once this treatment option is chosen in the management of a wound. Further, multiple debridements during wound management may be indicated (Wolcott R D, et al. 2009. J Wound Care 18(2):54-6). For example, in some embodiments, debridement precedes topical application of a BT composition of the present disclosure, and is repeated before every administration of the BT composition. In some embodiments, debridement is performed only before every other administration of the BT composition, or only before every 3^{rd}, 4^{th}, 5^{th}, or 6^{th} administration of the BT composition. In some embodiments, debridements are performed less frequently than the application of the BT composition, for example, once a week. Accordingly, if the BT composition is applied daily, the patient will not get debridement every time it is applied. In some embodiments, whether or not wound debridement is performed before topical administration of a BT composition of the present disclosure is within the clinical judgment of a health care practitioner treating the wound, e.g., the physician, physician's assistant, or emergency medical personnel.

The BT compositions of the present disclosure can find use in the treatment, management, control, and/or prevention of infections associated with chronic ulcers, including diabetic foot infections and cutaneous ulcers associated therewith. In other embodiments, BT compositions of the present disclosure find use in the treatment, management, control, and/or prevention of microbial (e.g. bacterial and/or fungal) infections associated with other areas of non-intact skin, such as a cellulitis sore, an erysipelas lesion, a decubitus ulcer, a burn wound, a traumatic wound, and a pressure sore. In some such embodiments, the composition used may be a topical composition, formulated for topical administration, e.g., for direct application to an area of non-intact skin, such as described above.

The BT compositions of the present disclosure also find use in the treatment, management, control, and/or prevention of decubitus ulcers. Decubitus ulcers, also called pressure sores or pressure ulcers, are injuries to the skin and underlying tissues resulting from prolonged pressure on the area. For example, bedsores most often develop on skin that covers bony areas of the body, such as the heel, ankles, hips or buttocks.

Bedsores fall into one of four stages based on their severity. Stage I is the beginning stage of a pressure sore while the skin still is intact. The skin may appear red, ashen, bluish or purple, and fails to blanch when touched. Stage II often involves an open wound of non-intact skin. At this stage, the outer layer of skin (epidermis) and part of the underlying layer of skin (dermis) has been damaged or lost. The ulcer may appear as a shallow, pinkish-red, basin-shaped wound. In Stage III, the ulcer is a deep wound, where the loss of skin may expose some amount of fat, and the ulcer has a crater-like appearance. The bottom of the wound also may have some yellowish dead tissue (slough). A Stage IV ulcer exhibits large-scale loss of tissue, where the wound may expose muscle, bone and tendons. The bottom of the wound will likely contain slough or dark, crusty, dead tissue (eschar).

As in the treatment of diabetic foot ulcers, debridement (e.g. of pressure ulcers or bedsores) may be used to remove damaged, dead, or infected tissue from the wound, facilitating proper healing, e.g., as described herein and/or otherwise known in the art. In some embodiments, administration of a pharmaceutical composition of the present disclosure follows debridement. For example, a pharmaceutical composition disclosed herein may be topically administered to a decubitus ulcer following surgical, mechanical, autolytic, or enzymatic debridement thereof.

BT compositions of the present disclosure also find use in the treatment, management, control, and/or prevention of cellulitis and/or erysipelas, including but not limited to sores and lesions associated with cellulitis and erysipelas. Cellulitis and erysipelas are skin infections that develop as a result of bacterial entry via breaches in the protective barrier of the skin. For example, cracks in the skin, cuts, blisters, burns, insect bites, spider bites, tattoos, surgical wounds, intravenous drug injection, or sites of intravenous catheter insertion may provide a means of entry for bacteria. Group A Streptococcus and Staphylococcus are the most common bacteria involved in cellulitis. Cellulitis is observed most frequently among middle-aged and elderly individuals, while erysipelas occurs in young children and the elderly (Ellis Simonsen S M et al. 2006. Epidemiol Infect. 134(2):293; and Eriksson B. et al. 1996 Clin Infect Dis 23:1091). Also, people with immune deficiency, diabetes, alcoholism, fungal infections, and impaired lymphatic drainage are at increased risk. Diabetics are especially prone to cellulitis in the feet, because the disease causes impairment of blood circulation in the legs. The lower extremities are the most common site of infection for both erysipelas and cellulitis (Ellis Simonsen S M et al. 2006. Epidemiol Infect. 134(2):293; Chartier C et al 1996 Int J Dermatol 35:779).

Cellulitis and erysipelas often coexist and generally manifest as areas of skin erythema, edema, and warmth. They differ in that erysipelas involves the upper dermis and superficial lymphatics, whereas cellulitis involves the deeper dermis and subcutaneous fat. Accordingly, erysipelas has more distinctive anatomic features than cellulitis-erysipelas lesions may be raised above the level of surrounding skin with a clear line of demarcation between involved and uninvolved tissue (Bisno A L et al. 1996 N Engl J Med 334:240). The lesion may appear red, swollen, warm, hardened, and/or as a rash similar in consistency to an orange peel. Erysipelas may appear on the face, for example, in a “butterfly” pattern. More severe infections can result in vesicles, bullae, and petechiae, with possible skin necrosis. In addition, patients with erysipelas tend to have acute onset of signs and symptoms with systemic manifestations, including fever and chills.

Patients with cellulitis tend to have a more gradual course of development, with signs and symptoms appearing over a few days' time. Various forms of cellulitis include periorbital cellulitis, abdominal wall cellulitis (in morbidly obese individuals), buccal cellulitis (due to Streptococcus pneumoniae), Ludwig's angina (cellulitis within the submandibular space), and perianal cellulitis (due to group A beta-hemolytic streptococcus) (Barzilai A, et al, 1998 Pediatr Infect Dis J. 17(4):358; Thorsteinsdottir B, et al. 2005 Scand J Infect Dis. 37(8):605). Cellulitis also can result in influenza-like signs and symptoms, with high temperatures and shaking.

In some embodiments, treatment of cellulitis or erysipelas further comprises administration of an antibiotic agent. For example, a pharmaceutical composition according to the present disclosure may be topically administered to an erysipelas lesion, in combination with an antibiotic agent selected from the group consisting of penicillin, clindamycin, and erythromycin. As another example, a pharmaceutical composition according to the present disclosure may be topically administered to a sore associated with cellulitis, in combination with an antibiotic agent selected from the group consisting of flucloxacillin, dicloxacillin, penicillins, ampicillin, and amoxicillin. The antibiotic may be administered orally, intravenously, or topically, e.g., along with topical administration of a BT composition of the present disclosure.

BT compositions of the present disclosure also find use in the treatment, management, control, and/or prevention of infections associated with burn wounds. A burn wound is any area of non-intact skin caused, directly or indirectly, from a burn. A burn is a type of injury to the skin that can be caused by heat, as well as electricity, chemicals, light, radiation or friction. Burns may affect only the skin (epidermal tissue), but in some cases also injure deeper tissues, such as muscle, bone, and blood vessels. Burns can be classified by mechanism of injury, depth, extent and associated injuries, and comorbidities. Burns conventionally are described based on the depth of injury to the dermis, being loosely classified as first, second, third, and fourth degree burns (Walls et al., 2009, Rosen's Emergency Medicine: Expert Consult Premium Edition (Rosen's Emergency Medicine: Concepts & Clinical Practice (2v)). Important characteristics of a burn wound include its cause (thermal, chemical, electrical), anatomic location, depth (full or partial thickness), duration, and extent (percent total body surface area). Patient characteristics that affect burn wound healing include age, nutritional status, underlying medical conditions, and concomitant injury (e.g., head trauma, inhalation injury, bone fractures).

Infections among burn patients are a major problem, with the reported incidence of nosocomial infections varying at 63-240 per 100 patient days and 53-93 per 1000 patient days, mainly depending on the definitions used (Chim H, et al, 2007, Burns 33:1008-1014; and Wibbenmeyer L, et al., 2006, J Burn Care Res 27:152-60). Moreover, bacterial infection of burn wounds are associated with adverse outcomes and mortality. In a series of 175 patients with severe burns, for example, infections preceded multiorgan dysfunction in 83% of patients and were considered the direct cause of death in 36% of patients who did not survive (Fitzwater J, et al., 2003, J Trauma 54:959-66). Burn wounds may become infected from multiple sources. Burn wounds may become initially infected with Gram positive bacteria, mainly staphylococci, that are normal deep inhabitants of the sweat glands and hair follicles exposed by the burn (Sharma B R., 2007, Infect Dis Clin North Am 21:745-59; ix). The moist, vascular burn eschar further may foster microbial growth. Gram negative bacterial infections may result from translocation from the colon, for example, due to reduced mesenteric blood flow at the time of burn and subsequent insults (Herndon D N, et al., 2000, Crit Care Med 28:1682-3). Furthermore, burn patients may develop immune deficits, including impaired cytotoxic T lymphocyte response, myeloid maturation arrest causing neutropenia, impaired neutrophil function, and decreased macrophage production (Sharma B R., 2007, Infect Dis Clin North Am 21:745-59, ix; Gamelli R L, et al., 2000, J Burn Care Rehabil 21:64-9; Hunt J P, et al., 1998, J Surg Res 80:243-51; and Shoup M, et al., 1998, Ann Surg 228:112-22). Finally, burn patients are susceptible to hospital acquired infections, common to other patients in intensive care units, including intravascular catheter related infections and ventilator associated pneumonia, with an overall incidence of infection higher than that of other patients in intensive care units (Chim H, et al., 2007, Burns 33:1008-14; and Wibbenmeyer L, et al., 2006, J Burn Care Res 27:152-60). Indeed, most episodes of bloodstream infection in burn patients after the first week are caused by hospital-type multidrug resistant bacteria (Wibbenmeyer L, et al., 2006, J Burn Care Res 27:152-60; and Ressner R A, et al., 2008, J Am Coll Surg 206:439-44).

Conventional treatment of burns includes debridement and excision, applying dressings the wound, wound closure, skin grafting, fluid resuscitation, management of wound infection such as administering antibiotics, pain control, nutritional support, and/or measures to inhibit excessive scar formation. A burn may be covered with a clean and dry sheet or dressing (such as cling film). Early cooling with cool water, within 30 minutes of the burn, reduces burn depth and pain. Debridement, cleaning, and dressings are important aspects of burn wound care.

In some embodiments, treatment of a burn wound further comprises administration of an antibiotic agent. It has been shown that antibiotic prophylaxis may reduce mortality, bacteraemia, and ventilator associated pneumonia among patients in intensive care units (Silvestri L, et al, 2007, J Hosp Infect 65:187-203; and De Smet A M, et al., 2009, N Engl J Med 360:20-31). In burns patients, the skin is an additional source of infection (Avni T, et al., 2010, BMJ 340: c241). In some embodiments, treatment of a burn wound further comprises administration of an agent for managing pain. A pharmaceutical composition according to the present disclosure may be topically administered to a burn wound, in combination with an agent for pain management selected from the group consisting of a simple analgesic, ibuprofen, acetaminophen, and a narcotic. The antibiotic agent and/or agent for managing pain may be administered orally, intravenously, or topically, e.g., along with topical administration of a BT composition of the present disclosure. One or more other aspects of conventional treatment of burns also may be used in combination with a BT composition of the present disclosure.

In some embodiments, the agent for pain management for use in combination with the BT composition of the present disclosure includes one or more agents selected from the group consisting of: paracetamol (acetaminophen), a non-steroidal anti-inflammatory drug, ibuprofen, ketoprofen, piroxicam, hydrocodone, morphine, hydromorphine, oxymorphone, fentanyl, oxycodone, diamorphine, methadone, buprenorphine, meperidine, pentazocine, dextromoramide, dipipanone, amitriptyline, dilaudid, tapentadol, and methadone. The agent for pain management may include any other agent for pain described herein and/or known in the art.

In some embodiments, the agent for pain management is one that can be applied topically, such as a topical anesthetic agent. A topical anesthetic agent is a local anesthetic agent that is used to numb the surface of a body part, such as any area of the skin, the front of the eyeball, the inside of the nose, ear or throat, the anus, or the genital area. In some embodiments, the agent for pain management for use in combination with a BT composition of the present disclosure includes one or more topical anesthetic agents selected from the group consisting of benzocaine, butamben, dibucaine, lidocaine, oxybuprocaine, pramoxine, prilocaine, proparacaine, proxymetacaine, and tetracaine (amethocaine). Topical anesthetic agents are available in creams, ointments, aerosols, sprays, lotions, and jellies. In further embodiments, the topical anesthetic agent may be used with one or more additional agents for pain management, such as another topical anesthetic agent, or a different agent for pain management, such as any other agent(s) for pain management described herein and/or known in the art.

BT compositions of the present disclosure will comprise a therapeutically and/or prophylactically effective amount of one of more BT compounds (e.g. amorphous BisEDT), as described herein. A therapeutically and/or prophylactically effective amount refers to an amount required to bring about a therapeutic and/or prophylactic benefit, respectively, in a subject receiving said amount. A therapeutically and/or prophylactically effective amount will depend on the particular formulation, route of administration, condition being treated, whether other agents or therapies are used in combination with methods of the present disclosure, and other factors.

In some embodiments of the methods for treating a topical wound, the subject experiences one or more of the following outcomes following the completion of dosing:
the wound is healed or substantially healed within 12 weeks of the first administration of the composition; and/or
the prevention of amputation and/or infection-related surgery; and/or
the wound is closed partially or fully; and/or
the wound is reduced in size from about a 1% reduction relative to the original wound size to total elimination of the wound; and/or
the wound is 30 days old or greater and is healed or substantially healed.
   In some embodiments, the subject experiences two or more of the recited outcomes. In some embodiments, the subject experiences three or more of the recited outcomes. In some embodiments, the subject experiences four or more of the recited outcomes. In some embodiments, the subject experiences all of the recited outcomes. In some embodiments of the methods for treating a topical wound, the subject experiences one or more of the following outcomes following the completion of dosing: less reinfection/relapse for the 12 weeks after start of treatment; resolution or improvement in signs and/or symptoms of infection that include redness, swelling, induration, exudate, pain, warmth (at site of infection) or fever; improved quality of life; eradication of insulting pathogens and/or biofilm; reduced need for concurrent systemic antibiotics.

In some embodiments, administration of a therapeutically effective amount of a BT composition, in accordance with the present disclosure, results in improved wound closure (partial or full), such as a reduction in the area of non-intact skin (wound area) compared to the area before initiation of treatment. Wound area can be expressed as a percentage of the initial wound area, at one or more time points after initiation of treatment. For example, in some embodiments, wound area decreases by at least about 20%, at least about 30%, at least about 40%, at least about 50%, at least about 60%, at least about 70%, at least about 80%; or at least about 90% over a course of treatment with a BT composition of the present disclosure. In some specific embodiments, the decrease in wound area occurs at least by day 1 after treatment initiation (t1), day 2 after treatment initiation (t2), day 3 after treatment initiation (t3), day 4 after treatment initiation (t4), day 5 after treatment initiation (t5), day 6 after treatment initiation (t6), day 7 after treatment initiation (t7), day 8 after treatment initiation (t8), day 9 after treatment initiation (t9), day 10 after treatment initiation (t10), day 12 after treatment initiation (t12), day 15 after treatment initiation (t15), day 20 after treatment initiation (t20), day 25 after treatment initiation (t25), or day 30 after treatment initiation (t30).

In some embodiments, administration of a therapeutically effective amount of a BT composition, in accordance with the present disclosure, results in improved wound healing, such as an improvement in ulcer grade based on the PEDIS classification compared to the ulcer grade before initiation of treatment. PEDIS is a routinely used, validated classification system for infections associated with wounds that has been developed by the International Working Group on the Diabetic Foot (IWGDF). IWGDF specifically developed a system for classifying wounds associated with diabetic foot infections that uses the acronym PEDIS, which stands for perfusion, extent (size), depth (tissue loss), infection, sensation (neuropathy). The classification originally was developed as a research tool (Schaper N C., 2004, Diabetes Metab Res Rev 20(Suppl 1):S90-5), and offers a semi-quantitative gradation for the severity of each of the categories. Specifically, PEDIS Grade 1 corresponds to no symptoms or signs of infection; Grade 2 corresponds to a local infection involving only the skin and subcutaneous tissue (without involvement of deeper tissues and without systemic signs), while any erythema involved must be between 0.5 cm and 2 cm; Grade 3 corresponds to a local infection, as described for Grade 2, but involving an erythema of greater than 2 cm or involving structures deeper than skin and subcutaneous tissues (e.g., abscess, osteomyelitis, septic arthritis, fasciitis), but without any systemic inflammatory response signs; and Grade 4 corresponds to a local infection, as described for Grades 2 and 3, but with the signs of systemic inflammatory response syndrome, as manifested by more than two of the following: a temperature >38° C. or <36° C.; a heart rate >90 beats/min; a respiratory rate >20 breaths/min or partial pressure of arterial carbon dioxide <32 mm Hg; and a white blood cell count >12000 or <4000 cells/μL or >10% immature (band) forms (see, e.g., Lipsky, B A, et al., 2012, CID 54:e132-e173). Another classification system has been developed by the IDSA (the Infectious Diseases Society of America), which rates the infection severity of infected wounds, in particular, diabetic foot infections. Specifically, the IDSA rates PEDIS Grades 1-4 as “uninfected”, “mild”, “moderate”, and “severe”, respectively (see, again, Lipsky, B A, et al., 2012, CID 54:e132-e173).

In some embodiments, the PEDIS grade decreases from Grade 4 to Grade 3, Grade 2, or Grade 1, over a course of treatment with a BT composition of the present disclosure. In some embodiments, the PEDIS grade decreases from Grade 3 to Grade 2 or Grade 1, over a course of treatment with a BT composition of the present disclosure. In some embodiments, the PEDIS grade decreases from Grade 2 to Grade 1 over a course of treatment with a BT composition of the present disclosure. In some embodiments, the decrease in ulcer grade occurs by at least day 1 after treatment initiation (t1), day 2 after treatment initiation (t2), day 3 after treatment initiation (t3), day 4 after treatment initiation (t4), day 5 after treatment initiation (t5), day 6 after treatment initiation (t6), day 7 after treatment initiation (t7), day 8 after treatment initiation (t8), day 9 after treatment initiation (t9), day 10 after treatment initiation (t10), day 12 after treatment initiation (t12), day 15 after treatment initiation (t15), day 20 after treatment initiation (t20), day 25 after treatment initiation (t25), day 30 after treatment initiation (t30), day 35 after treatment initiation (t35), day 40 after treatment initiation (t40), day 45 after treatment initiation (t45), day 50 after treatment initiation (t50), day 55 after treatment initiation (t55), day 60 after treatment initiation (t60), day 65 after treatment initiation (t65), day 70 after treatment initiation (t70), day 75 after treatment initiation (t75), day 80 after treatment initiation (t80), or day 85 after treatment initiation (t85). In some embodiments, the decrease in ulcer grade occurs after day 85 after treatment initiation (t85+).

In some embodiments of the methods for treating a topical wound, the BT composition comprises amorphous BisEDT as a suspension of microparticles having a volumetric mean diameter (VMD) from about 0.4 μm to about 5 μm.

In some embodiments of the methods for treating a topical wound, the applied amorphous BisEDT is present on the surface at a concentration greater than about 20 μg/cm². In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments of the methods for treating a topical wound, after administration of the BT composition, one or more of the following occurs: (i) reducing and or dispersing a microbial (e.g. bacterial and/or fungal) biofilm, (ii) impairing growth or formation of a microbial (e.g. bacterial and/or fungal) biofilm, and (iii) preventing reformation or spread of a microbial (e.g. bacterial and/or fungal) biofilm. In some embodiments, the BT composition treats, manages, and/or lessens the severity of the diabetic foot infection by one or both of: (i) prevention of the infection by the bacterial or fungal pathogen; and/or (ii) reduction of the bacterial or fungal pathogen. In some embodiments, the BT composition treats, manages or lessens the severity of the infection by one or more of: (i) prevention of elaboration or secretion of exotoxins from the bacterial or fungal pathogen; (ii) inhibition of cell viability or cell growth of planktonic cells of the bacterial or fungal pathogen; (iii) inhibition of biofilm formation by the bacterial or fungal pathogen; (iv) inhibition of biofilm or microbial pathogen invasiveness to underlying tissues (e.g. subcutaneous tissue); (v) inhibition of biofilm or microbial pathogen pathogenicity to underlying tissues (e.g. subcutaneous tissue); (vi) inhibition of biofilm viability or biofilm growth of biofilm-forming cells of the bacterial or fungal pathogen; and/or (vii) prevents the reformation of biofilm after debridement.

In some embodiments, the infection contains one or more bacterial or fungal pathogens. In some embodiments, the disclosed methods comprise treating the DFI-related infection. In some embodiments, the disclosed methods comprise managing the DFI-related infection. In some embodiments, the disclosed methods comprise lessening the severity of the DFI-related infection.

In some embodiments, the bismuth-thiol composition comprises a plurality of microparticles that comprise amorphous BisEDT, substantially all of said microparticles having a volumetric mean diameter of from about 0.4 μm to about 5 μm. In some embodiments, at least 70% of the microparticles have a volumetric mean diameter of from about 0.4 μm to about 3 μm, or from about 0.5 μm to about 2 μm, or from about 0.7 μm to about 2 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, or any narrow ranges between the specific ranges described above.

In some embodiments of the methods for treating a topical wound, the administered amorphous BisEDT is present on the surface at a concentration from about 1 μg/cm² to about 1,000,000 μg/cm² (e.g. about 1 μg/cm² to about 10,000 μg/cm²), including all integers and ranges therebetween. In some embodiments, the amorphous BisEDT composition is present on the surface at a concentration from about 50 μg/cm² to about 200 μg/cm². In some embodiments, the applied amorphous BisEDT is present on the surface at a concentration from about 250 μg/cm² to about 5,000 μg/cm². For example, in some embodiments, amorphous BisEDT is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, amorphous BisEDT is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments, the present disclosure provides methods of treating or preventing a bacterial infection in a subject in need thereof comprising administering to the subject a therapeutically effective amount of the pharmaceutical composition as described herein (i.e. an amorphous BisEDT-containing composition). In some embodiments, the bacterial infection is an infection by one or more of Staphylococcus aureus, MRSA, Escherichia coli, Pseudomonas aeruginosa, Citrobacter spp., Klebsiella oxytoca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some embodiments, the pharmaceutical composition is administered topically. In some embodiments, the subject is a mammal, e.g., a human. In some embodiments, the bacterial infection is diabetic foot infection. In some embodiments, the diabetic foot infection comprises a cutaneous ulcer. In some embodiments, the bacterial infection is associated with an area of non-intact skin selected from a sore associated with cellulitis, an erysipelas lesion, a burn wound, a chronic ulcer, a decubitus ulcer, and a pressure sore. In some embodiments, the treatment comprises topically administering the pharmaceutical composition to a cutaneous ulcer associated with diabetic foot infection. In some embodiments, administration follows mechanical debridement of the ulcer. In some embodiments, administration comprises use of at least one of a dressing, an instillation device, and a negative pressure wound therapy device.

In some embodiments of the methods for treating a topical wound, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments of the methods for treating a topical wound, the wound area is from about 0.1 cm² to about 250 cm², including all integers and ranges therebetween. For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm², about 160 cm², about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides methods for treating a bacterial infection, comprising administering to a subject in need thereof a therapeutically effective amount of a composition comprising amorphous BisEDT, wherein the composition is applied to the infection (e.g. applied to the surface of the infection). In some embodiments, the bacterial infection is a diabetic foot infection. In some embodiments, the bacterial infection comprises one or more of the following bacterial pathogens: Staphylococcus aureus, MRSA, Escherichia coli, Pseudomonas aeruginosa, Citrobacter spp., Klebsiella oxytoca, Proteus spp., Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis.

In some embodiments of the methods for treating a microbial (e.g. bacterial and/or fungal) infection, the infection is associated with a wound (e.g. an ulcer) and the subject experiences one or more of the following outcomes following the completion of dosing:
the wound is healed or substantially healed within 12 weeks (e.g. within 4 weeks) of the first administration of the composition; and/or
the prevention of amputation and/or infection-related surgery, and/or
the wound is closed partially or fully; and/or
the wound is reduced in size from about a 1% reduction relative to the original wound size to total elimination of the wound; and/or
the wound is 30 days old or greater and is healed or substantially healed.
   In some embodiments, the subject experiences two or more of the recited outcomes. In some embodiments, the subject experiences three or more of the recited outcomes. In some embodiments, the subject experiences four or more of the recited outcomes. In some embodiments, the subject experiences all of the recited outcomes.

In some embodiments of the methods for treating a microbial (e.g. bacterial and/or fungal) infection, the BT composition is a suspension of microparticles comprising amorphous BisEDT having a volumetric mean diameter (VMD) from about 0.01 μm to about 5 μm. In some embodiments, at least 70% of the microparticles have a volumetric mean diameter of less than about 5 μm, less than about 4 μm, less than about 3 μm, less than about 2 μm, less than about 1 μm, from about 0.01 μm to about 5 μm, from about 0.1 μm to about 5 μm, of from about 0.4 μm to about 3 μm, or from about 0.5 μm to about 2 μm, or from about 0.7 μm to about 2 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, or any narrow ranges between the specific ranges described above.

In some embodiments of the methods for treating a microbial (e.g. bacterial and/or fungal) infection, the BT composition comprises amorphous BisEDT and which is present on the surface at a concentration greater than about 20 μg/cm².

In some embodiments of the methods for treating a microbial (e.g. bacterial and/or fungal) infection, the BT composition further comprises about 0.05% to about 1.0% Tween 80®, about 0.05 to 40 mM sodium chloride, optionally about 1% to about 10⁰% of methylcellulose, and optionally about 2 to 20 mM sodium phosphate at about pH. 7.4.

In some embodiments of the methods for treating a microbial (e.g. bacterial and/or fungal) infection, the method comprises at least one of: (i) reducing and or dispersing a microbial (e.g. bacterial and/or fungal) biofilm, (ii) impairing growth or formation of a microbial (e.g. bacterial and/or fungal) biofilm, and (iii) preventing reformation or spread of a microbial (e.g. bacterial and/or fungal) biofilm. In some embodiments, the BT composition treats, manages or lessens the severity of the diabetic foot infection by one or both of: (i) prevention of the infection by the bacterial or fungal pathogen; and (ii) reduction of the bacterial or fungal pathogen. In some embodiments, the BT composition treats, manages or lessens the severity of the infection by one or more of: (i) prevention of elaboration or secretion of exotoxins from the bacterial or fungal pathogen; (ii) inhibition of cell viability or cell growth of planktonic cells of the bacterial or fungal pathogen; (iii) inhibition of biofilm or microbial pathogen formation by the bacterial or fungal pathogen; (iv) inhibition of biofilm invasiveness to underlying tissues (e.g. subcutaneous tissue); (v) inhibition of biofilm or microbial pathogen pathogenicity to underlying tissues (e.g. subcutaneous tissue); (vi) inhibition of biofilm viability or biofilm growth of biofilm-forming cells of the bacterial or fungal pathogen; and/or (vii) prevents the reformation of biofilm after debridement.

In some embodiments of the methods for treating a microbial (e.g. bacterial and/or fungal) infection, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration from about 250 μg/cm² to about 5,000 μg/cm². For example, in some embodiments, the bismuth thiol compound in the BT composition is amorphous BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In another embodiment, the dose volume may range from about 0.01 mL to about 10 mL or any range therein between. In another embodiment, the dose volume may range from about 0.1 mL to about 1 mL or any range therein between. In another embodiment, the minimal dose volume is about 0.1 mL to about 0.5 mL or any range therein between.

In some embodiments of the methods for treating a microbial (e.g. bacterial and/or fungal) infection, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments of the methods for treating a microbial (e.g. bacterial and/or fungal) infection, the wound area is from about 0.1 cm² to about 250 cm². For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm², about 160 cm², about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides methods for healing a wound in a subject having a diabetic foot infection, comprising administering the subject a therapeutically effective amount of a composition comprising amorphous BisEDT, wherein the composition is a suspension of microparticles comprising said BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, or from about 0.4 μm to about 5 μm, and wherein the composition is applied to the infection (e.g. applied to the surface of the infection) and the wound is healed or substantially healed within 12 weeks of the first administration of the composition. In some embodiments, the BT composition further comprises about 0.05% to about 1.0% Tween 80®, about 0.05 to 40 mM sodium chloride, optionally about 1% to about 10% of methylcellulose (or other polymer such as a carbomer), and optionally about 2 to 20 mM sodium phosphate at about pH. 7.4.

In some embodiments of the methods for healing a wound in a subject having a diabetic foot infection, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration greater than about 100 μg/cm² (e.g. as a dosage from about 250 μg/cm² to about 5,000 μg/cm²). For example, in some embodiments, the bismuth thiol compound in the BT composition is amorphous BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments of the methods for healing a wound in a subject having a diabetic foot infection, the BT composition is administered three times per day, two times per day, once daily, every other day, once every three days, three times per week, once every week, once every other week, once every month, or once every other month. In some embodiments, the wound is healed 4 weeks, 8 weeks or 12 weeks after the first administration of the BT composition. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments of the methods for healing a wound in a subject having a diabetic foot infection, the wound area is from about 0.1 cm² to about 250 cm². For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm, about 160 cm 2, about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides methods for reducing the risk of amputation and/or infection-related surgery in a subject having a diabetic foot infection, comprising administering to the subject a therapeutically effective amount of a composition comprising amorphous BisEDT, wherein the composition is applied to the infection (e.g. applied to the surface of the infection) and the risk of amputation and/or infection-related surgery is reduced from about 1% to about 100% relevant to a similarly situated subject not treated with a therapeutically effective amount of a composition comprising a bismuth-thiol compound. In some embodiments, the composition is a suspension of microparticles comprising said BisEDT wherein at least 70% of the microparticles have a volumetric mean diameter (VMD) from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments, of the methods for reducing the risk of amputation and/or infection-related surgery in a subject having a diabetic foot infection, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration greater than about 100 μg/cm² (e.g. as a dosage from about 250 μg/cm² to about 5,000 μg/cm²). For example, in some embodiments, the bismuth thiol compound in the BT composition is BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm.

In some embodiments, of the methods for reducing the risk of amputation and/or infection-related surgery in a subject having a diabetic foot infection, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments, of the methods for reducing the risk of amputation and/or infection-related surgery in a subject having a diabetic foot infection, the wound area is from about 0.1 cm² to about 250 cm². For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm² about 130 cm² about 135 cm², about 140 cm², about 145 cm², about 150 cm², about 155 cm², about 160 cm², about 165 cm² about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides methods for closing a wound in a subject having a diabetic foot infection, comprising administering to the subject a therapeutically effective amount of a composition comprising amorphous BisEDT. In some embodiments, the composition is a suspension of microparticles comprising said BisEDT wherein at least 70% of the microparticles have a volumetric mean diameter (VMD) from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments, of the methods for closing a wound in a subject having a diabetic foot infection, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration greater than about 100 μg/cm² (e.g. as a dosage from about 250 μg/cm² to about 5,000 μg/cm²). For example, in some embodiments, the bismuth thiol compound in the BT composition is amorphous BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments, of the methods for closing a wound in a subject having a diabetic foot infection, the composition is applied to the infection (e.g. applied to the surface of the infection) and the wound is closed within 12 weeks of the first administration of the composition. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments, of the methods for closing a wound in a subject having a diabetic foot infection, the wound area is from about 0.1 cm² to about 250 cm². For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm², about 160 cm², about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides methods for wound size reduction in a subject having a diabetic foot infection, comprising administering to the subject a therapeutically effective amount of a composition comprising amorphous BisEDT, wherein the composition is applied to the infection (e.g. applied to the surface of the infection) and the wound is reduced in size from about a 1% reduction relative to the original wound size to total elimination of the wound within 12 weeks of the first administration of the composition. In some embodiments, the wound is reduced by about 10%, 20%, 30%, 40%, 50%, 60%, 70%, 80%, or 90%. In some embodiments, the wound is reduced by about 50%.

In some embodiments, of the methods for wound size reduction in a subject having a diabetic foot infection, the composition is a suspension of microparticles comprising said amorphous BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, or from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments, of the methods for wound size reduction in a subject having a diabetic foot infection, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration greater than about 100 μg/cm² (e.g. as a dosage from about 250 μg/cm² to about 5,000 μg/cm²). For example, in some embodiments, amorphous BisEDT is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments, of the methods for wound size reduction in a subject having a diabetic foot infection, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments, of the methods for wound size reduction in a subject having a diabetic foot infection, the wound area is from about 0.1 cm² to about 250 cm². For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 45 cm², about 150 cm², about 155 cm², about 160 cm², about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm². In some embodiments, the wound surface area of said wound is reduced by 50% 12 weeks after the first administration of the BT composition, and the BT composition comprises amorphous BisEDT. In some embodiments, the wound surface area of said wound is reduced by 50% 4 weeks after the first administration of the BisEDT composition. In some embodiments, the wound surface area is measured using digital photographs or hand measurements.

In some embodiments, the present disclosure provides a method for preventing amputation and/or infection-related surgery in a subject having a diabetic foot infection, comprising administering to the subject a therapeutically effective amount of a BT composition. In some embodiments, the BT composition is a suspension of microparticles comprising said amorphous BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments, of the methods for preventing amputation and/or infection-related surgery in a subject having a diabetic foot infection, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration greater than about 100 μg/cm² (e.g. as a dosage from about 250 μg/cm² to about 5,000 μg/cm²). For example, in some embodiments, the bismuth thiol compound in the BT composition is amorphous BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments, the present disclosure provides methods of treating a wound in a subject, wherein the wound is 30 days old or greater, comprising administering to the subject a therapeutically effective amount of a BT composition. In some embodiments, the subject has a diabetic foot infection. In some embodiments, the BT composition is a suspension of microparticles comprising amorphous BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, or from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments, the present disclosure provides methods of treating a wound in a subject, wherein the wound is 30 days old or less, comprising administering to the subject a therapeutically effective amount of a BT composition. In some embodiments, the subject has a diabetic foot infection. In some embodiments, the BT composition is a suspension of microparticles comprising amorphous BisEDT having a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm or from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4. For example, the wound may be about 1 day old, about 2 days old, about 3 days old, about 4 days old, about 5 days old, about 6 days old, about 7 days old, about 8 days old, about 9 days old, about 10 days old, about 11 days old, about 12 days old, about 13 days old, about 14 days old, about 15 days old, about 16 days old, about 17 days old, about 18 days old, about 19 days old, about 20 days old, about 21 days old, about 22 days old, about 23 days old, about 24 days old, about 25 days old, about 26 days old, about 27 days old, about 28 days old, about 29 days old, or about 30 days old.

In some embodiments, of the methods of treating a wound in a subject, wherein the wound is 30 days old or greater, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration greater than about 100 μg/cm² (e.g. as a dosage from about 250 μg/cm² to about 5,000 μg/cm²). For example, in some embodiments, the bismuth thiol compound in the BT composition is amorphous BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments, of the methods of treating a wound in a subject, wherein the wound is 30 days old or less, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration greater than about 100 μg/cm² (e.g. as a dosage from about 250 μg/cm² to about 5,000 μg/cm²). For example, in some embodiments, the bismuth thiol compound in the BT composition is amorphous BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments, of the methods of treating a wound in a subject, wherein the wound is 30 days old or greater, the wound is greater than 2 months old, greater than 3 months old, greater than 4 months old, greater than 5 months old, greater than 6 months old, greater than 7 months old, greater than 8 months old, greater than 9 months old, greater than 10 months old, greater than 11 months old, or greater than 1 year old. In some embodiments, the wound is greater than 2 months old. In some embodiments, the wound is greater than 3 months old.

In any of the embodiments of the methods described herein, the BT composition that is ultimately applied or administered to the subject has a concentration from about 1 μg/mL to about 1,000,000 μg/mL (e.g. about 1 μg/cm² to about 10,000 μg/cm²). In some embodiments, the BT composition has a concentration from about 50 μg/mL to about 100 μg/mL. In some embodiments, the applied BT the BT composition has a concentration from about 250 μg/mL to about 5,000 μg/mL. For example, in some embodiments, the bismuth thiol compound in the BT composition is BisEDT which has a concentration from about 1 μg/mL to about 10,000 μg/mL or from about 50 μg/mL to about 200 μg/mL or from about 250 μg/mL to about 5,000 μg/mL. In some embodiments, the BT composition has a concentration of about 1 μg/mL, about 50 μg/mL, about 100 μg/mL, about 150 μg/mL, about 200 μg/mL, about 250 μg/mL, about 500 μg/mL, about 750 μg/mL, about 1000 μg/mL, about 1500 μg/mL, about 2000 μg/mL, about 2500 μg/mL, about 3000 μg/mL, about 3500 μg/mL, about 4000 μg/mL, about 4500 μg/mL, about 5000 μg/mL, about 5500 μg/mL, about 6000 μg/mL, about 6500 μg/mL, about 7000 μg/mL, about 7500 μg/mL, about 8000 μg/mL, about 8500 μg/mL, about 9000 μg/mL, about 9500 μg/mL, to about 10,000 μg/mL. In some embodiments of the methods described herein, the BT composition that is ultimately applied or administered to the subject has a concentration greater than about 1,000,000 μg/mL.

In a specific embodiment, the present invention may be a pharmaceutical composition comprising bismuth-thiol (BT) composition that comprises amorphous BisEDT suspended therein, wherein the BT composition comprises a plurality of microparticles. In a specific embodiment, the D90 of said microparticles is less than or equal to 4.5 μm, or 4.0 μm, or 3.5 μm, or 3.0 μm, or 2.5 μm, or 2.0 μm, or 1.9 μm, or 1.8 μm, or μm 1.7 μm, or 1.6 μm, or 1.5 μm or any ranges in between. In a specific embodiment, the D90 of said microparticles is less than or equal to 1.9 μm. In another specific embodiment, the D90 of said microparticles is less than or equal to 1.6 μm. In another specific embodiment, the D50 of said microparticles is less than or equal to 2.5 μm, or 2.0 μm, or 1.5 μm, or 1.3 μm, or 1.2 μm, or 1.1 μm, or 1.0 μm, or 0.9 μm, or 0.87 μm, or 0.72 μm or any ranges in between. In another specific embodiment, the D10 of said microparticles is less than or equal to 0.9 μm, or 0.8 μm, or 0.7 μm, or 0.6 μm, or 0.50 μm, or 0.40 μm, or 0.39 μm, or 0.38 μm, or 0.37 μm, or 0.36 μm, or 0.35 μm, or 0.34 μm, or 0.33 μm, or any ranges in between. In a specific embodiment, the pharmaceutical composition comprising bismuth-thiol (BT) composition comprises amorphous BisEDT suspended therein, wherein the BT composition comprises a plurality of microparticles, wherein the D90 of said microparticles is less than or equal to about 1.6 μm. In a specific embodiment, the BT composition comprises about 0.05% to about 1.0% Tween 80®, about 0.05 to 40 mM sodium chloride, optionally about 1% to about 10% of methylcellulose (or polymer such as a carbomer), and optionally about 2 to 20 mM sodium phosphate at about pH. 7. In another specific embodiment, the compositions described above can be administered to a subject for treating a topical wound in a subject, or any specific methods described herein. In another specific embodiment, the compositions described above can be administered to a subject for treating a diabetic foot infection or diabetic foot ulcer. In a specific embodiment, the methods may include administering the compositions described above to a subject, wherein the subject experiences one or more of the following outcomes following the completion of dosing of said composition:
the wound is healed or substantially healed within 12 weeks of the first administration of the composition; and/or
the prevention of amputation and/or infection-related surgery; and/or
the wound is closed partially or fully; and/or
the wound is reduced in size from about a 1% reduction relative to the original wound size to total elimination of the wound; and/or
the wound is 30 days old or greater and is healed or substantially healed.

In some embodiments, the present disclosure provides a method for healing a wound in a subject having a diabetic foot infection, comprising administering the subject a therapeutically effective amount of a composition comprising amorphous BisEDT. In some embodiments, the wound is a diabetic foot ulcer.

In some embodiments, the present disclosure provides a method for healing a wound in a subject having a diabetic foot infection, comprising administering the subject a therapeutically effective amount of a composition comprising amorphous BisEDT, wherein the composition is a suspension of microparticles comprising said BisEDT wherein at least 70% of the microparticles have a volumetric mean diameter (VMD) from about 0.4 μm to about 5 μm, and wherein the composition is applied to the infection and the wound is healed or substantially healed within 12 weeks of the first administration of the composition. In some embodiments, the wound is a diabetic foot ulcer. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4. In some embodiments, the applied BT composition is present on the surface at a concentration from about 1 μg/cm² to about 1,000,000 μg/cm². In some embodiments, the applied BT composition is present on the surface at a concentration from about 50 μg/cm² to about 100 μg/cm². In some embodiments, the applied BT composition is present on the surface at a concentration greater than about 100 μg/cm². In some embodiments, the BT composition is administered three times per day, two times per day, once daily, every other day, once every three days, three times per week, once every week, once every other week, once every month, or once every other month. In some embodiments, the wound is healed 4 weeks, 8 weeks or 12 weeks after the first administration of the BT composition. In some embodiments, the subject is administered multiple doses of the BT composition daily or weekly for a length of time ranging from about one week to about 12 weeks. In some embodiments, the subject is administered multiple doses of the BT composition daily or weekly for a length of about 4 weeks. In some embodiments, the wound area is from about 0.1 cm² to about 250 cm².

In some embodiments, the present disclosure provides a method for wound size reduction in a subject having a diabetic foot infection, comprising administering to the subject a therapeutically effective amount of a composition comprising amorphous BisEDT, wherein the composition is a suspension of microparticles comprising said BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, or from about 0.4 μm to about 5 μm, and wherein the composition is applied to the infection and the wound is reduced in size from about a 1% reduction relative to the original wound size to total elimination of the wound within 12 weeks of the first administration of the composition. In some embodiments, the wound is reduced by about 10⁰/c, 20%, 30%, 40%, 50%, 60%, 70%, 80%, or 90%. In some embodiments, the wound is reduced by at least about 50%. In some embodiments, the wound is a diabetic foot ulcer. In some embodiments, the BT composition further comprises about 0.05% to about 1.0% Tween 80®, about 0.05 to 40 mM sodium chloride, optionally about 1% to about 10⁰/c of methylcellulose, and optionally about 2 to 20 mM sodium phosphate at about pH. 7.4. In some embodiments, the applied BT composition is present on the surface at a concentration from about 1 μg/cm² to about 1,000,000 μg/cm². In some embodiments, the applied BT composition is present on the surface at a concentration from about 50 μg/cm² to about 100 μg/cm². In some embodiments, the applied BT composition is present on the surface at a concentration greater than about 100 μg/cm². In some embodiments, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of about 4 weeks. In some embodiments, the wound area is from about 0.1 cm² to about 250 cm². In some embodiments, the wound surface area of said wound is reduced by at least 50% by 12 weeks after the first administration of the BT composition. In some embodiments, the wound surface area of said wound is reduced by at least 50% by 4 weeks after the first administration of the BisEDT composition. In some embodiments, the wound surface area is measured using digital photographs or hand measurement.

### Embodiments

1. An amorphous form of bismuth-1,2-ethanedithiol (BisEDT).

2. The amorphous form of BisEDT according to embodiment 1, wherein its X-ray powder diffraction pattern does not contain any distinct peaks.

3. The amorphous form of BisEDT according to embodiment 1 or embodiment 2, wherein its X-ray powder diffraction pattern is substantially similar to FIG. 1.

4. The amorphous form of BisEDT according to any one of embodiments 1-3, wherein its differential scanning calorimetry thermogram comprises an exothermic peak at about 168° C.

5. The amorphous form of BisEDT according to embodiment 4, wherein its differential scanning calorimetry thermogram further comprises an endotherm at about 64° C. and/or an endotherm peak at about 112° C. and/or an exotherm peak at about 145° C.

6. The amorphous form of BisEDT according to any of the preceding embodiments, wherein its differential scanning calorimetry thermogram is substantially similar to FIG. 2.

7. The amorphous form of BisEDT according to any of the preceding embodiments having a glass transition at about 101° C.

8. The amorphous form of any one of embodiments 1-7, wherein the amorphous form is at least 90% pure.

9. The amorphous form of embodiment 8, wherein the amorphous form is at least 95% pure.

10. The amorphous form of embodiment 9, wherein the amorphous form is at least 98% pure.

11. A composition comprising an amorphous from of bismuth-1,2-ethanedithiol (BisEDT).

12. The composition of embodiment 11, wherein the composition comprises at least one pharmaceutically acceptable carrier.

13. The composition of embodiment 11, wherein the composition comprises BisEDT in a suspension.

14. A method of treating, managing or lessening the severity of symptoms and infections associated with one or more pulmonary diseases or infections in a subject, the method comprising administering to the subject a bismuth-thiol (BT) composition that comprises amorphous BisEDT according to any one of embodiments 1-10 suspended therein, wherein administering the BT composition is via inhalation, orally or nasally, using an aerosol device.

15. The method of embodiment 14, wherein the method is treating, managing or lessening the severity of cystic fibrosis (CF) symptoms and infections in a subject.

16. A method for healing a wound in a subject having a diabetic foot infection, comprising administering the subject a therapeutically effective amount of a composition comprising amorphous BisEDT according to any one of embodiments 1-10.

17. The method of embodiment 17, wherein the wound is a diabetic foot ulcer.

18. A method of making an amorphous form of BisEDT according to any one of embodiments 1-10, comprising
(a) mixing an acidic aqueous solution that comprises a bismuth salt, with a solvent selected from the group consisting of acetone, acetonitrile, 1,2-dichloroethane, dimethyl sulfoxide, ethyl acetate, isopropanol, methyl tert-butyl ether, and mixtures thereof;
(b) combing the product of (a) with a solution of 1,2-ethanedithiol in a solvent selected from the group consisting of acetone, acetonitrile, 1,2-dichloroethane, dimethyl sulfoxide, ethyl acetate, isopropanol, methyl tert-butyl ether, and mixtures thereof, under conditions and for a time sufficient for formation of a precipitate which comprises the amorphous form of BisEDT.

19. The method of embodiment 18, further comprising recovering the precipitate to remove impurities.

20. The method of embodiment 18 or 19, wherein the bismuth salt is Bi(NO₃)₃.

21. The method of any one of embodiments 18-20, wherein 1,2-ethanedithiol is at a concentration of from about 1% wt/vol to about 20% wt/vol prior to step (b).

22. The method of any one of embodiments 18-21, wherein the acidic aqueous solution is prepared by mixing an aqueous suspension of either Bi (III) sub-nitrate or Bi (III) nitrate pentahydrate with an acid under conditions and for a time sufficient to form a substantially clear solution.

23. The method of embodiment 22, wherein the concentration of either Bi (III) sub-nitrate or Bi (III) nitrate pentahydrate in the aqueous solution is from about 100 mg/mL to about 400 mg/mL.

24. The method of embodiment 22 or embodiment 23, wherein the acid is 70% HNOs.

25. The method of any one of embodiments 22-24, further comprising adding the clear solution to an acidic solution.

26. The method of embodiment 25, wherein the acidic solution is 5% HNO₃.

27. The method of any one of embodiments 18-26, wherein step (b) is performed at a temperature ranging from about 20° C. to about 28° C.

28. A method of treating, managing or lessening the severity of cystic fibrosis (CF) symptoms and infections in a subject, the method comprising administering to the subject a bismuth-thiol (BT) composition that comprises amorphous BisEDT according to any one of embodiments 1-10 suspended therein.

29. The method of embodiment 14-15 or 28, wherein the BT composition comprises a plurality of microparticles wherein at least 70% of said microparticles having a volumetric mean diameter (VMD) from about 0.01 μm to about 2.5 μm.

30. The method of embodiment 14-15 or 28 or embodiment 29, wherein at least 80% of said microparticles having a VMD from about 0.01 μm to about 2.5 μm.

31. The method of any one of embodiments 14-15 or 28-30, wherein at least 90% of said microparticles having a VMD from about 0.01 μm to about 2.5 μm.

32. The method of any one of embodiments 14-15 or 28-31, wherein when the BT composition is aerosolized, at least 70% of the aerosolized liquid droplets have a mass median aerodynamic diameter (MMAD) from about 0.01 μm to about 3 μm.

33. The method of any one of embodiments 14-15 or 28-32, wherein when the BT composition is aerosolized, at least 80% of the aerosolized liquid droplets have a MMAD from about 0.01 μm to about 3 μm.

34. The method of any one of embodiments 14-15 or 28-33, wherein when the BT composition is aerosolized, at least 90% of the aerosolized liquid droplets have a MMAD from about 0.01 μm to about 3 μm.

35. The method of any one of embodiments 14-15 or 28-34, wherein the BT composition comprises BisEDT at a concentration greater than about 0.1 mg/mL, about 0.05% to about 1.0% Tween 80®, about 0.05 to 250 mM sodium chloride, and optionally about 2 to 20 mM sodium phosphate at about pH. 7.4.

36. The method of any one of embodiment 35, wherein range of sodium chloride is about 100 mM to about 200 mM.

37. The method of any one of embodiments 14-15 or 28-36, wherein the subject has at least one pulmonary infection containing one or more bacterial pathogens and/or fungal pathogens.

38. The method of any one of embodiments 14-15 or 28-37, wherein the method comprises at least one of; (i) reducing a bacterial biofilm, (ii) impairing growth of a bacterial biofilm, (iii) preventing initial formation of the bacterial biofilm, and/or (iv) preventing reformation of the bacterial biofilm.

39. The method of any one of embodiments 14-15 or 28-38, wherein the one or more pathogens are selected from Haemophilus influenzae, Pseudomonas aeruginosa, Staphylococcus aureus, Staphylococcus warneri Staphylococcus lugdunensis, Staphylococcus epidermidis, Streptococcus milleri/anginous, Streptococcus pyogenes, non-tuberculosis mycobacteria, Mycobacterium tuberculosis, Burkholderia spp., Achromobacter xylosoxidans, Pandoraea sputorum, Stenotrophomonas maltophilia. Alcaligenes xylosoxidans, Haemophilus pittmaniae, Serratia marcescens, Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, Aspergillus flavus, Morganella morgani, Inquilinus limosus, Ralsionia mannitolilytica, Pandoraea apista, Pandoraea pnomenusa, Pandoraea sputorum, Bdellovibrio bacterioworus, Bordetella bronchiseptica, Vampirovibrio chlorellavorus, Actinobacter baumanni, Cupriadidus metallidurans, Cupriavidus pauculus, Cupriavidus respiraculi, Delftia acidivordans, Erophilia dermatitidis, Herbas pirillum frisingense, Herbaspirillum seropedicae, Klebsiella pneumoniae, Pandoraea norimbergensis, Pandoraea pulmonicola, Pseudomonas mendocina, Pseudomonas pseudoalcaligenes, Pseudomonas putida, Pseudomonas stutzeri, Ralsionia insidiosa, Ralstonia pickettii, Neisseria gonorrhoeae, NDM-1 positive E. coli, Enterobacter cloaca, Vancomycin-resistant E. faecium, Vancomycin-resistant E. faecalis, E. faecium, E. faecalis, Clindamycin-resistant S. agalactiae, S. agalactiae, Bacteroides fragilis, Clostridium difficile, Streptococcus pneumonia, Moraxella catarrhalis, Haemophilus haemolyticus, Haemophilus parainfluenzae, Chlamydophilia pneumoniae, Mycoplasma pneumoniae, Atopobium, Sphingomonas, Saccharibacteria, Leptotrichia, Capnocytophaga, Oribacterium, Aquabacterium, Lachnoanaerobaculum, Campylobacter, Acinetobacter; Agrobacterium; Bordetella; Brevundimonas; Chryseobacterium; Delftia; Enterobacter; Klebsiella; Pandoraea; Pseudomonas; Ralstonia, and Prevotella.

40. The method of any one of embodiments 14-15 or 28-29, wherein the one or more pathogens are non-tuberculosis mycobacteria.

41. An aerosol comprising a plurality of dispersed liquid droplets in a gas, said liquid droplets comprising a BT composition comprising amorphous BisEDT according to any one of embodiments 1-10 suspended therein; and
wherein at least 70% of the liquid droplets have a MMAD from about 0.9 μm to about 3 μm.

42. The aerosol of embodiment 41, wherein prior to aerosolization, the BT composition comprises a plurality of microparticles wherein at least 70% of said microparticles have a VMD from about 0.01 μm to about 2.5 μm.

43. The aerosol of embodiment 41 or embodiment 42, wherein least 80% of the liquid droplets have a MMAD from about 003 μm to about 3 μm.

44. The aerosol of any one of embodiments 41-43, wherein least 90% of the liquid droplets have a MMAD from about 0.03 μm to about 3 μm.

45. The aerosol of any one of embodiments 41-44, wherein prior to aerosolization, the BT composition comprises a plurality of microparticles wherein at least 80% of said microparticles have a VMD from about 0.01 μm to about 2.5 μm.

46. The aerosol of any one of embodiments 41-45, wherein prior to aerosolization, the BT composition comprises a plurality of microparticles wherein at least 90% of said microparticles have a VMD from about 0.01 μm to about 2.5 μm.

47. The aerosol of any one of embodiments 41-46, wherein the droplets further comprise Tween 80 and optionally a buffer at a pH of about 7.4; and/or sodium chloride.

48. The aerosol of any one of embodiments 41-47, wherein a substantial amount of the BisEDT compounds are deposited to the deep lung region.

49. The aerosol of any one of embodiments 41-48, wherein at least 80% of the BisEDT compounds are deposited to the deep lung region.

50. A pharmaceutical composition comprising bismuth-thiol (BT) composition that comprises amorphous BisEDT according to embodiments 1-10 suspended therein, wherein the BT composition comprises a plurality of microparticles, wherein the D90 of said microparticles is less than or equal to 1.9 μm.

51. The pharmaceutical composition of embodiment 50, comprising bismuth-thiol (BT) composition comprises BisEDT suspended therein, wherein the BT composition comprises a plurality of microparticles, wherein the D90 of said microparticles is less than or equal to about 1.6 μm.

52. The pharmaceutical composition of embodiments 50 or 51, wherein at least 70% of said microparticles having a volumetric mean diameter from about 0.01 μm to about 2.5 μm.

53. The pharmaceutical composition according to any of embodiments 50-52, wherein at least 90% of said microparticles having a volumetric mean diameter from about 0.6 μm to about 2.5 μm.

54. A method of treating, managing or lessening the severity of symptoms and infections associated with one or more pulmonary diseases or infections in a subject, the method comprising administering to the subject a bismuth-thiol (BT) composition that comprises amorphous BisEDT according to nay of embodiments 1-10, wherein the BT composition comprises a plurality of microparticles wherein at least 70% of said microparticles having a volumetric mean diameter from about 0.01 μm to about 2.5 μm, and wherein when the BT composition is aerosolized, at least 70% of the aerosolized liquid droplets have a MMAD from about 0.03 μm to about 3 μm.

55. The method of embodiment 54, wherein the one or more pulmonary diseases or infections are not the result of or associated with cystic fibrosis.

56. The method of embodiment 54 or 55, wherein the pulmonary infection is bronchiectasis infection, pneumonia, valley fever, allergic bronchopulmonary aspergillosis (ABPA), ventilator acquired pneumonia, hospital acquired pneumonia, community acquired pneumonia, ventilator associated tracheobronchitis, lower respiratory tract infection, non-tuberculous Mycobacteria (NTM), anthrax, legionellosis, pertussis, bronchitis, Bronchiolitis, COPD-associated infection, and post-lung transplantation.

57. The method of embodiment 56, wherein the pulmonary infection is non-tuberculous Mycobacteria (NTM).

58. A method for healing a wound in a subject having a diabetic foot infection, comprising administering the subject a therapeutically effective amount of a composition comprising amorphous BisEDT according to any one of embodiments 1-10, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is healed or substantially healed within 12 weeks of the first administration of the composition.

59. The method of embodiment 58, wherein the wound is a diabetic foot ulcer.

60. The method of embodiment 16-17 or 58 or 59, wherein the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

61. The method of any one of embodiments 16-17 or 58-60, wherein the applied BT composition is present on the surface at a concentration from about 1 μg/cm² to about 1,000,000 μg/cm².

62. The method of any one of embodiments 16-17 or 58-61, wherein the applied BT composition is present on the surface at a concentration from about 50 μg/cm² to about 100 μg/cm².

63. The method of any one of embodiments 16-17 or 58-62, wherein the applied BT composition is present on the surface at a concentration greater than about 100 μg/cm².

64. The method of any one of embodiments 16-17 or 58-63, wherein the BT composition is administered three times per day, two times per day, once daily, every other day, once every three days, three times per week, once every week, once every other week, once every month, or once every other month.

65. The method of any one of embodiments 16-17 or 58-64, wherein the wound is healed 4 weeks, 8 weeks or 12 weeks after the first administration of the BT composition.

66. The method of any one of embodiments 16-17 or 58-65, wherein the subject is administered multiple doses of the BT composition daily or weekly for a length of time ranging from about one week to about 12 weeks.

67. The method of any one of embodiments 16-17 or 58-66, wherein the subject is administered multiple doses of the BT composition daily or weekly for a length of about 4 weeks.

68. The method of any one of embodiments 16-17 or 58-67, wherein the wound area is from about 0.1 cm² to about 250 cm².

69. A method for wound size reduction in a subject having a diabetic foot infection, comprising administering to the subject a therapeutically effective amount of a composition comprising amorphous BisEDT according to embodiments 1-10, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is reduced in size from about a 1% reduction relative to the original wound size to total elimination of the wound within 12 weeks of the first administration of the composition.

70. The method of any one of embodiments 16-17 or 69, wherein the wound is reduced by about 10%, 20%, 30%, 40%, 50%, 60%, 70%, 80%, or 90%.

71. The method of embodiment 16-17 or 69 or 70, wherein the wound is reduced by at least about 50%.

72. The method of any one of embodiments 16-17 or 69-71, wherein the wound is a diabetic foot ulcer.

73. The method of any one of embodiments 16-17 or 69-72, wherein the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

74. The method of any one of embodiments 16-17 or 69-73, wherein the applied BT composition is present on the surface at a concentration from about 1 μg/cm² to about 1,000,000 μg/cm².

75. The method of any one of embodiments 16-17 or 69-74, wherein the applied BT composition is present on the surface at a concentration from about 50 μg/cm² to about 100 μg/cm².

76. The method of any one of embodiments 16-17 or 69-75, wherein the applied BT composition is present on the surface at a concentration greater than about 100 μg/cm².

77. The method of any one of embodiments 16-17 or 69-76, wherein the BT composition is administered three times per day, two times per day, once daily, every other day, once every three days, three times per week, once every week, once every other week, once every month, or once every other month.

78. The method of any one of embodiments 16-17 or 69-77 wherein the BT composition is administered once daily or three times per week.

79. The method of any one of embodiments 16-17 or 69-78, wherein the subject is administered multiple doses of the BT composition daily or weekly for a length of time ranging from about one week to about 12 weeks.

80. The method of any one of embodiments 16-17 or 69-79, wherein the subject is administered multiple doses of the BT composition daily or weekly for a length of about 4 weeks.

81. The method of any one of embodiments 16-17 or 69-80, wherein the wound area is from about 0.1 cm² to about 250 cm².

82. The method of any one of embodiments 16-17 or 69-81, wherein the wound surface area of said wound is reduced by at least 50% by 12 weeks after the first administration of the BT composition.

83. The method of any one of embodiments 16-17 or 69-82, wherein the wound surface area of said wound is reduced by at least 50% by 4 weeks after the first administration of the BisEDT composition.

84. The method of any one of embodiments 16-17 or 69-83, wherein the wound surface area is measured using digital photographs or hand measurement.

85. A pharmaceutical composition comprising bismuth-thiol (BT) composition that comprises amorphous BisEDT according to any one of embodiments 1-10 suspended therein, wherein the BT composition comprises a plurality of microparticles, wherein the D90 of said microparticles is less than or equal to 1.9 μm.

86. The pharmaceutical composition of embodiment 85, wherein the BT composition comprises a plurality of microparticles, wherein the D90 of said microparticles is less than or equal to about 1.6 μm.

87. A method for healing a wound in a subject having a diabetic foot infection, comprising administering the subject a therapeutically effective amount of a composition of embodiment 85 or embodiment 86.

### EXAMPLES

The following examples are provided to illustrate the present disclosure, and should not be construed as limiting thereof. Additional experimental procedures and details can be found in International Patent Application Nos. PCT/US2010/023108, PCT/US2011/023549, and PCT/US2011/047490, PCT/US2019/044489, and PCT/US2019/044495 which are hereby incorporated by reference in their entireties for all purposes.

### Example 1: Amorphous BisEDT Characterization

Amorphous BisEDT was characterized by XRPD and DSC. Modulated and temperature cycling DSC analyses were also conducted to determine the glass transition temperature and investigate any potential form change/recrystallization events upon heating.

By XRPD, the pattern contains diffuse scattering without any sharp peaks (FIG. 1).

The conventional DSC for the amorphous BisEDT shows multiple events and displays broad, small endotherms at 64° C. and 112° C. (FIG. 2). These are potentially associated with volatile release and relaxation enthalpy due to a glass transition, respectively. The thermogram also shows a small exotherm at 145° C. (peak max) and a very sharp exotherm at 168° C. (peak max).

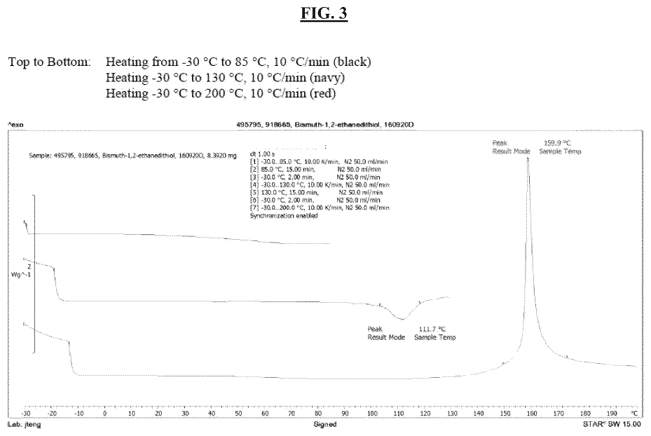

For temperature cycling DSC the material was heated to the temperature past the first broad endotherm to remove any associated volatiles (85° C.) and was quenched to sub-ambient temperature. The sample was then heated to 130° C., above the temperature of the second small endotherm, quenched to sub-ambient temperature, and finally re-heated to 200° C. Based on the data, no apparent glass transition is observed which could be due to crystallization of the amorphous solids upon quenching. The strong exotherm at 160° C. (peak max) is possibly due to degradation, based on hotstage microscopy of the crystalline lot (FIG. 3).

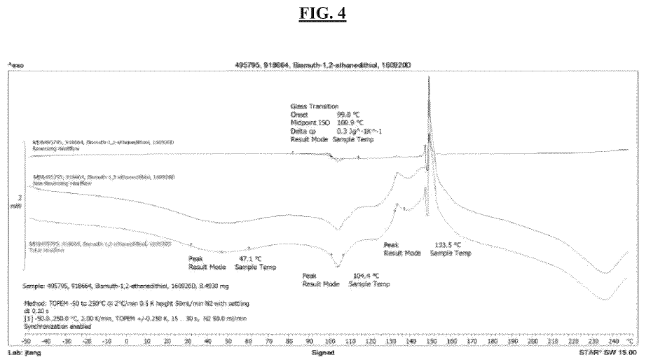

In an attempt to further investigate the glass transition temperature, modulated DSC was conducted with lot 160920D (FIG. 4). A step change on the reversing heat flow curve is observed with a midpoint at 101° C. and is typical of the glass transition. An endotherm seen at 104° C. (peak max) on the total heat flow thermogram is likely associated with the relaxation enthalpy of the glass transition event. The small exotherm at 134° C. (peak max) could be due to crystallization; however, this is immediately followed by heat flow fluctuations which are attributable to decomposition. Thus, the amorphous material exhibited a glass transition at about 101° C.

TABLE 1
Physical Characterization of Amorphous BisEDT

| | Analytical | |
|---|---|---|
| Compound | Technique | Results |
| X-ray amorphous | XRPD | Diffuse scatter |
| BisEDT | DSC | Broad endotherm at 64° C. (peak max) |
| | | Small endotherm at 112° C. (peak max) |
| | | Small exotherm at 145° C. (peak max) |
| | | Sharp strong exotherm at 168° C. (peak max) |
| | Temperature | Cycle 1: Heat to 85° C./min |
| | cycling DSC | Broad, weak endotherm at 75° C. (approximate peak max) |
| | | Cycle 2: Hold, 15 min |
| | | Cycle 3: Quench cool to −30° C., hold, 2 min |
| | | Cycle 4: Heat to 130° C., 10° C./min |
| | | Small endotherm at 112° C. (peak max) |
| | | Cycle 5: Hold, 15 min |
| | | Cycle 6: Quench cool to −30° C., hold, 2 min |
| | | Cycle 7: Heat to 200° C., 10° C./min |
| | | Sharp exotherm at 160° C. (peak max) |
| | Modulated | Reversing heat flow |
| | DSC | T_{g} at 101° C. (midpoint of step change) (ΔCₚ 0.3 J/(g*K) |
| | | Non-reversing and total heat flow |
| | | Broad weak endotherm at 47° C. (peak max), |
| | | Small endotherm at 104° C. (peak max) |
| | | Small exotherm at 134° C. (peak max) |
| | | Heat flow fluctuations above 140° C. |

### Example 3: Synthesis of Amorphous BisEDT

General Procedure using Bismuth (III) Sub-Nitrate: Solids of Bi (III) sub-nitrate were suspended in H₂O (initial solid loading was ˜210-230 mg/mL). 70% HNO₃ was then added until a clear solution was obtained. This solution was added to a 5% aqueous solution of HNO₃, followed by addition of the specified solvent. In a separate vessel, a solution of 1,2-ethanedithiol was prepared in the specified solvent (at a concentration of ˜5% or 13%) and added in portions to the main reactor while stirring. The resulting solids were slurried at ambient temperature for a given duration. The suspension was then allowed to settle and the majority of solvent decanted and replaced with the same solvent. After the suspension was slurried at ambient temperature for a given amount of time, solids were isolated via vacuum filtration and either collected/analyzed or the cycle was repeated.

General Procedure using Bismuth (III) Nitrate Pentahydrate: Solids of Bi (III) nitrate pentahydrate were suspended in H₂O (initial solid loading was ˜230 mg/mL). 70% HNO₃ was then added until a clear solution was obtained. This solution was added to a 5% aqueous solution of HNO₃ followed by addition of the specified solvent. In a separate vessel, a solution of 1,2-ethanedithiol was prepared in the specified solvent (at a concentration of 5%) and added in portions to the main reactor while stirring. The resulting solids were slurried at ambient temperature for a given duration. The suspension was then allowed to settle and the majority of solvent decanted and replaced with the same solvent. After the suspension was slurried at ambient temperature for a given amount of time, solids were isolated via vacuum filtration and either collected/analyzed or the cycle was repeated.

Solvent Studies: International Patent Application Nos. PCT/US2010/023108, PCT/US2011/023549, PCT/US2011/047490 described a synthesis of BisEDT under ethanolic solvent conditions, the product of which was believed to be amorphous; however, unexpectedly this form of BisEDT was discovered to be crystalline. The inventors of the present invention discovered that solvent selection played a critical role in the formation of crystalline and amorphous forms of BisEDT. While ethanol and a variety of other common solvents produced crystalline forms of BisEDT, acetone, acetonitrile, 1,2-dichloroethane, dimethyl sulfoxide, ethyl acetate, isopropanol, and methyl tert-butyl ether were discovered to produce amorphous BisEDT.

TABLE 2
Effect of solvent on the synthesis of amorphous BisEDT From Bi (III) Sub-Nitrate

| | | Initial Solid | 1,2-Ethanedithiol | | |
|---|---|---|---|---|---|
| Scale | Solvent (b) | Loading | molar equiv. | Work-up Conditions | XRPD Results |
| 256 mg | Acetone (4 mL) | 213 mg/mL | 4.5 eq. total (1.5 eq. | Slurry, RT, 1 day. Re-suspended | X-ray amorphous + |
| | | | initially added as a ~5% | solids in Acetone; slurry, RT, 1 | peaks of Form A |
| | | | solution in Acetone, | day. Isolated solids via VF, | |
| | | | 3 eq. added | washed filtrate with Acetone. | |
| | | | neat after 1 day | Re-suspended solids in Acetone; | |
| | | | stirring) | slurry, RT, 4 days. Isolated via | |
| | | | | VF. | |
| 509 mg | Acetone (9 mL) | 232 mg/mL | 3 eq. total | Slurry, RT, 1 day. Re-suspended | X-ray amorphous + |
| | | | (1.5 eq. initially | solids in Acetone; slurry, RT, 4 | peaks of Form A |
| | | | added as a ~5% | days. Isolated solids via VF. | |
| | | | solution in Acetone, | | |
| | | | 1.5 eq. added neat | | |
| | | | after 1 day stirring) | | |
| 253 mg | CAN (4 mL) | 211 mg/mL | 4.5 eq. total (1.5 eq. | Slurry, RT, 1 day. Re-suspended | X-ray amorphous |
| | | | initially added as a ~5% | solids in CAN; slurry, RT, 1 day. | |
| | | | solution in ACN, | Isolated solids via VF, washed | |
| | | | 3 eq. added | filtrate with CAN. Re-suspended | |
| | | | neat after 1 day | solids in CAN; sluny, RT, 4 | |
| | | | stirring) | days. Isolated via VF. | |
| 499 mg | CAN (9 mL) | 227 mg/mL | 3 eq. total (1.5 eq. | Slurry, RT, 1 day. Re-suspended | Insufficient |
| | | | initially added as a ~5% | solids in CAN; slurry, RT, 4 | amount of solids |
| | | | solution in ACN, | days. Isolated solids via VF. | for analysis |
| | | | 1.5 eq. added | | |
| | | | after 1 day stirring) | | |
| 503 mg | DCE (9 mL) | 229 mg/mL | 4.5 eq. (added as a ~5% | Slurry, RT, 5 days. | X-ray amorphous |
| | | | solution in DCE) | Re-suspended solids in DCE; | |
| | | | | sluny, RT, 1 day. Isolated solids | |
| | | | | via VF, washed DCE. Re- | |
| | | | | suspended solids in DCE; slurry, | |
| | | | | RT, 1 day. Isolated via VF. | |
| 505 mg | Dioxane (9 mL) | 230 mg/mL | 4.5 eq. (added as a ~13% | Sluny, RT, 1 day. Re-suspended | Material B |
| | | | solution in Dioxane) | solids in Dioxane; slurry, RT, 4 | |
| | | | | days (pale yellow solids). | |
| | | | | Isolated solids via VF. | |
| 508 mg | DMF (9 mL) | 231 mg/mL | 4.5 eq. (added as a ~13% | Sluny, RT, 1 day. Re-suspended | Form A |
| | | | solution in DMF) | solids in DME; slurry, RT, 4 | |
| | | | | days. Isolated solids via VF. | |
| 499 mg | DMSO (9 mL.) | 227 mg/mL | 4.5 eq.(added as a ~5% | Sluny, RT, 5 days. Attempted to | X-ray amorphous |
| | | | solution in DMSO) | re-suspend solids in DMSO; | |
| | | | | obtained clear solution. Added | |
| | | | | H2O (yellow solids precipitated); | |
| | | | | slurry, RT, 2 days. Isolated | |
| | | | | solids via VF, washed with H₂O. | |
| 502 mg | EtOAc (9 mL) | 228 mg/mL | 4.5 eq.(added as a ~13% | Slurry, RT, 1 day | X-ray amorphous |
| | | | solution in EtOAc) | (flocculent solids clumped | |
| | | | | together). Re-suspended solids in | |
| | | | | EIOAc; slurry, RT, 4 days. | |
| | | | | Isolated solids via VF. | |
| 260 mg | EtOH (4 mL) | 217 mg/mL | 4.5 eg. total (1.5 eq. | Sluny, RT, 1 day. Re-suspended | Form A, |
| | | | initially added as a ~5% | solids in EtOH; slurry, RT, 1 | disordered |
| | | | solution in EtOH, | day. Isolated solids via VF, | |
| | | | 3 eq. added | washed filtrate with EtOH. Re- | |
| | | | neat after 1 day | suspended solids in EtOH; | |
| | | | stirring) | slurry, RT, 4 days. Isolated via | |
| | | | | VF. | |
| 505 mg | EtOH (9 mL) | 230 mg/mL | 3 eq. total (1.5 eq. | Slurry, RT, I day. Re-suspended | Form A, halos |
| | | | initially added as a ~5% | solids in EtOH; slurry, RT, 4 | present |
| | | | solution in EtOH, | days. Isolated solids via VF. | |
| | | | 1.5 eq. added | | |
| | | | neat after 1 day | | |
| | | | stirring) | | |
| 502 mg | EtOH/H₂O | 228 mg/mL | 4.5 eq. (added as a ~5% | Slurry, RT, 4 days | Form A + Material |
| | 50/50 (c) | | solution in EtOH) | Re-suspended solids in 50/50 | F |
| | | | | EtOH/H₂O; slurry, RT, 3 days. | |
| | | | | Isolated solids via VF. | |
| 508 mg | H₂O | 231 mg/mL | 3 eq. total (1.5 eq. | Slurry, RT, 1 day. Re-suspend | Form A |
| | | | initially added neat, | solids in H₂O; slurry, RT, 4 days. | |
| | | | 1.5 cq. added neat | Isolated solids via VF. | |
| | | | after 1 day stirring) | | |
| 254 mg | IPA (4 mL) | 212 mg/mL | 4.5 eq. total (1.5 eq. | Slurry, RT, 1 day. Re-suspend | X-ray amorphous |
| | | | initially added as a ~5% | solids in IPA; slurry, RT, 1 day. | |
| | | | solution in IPA, | Isolated solids via VF, washed | |
| | | | 3 eq. added neat after | solids with IPA. Re-suspended | |
| | | | 1 day stirring) | solids in IPA; slurry, RT, 4 days. | |
| | | | | Isolated solids via VF. | |
| 506 mg | IPA (9 mL) | 230 mg/mL | 3 eq. total (1.5 eq. | Slurry, RT, 1 day. Re-suspend | X-ray amorphous |
| | | | initially added neat, ~5% | solids in IPA; slurry, RT, 4 days. | |
| | | | solution in IPA, | Isolated solids via VF. | |
| | | | 1.5 eq. added neat | | |
| | | | after 1 day stirring) | | |
| 509 mg | MeOH (9 mL) | 231 mg/mL | 4.5 eq. added as a ~13% | Slurry, RT, 1 day. Re-suspended | Form A |
| | | | solution in MeOH) | solids in MeOH; slurry, RT, 4 | |
| | | | | days. Isolated via VF. | |
| 505 mg | MTBE | 230 mg/mL | 4.5 eq. added as a ~5% | Sluny, RT, 4 days. | X-ray amorphous |
| | | | solution in MTBE) | Re-suspended solids in MTBE; | |
| | | | | slurry, RT, 3 days. Isolated | |
| | | | | solids via VF. | |
| 495 mg | THF | 225 mg/mL | 4.5 eq. added as a ~5% | Slurry, RT, 5 days. | Material B |
| | (9 mL) (d) | | solution in THE) | Re-suspended solids in THF; | |
| | | | | sluny, RT, 1 day. Isolated solids | |
| | | | | via VF, washed with THF (solids | |
| | | | | had a pale yellow, almost white, | |
| | | | | color). Re-suspended white | |
| | | | | solids in THE; slurry, RT, 1 day. | |
| | | | | Isolated via VF. | |

(a) Amounts, temperature, and duration of experiments are approximate.
(b) Specified amount does not include the solvent used to prepare the 1,2-ethanedithiol solution.
(c) 23.5 mL EtOH added, does not include EtOH used to prepare the 1,2-ethanedithiol solution; 50/50 ratio includes total amount of solvents used throughout experiment.
(d) Non-stabilized THF.

TABLE 3
Effect of solvent on the synthesis of amorphous BisEDT From Bi (III) nitrate pentahydrate

| | | Initial Solid | 1,2-Ethanedithiol molar | | |
|---|---|---|---|---|---|
| Scale | Solvent (b) | Loading | equiv. | Work-up Conditions | XRPD Results |
| 503 mg | Acetone (9 mL) | 229 mg/mL | 1.5 eq. (added as a ~5% | Slurry, RT, 2 days. Re-suspended | Insufficient |
| | | | solution in Acetone) | solids in Acetone; slurry, RT, 4 | amount for |
| | | | | days. Isolated solids via VF, washed | analysis |
| | | | | several times with Acetone. | |
| 508 mg | | 231 mg/mL | 1.5 eq. | Slurry, RT, 2 days. Re-suspended | Form A, slight |
| | | | (added as a ~5% solution | solids in Acetone; slurry, RT, 4 | increase in |
| | | | in Acetone) | days. Isolated solids via VF and re- | crystallinity |
| | | | | suspended in Acetone; slurry, RT, 1 | |
| | | | | day. Isolated solids via VF. | |
| 497 mg | ACN (9 mL) | 226 mg/mL | 1.5 eq. (added as a ~5% | Slurry, RT, 2 days. Re-suspended | Form A |
| | | | solution in ACN) | solids in ACN; slurry, RT, 4 days. | |
| | | | | Isolated solids via VF; washed | |
| | | | | several times with ACN. | |
| 502 mg | DCE (9 mL) | 228 mg/mL | 1.5 eq. (added as a ~5% | Slurry, RT, 5 days. Re-suspended | X-ray amorphous |
| | | | solution in DCE) | solids in DCE; slutry, RT, 7 days. | |
| | | | | Isolated solids via VF and re- | |
| | | | | suspended in DCE; slurry, RT, 2 | |
| | | | | days. Isolated solids via VF. | |
| 508 mg | Dioxane (9 mL) | 231 mg/mL | 1.5 eq. (added as a ~5% | Sluny, RT, 5 days. Re-suspended | Form A |
| | | | solution in Dioxane) | solids in dioxane; sluny, RT, 7 | |
| | | | | days. Isolated solids via VF and re- | |
| | | | | suspended in dioxane; slurry, RT, 2 | |
| | | | | days. Isolated solids via VF. | |
| 499 mg | DMF (9 mL) | 227 mg/mL | 1.5 eq. (added as a ~5% | Slurry, RT, 2 days. Re-suspended | Form A |
| | | | solution in DMF) | solids in DMF; slurry, RT, 4 days. | |
| | | | | Isolated solids via VF and re- | |
| | | | | suspended in DMF; slurry, RT, 1 | |
| | | | | day. Isolated solids via VF. | |
| 504 mg | DMSO (9 mL) | 229 mg/mL | 1.5 eq (added as a ~5% | Slurry, RT, 5 days. Re-suspended | Form A |
| | | | solution in DMSO) | solids in DMSO/H₂O; slurry, RT, 7 | |
| | | | | days. Isolated solids via VF and re- | |
| | | | | suspended in H₂O and DMSO (~2:1 | |
| | | | | H₂O:DMSO). Slurry, RT, 2 days. | |
| 504 mg | EtOAc (9 mL) | 229 mg/mL | 1.5 eq. (added as a ~5% | Slurry, RT, 2 days. Re-suspended | Form A |
| | | | solution in EtOAc) | solids in EtOAc; slurry, RT, 4 days. | |
| | | | | Isolated solids via VF; washed | |
| | | | | several times with EtOAc. | |
| 509 mg | EIOH (9 mL) | 231 mg/mL | 1.5 eq. (added as a ~5% | Slurry, RT, 2 days. Re-suspended | Form A |
| | | | solution in EtOH) | solids in EtOH; slurry, RT, 4 days. | |
| | | | | Isolated solids via VF; washed | |
| | | | | several times with EtOH. | |
| 503 mg | EtOH/H₂O | 229 mg/mL | 1.5 eq. (added as a ~5% | Slurry, RT, 4 days. Re-suspended | Form A |
| | 50/50 (c) | | solution in EtOHI) | solids in 50/50 EtOH/H₂O; slurry, | |
| | | | | RT, 3 days. Isolated solids via VF. | |
| 494 mg | H₂O | 225 mg/mL | 1.5 eq. (added neat) | Slurry, RT, 2 days. Re-suspended | Form A |
| | | | | solids in H₂O; slurry, RT, 4 days. | |
| | | | | Isolated solids via VF and re- | |
| | | | | suspended in H₂O; slurry, RT, 1 | |
| | | | | day. Isolated solids via VF. | |
| 512 mg | IPA (9 mL) | 233 mg/mL | 1.5 eq. (added as a ~5% | Sluny, RT, 2 days. Re-suspended | Form A |
| | | | solution in IPA) | solids in IPA; slurry, RT, 4 days. | disordered |
| | | | | Isolated solids via VF and re- | |
| | | | | suspended in IPA; slurry, RT, 1 day. | |
| | | | | Isolated solids via VF. | |
| 512 mg | MeOH (9 mL) | 233 mg/mL | 1.5 eq. (added as a ~5% | Slurry, RT, 2 days. Re-suspended | Form A |
| | | | solution in MeOH) | solids in MeOH; slurry, RT, 4 days. | |
| | | | | Isolated solids via VF; washed | |
| | | | | several times with MeOH. | |
| 498 mg | MTBE (9 mL) | 226 mg/mL | 1.5 eq. (added as a ~5% | Sluny, RT, 4 days. Re-suspended | Material G |
| | | | solution in MTBE) | solids in MTBE; slurry, RT, 3 days. | |
| | | | | Isolated solids via VF. | |
| 509 mg | THF (9 mL) (d) | 231 mg/mL | 1.5 eq. (added as a ~5% | Sluny, RT, 5 days (white/pale | Material B |
| | | | solution in THE) | yellow solids). Re-suspended solids | |
| | | | | in THF; slurry, RT, 7 days. Isolated | |
| | | | | solids via VF and re-suspended in | |
| | | | | THF; slurry, RT, 2 days. Isolated | |
| | | | | solids via VF. | |

(a) Amounts, temperature, and duration of experiments are approximate.
(b) Specified amount does not include the solvent used to prepare the 1,2-ethanedithiol solution.
(c) 23.5 mL EtOH added, does not include EtOH used to prepare the 1,2-ethanedithiol solution; 50/50 ratio includes total amount of solvents used throughout experiment.
(d) Non-stabilized THF.

### Example 3: Instrumental Techniques

### X-ray Powder Diffraction (XRPD): Transmission Geometry

XRPD patterns were collected with a PANalytical X'Pert PRO MPD diffractometer Using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry.

A beam-stop, short antiscatter extension, and an antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b.

XRPD patterns were collected with a PANalytical Empyrean diffractometer using an incident beam of CU radiation produced using a long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X'Celerator) located 240 mm from the specimen and Data Collector software v. 5.5.

XRPD: Reflection Geometry. XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a/thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 2.2b.

Differential Scanning Calorimetry (DSC). DSC was performed using a Mettler-Toledo DSC3+ differential scanning calorimeter. A tau lag adjustment was performed with indium, tin, and zinc. The temperature and enthalpy were adjusted with octane, phenyl salicylate, indium, tin and zinc. The adjustment was then verified with octane, phenyl salicylate, indium, tin, and zinc. The sample was placed into a hermetically sealed aluminum DSC pan, and the weight was accurately recorded. The pan lid was pierced and then inserted into the DSC cell. A weighed aluminum pan configured as the sample pan was placed on the reference side of the cell. The pan lid was pierced prior to sample analysis. The data acquisition parameters for the thermogram are displayed in the image in the Physical Characterization Data section of this report.

Temperature Modulated Differential Scanning Calorimetry (modulated DSC): Temperature Modulated DSC (TMDSC) was performed using Mettler-Toledo DSC3+ differential scanning calorimeter. TOPEM® overlays the isothermal or ramped temperature with a time series of random temperature pulses of different durations. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan lid was pierced and the pan was then inserted into the DSC cell. A weighed aluminum pan configured as the sample pan was placed on the reference side of the cell. The pan lid was pierced prior to sample analysis. The data was collected from −50° C. to 250° C. at 2° C./min with a modulation amplitude of =0.25 K and a 15 to 30 second period with an underlying heating rate of 2.0 K/minute. The reported glass transition temperature is obtained from the inflection point of the step change in the reversing heat flow versus temperature curve.

### Example 4: Studies on Processing Conditions on BisEDT Particle Size Distribution

It was observed that careful control of the reaction temperature and the rate of 1,2 ethanedithiol addition had pronounced impact on crystalline BisEDT particle size distribution. Representative syntheses are shown below for BisEDT synthesized at 20° C. with a 1.25 hour addition of 1,2-ethane via syringe pump and BisEDt synthesized at 15° C. with a 1 hour addition of 1,2-ethane via syringe pump. Table 15 below shows that temperature conditions play a critical role in particle size distribution, with processing temperatures in the range of 20-30° C. providing BisEDT particles that are both small and uniform in particle size (such as a D90 below 2 microns).

Representative synthesis of BisEDT at 20° C. with 1.25 hour addition of thiol via syringe pump, and polypropylene cloth for filtration: BisEDT synthesis was performed on 10-g scale. To a 1-L jacketed reactor was charged USP water (480 mL, 48 vol), followed by 70% HNO₃ (34 mL, 3.4 vol). A solution of bismuth subnitrate (10 g, 6.84 mmols) in water (43 mL, 4.3 vol) and 70% HNO₃ (14 mL, 1.4 vol) was added at 20° C. The reaction mixture was cooled to 15° C. for addition of 95% Ethanol. The 95% ethanol (180 mL, 18 vol) was then added slowly. (Ethanol addition is exothermic, temperature reached 22° C.). The temperature was then adjusted back to 20° C. This was followed by dropwise addition of 1,2 ethanedithiol (4.3 mL, 7.5 mmols in 95% ethanol in 94 mL, 9.4 vol) over a period of 1.25 hour with the batch temperature at 20° C. during which time it turned into a yellow suspension. The reaction was stirred at 20° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.81 g 91.0%).

Representative synthesis of BisEDT at 15° C. with 1 hour addition of thiol via syringe pump, and polypropylene cloth for filtration: The synthesis BisEDT was performed on 10-g scale, temperature profile was studied with data logger. Ethane dithiol was added at 15° C. over 1 hour via syringe pump and the filtration was performed using PP filter cloth. To a 1-L jacketed reactor was charged USP water (480 mL, 48 vol) and cooled to 15° C., followed by 70% HNO₃ (34 mL, 3.4 vol). A solution of bismuth subnitrate (10 g, 6.84 mmols) in water (43 mL, 4.3 vol) and 70% HNO₃ (14 mL, 1.4 vol) was added at the same temperature. The 95% ethanol (180 mL, 18 vol) was then added slowly. (Ethanol addition is exothermic, temperature reached 22.5° C.). It was then allowed to cool to 15° C. This was followed by dropwise addition of 1,2 ethanedithiol (4.3 mL, 7.5 mmols in 95% ethanol in 94 mL, 9.4 vol) over an hour with the batch temperature at 15° C. The reaction was allowed to stir at 15° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.43 g 87.8%).

TABLE 4
Particle Size Distribution of crystalline BisEDT samples

| Sample | D(10) μm | D(50) μm | D(90) μm | D[4,3] μm | Conditions |
|---|---|---|---|---|---|
| 1 | 0.80 | 2.4 | 5.9 | 2.9 | Dalton Synthesis |
| | | | | | Conditions |
| 2 | 0.58 | 1.7 | 3.9 | 2.0 | Dalton Synthesis |
| | | | | | Conditions |
| 3 | 0.59 | 1.9 | 4.5 | 2.3 | 30° C., 5 h addition of 1,2- |
| | | | | | ethane dithiol via addition |
| | | | | | funnel |
| 4 | 0.44 | 1.48 | 3.1 | 1.7 | 30° C., 4 hour addition of |
| | | | | | 1,2-ethane dithiol via |
| | | | | | syringe pump |
| 5 | 0.33 | 0.72 | 1.6 | 0.86 | 20° C., 1 h addition of 1,2- |
| | | | | | ethane dithiol via addition |
| | | | | | funnel |
| 6 | 0.34 | 0.87 | 1.8 | 0.98 | 20° C., 4 h addition of 1,2- |
| | | | | | ethane dithiol via addition |
| | | | | | funnel |
| 7 | 0.39 | 1.3 | 1.6 | 1.4 | 20° C., 1 hour addition of |
| | | | | | 1,2-ethane dithiol via |
| | | | | | syringe pump. Sample |
| | | | | | slurried in EtOH. Cloth |
| | | | | | filtration |
| 8 | 0.36 | 1.0 | 1.8 | 1.0 | 20° C. 1 hour addition of |
| | | | | | 1,2-ethane dithiol via |
| | | | | | syringe pump. Sample |
| | | | | | slurried in MeOH. Cloth |
| | | | | | filtration |
| 9 | 0.67 | 1.0 | 1.9 | 1.1 | 20° C., 1 hour addition of |
| | | | | | 1,2-ethane dithiol via |
| | | | | | syringe pump. Sample |
| | | | | | slurried in Abs. MeOH. |
| | | | | | Cloth filtration |
| 10 | 0.36 | 0.88 | 1.7 | 0.97 | 20° C., 1 hour addition of |
| | | | | | 1,2-ethane dithiol via |
| | | | | | syringe pump. Sample |
| | | | | | slurried in IPA. Cloth |
| | | | | | filtration |
| 11 | 0.38 | 1.2 | 2.4 | 1.4 | 15° C. 1.5 hour addition of |
| | | | | | 1,2-ethane dithiol via |
| | | | | | syringe pump. Cloth |
| | | | | | filtration |
| 12 | 0.37 | 1.2 | 2.4 | 1.3 | 20° C., 1.25 hour addition |
| | | | | | of 1,2-ethane via syringe |
| | | | | | pump |
| 13 | 0.36 | 0.98 | 2.1 | 1,2 | 10° C., 1 h addition of 1,2- |
| | | | | | ethane dithiol via syringe |
| | | | | | pump |
| 14 | 0.36 | 1.0 | 2.1 | 1.2 | 10° C. 1 hour addition of |
| | | | | | 1,2-ethane dithiol via |
| | | | | | syringe pump. Cloth |
| | | | | | filtration |
| 15 | 0.32 | 0.72 | 1.6 | 0.86 | 10° C., 4 hours addition of |
| | | | | | 1,2-ethane dithiol via |
| | | | | | syringe pump. Cloth |
| | | | | | filtration. |

### Example 5. A Phase 1b/2a Randomized, Double-Blind, Placebo-Controlled, Multi-Center Study to Assess the Efficacy of Topical BisEDT in Patients with Moderate to Severe Diabetic Foot Infection

Adjunctive local administration of BisEDT was tested for its efficacy in resolving both infection and critical colonization, thereby removing these barriers to wound repair and closure. This represents a fundamental, clinically meaningful benefit.

Topical BisEDT suspension and placebo were used in combination with SOC. systemic antibiotics (such as levofloxacin) for a duration of 2-3 weeks. This resulted in a similar proportion of patients in the BisEDT treated groups as compared to placebo who were clinically cured at the test of cure visit, which occurred two weeks following the end of treatment. The 3-D photographic data of wound size suggested that BisEDT may be beneficial in facilitating wound repair and closure, as compared to placebo. A larger proportion of BisEDT treated subjects had a >50% reduction in wound surface area from baseline at the end of treatment (Week 2-3), test of cure (Week 4-5), and at the end of study (Week 8-9) (Table 2). BisEDT, when used in conjunction with systemic antibiotics, for a duration of four weeks (3× per week) may be beneficial in resolving both infection and critical colonization, therefore enabling wound repair and closure, a fundamental, clinically meaningful benefit.

TABLE 5
Proportion of patients with a >50% reduction in wound surface
area in Study topical BisEDT suspension, using 3D Digital Photographs

| | Treatment Group | | | | |
|---|---|---|---|---|---|
| | BisEDT | BisEDT | BisEDT | BisEDT | |
| | 3 μg/cm² | 7.5 μg/cm² | 15 μg/cm² | Pooled | Placebo |
| Time Point | (N = 12) | (N = 12) | (N = 15) | (N = 39) | (N = 13) |
| End of | 42% | 55% | 33% | 42% | 23% |
| Treatment | | | | | |
| (Week 2-3) | | | | | |
| Test of Cure | 55% | 67% | 57% | 59% | 39% |
| (Week 4-5) | | | | | |
| End of Study | 55% | 63% | 64% | 61% | 42% |
| (Week 8-9) | | | | | |

### Example 6: Sputum Studies

Bacterial killing curves with BisEDT and BisBDT were performed in the presence of three cystic fibrosis patient sputum in order to determine whether the test compounds are potentially inactivated by sputum. The assay is described in King P, Lomovskaya O, Griffith D C, Burns J L, Dudley M N. In vitro pharmacodynamics of levofloxacin and other aerosolized antibiotics under multiple conditions relevant to chronic pulmonary infection in cystic fibrosis. Antimicrob Agents Chemother, 54:143-8, 2010.

Sputum was collected from cystic fibrosis (CF) patient volunteers without recent antibiotic exposure after appropriate IRB approval. Sputum was sterilized by UV irradiation and sterilization was confirmed by culture. An overnight culture of Pseudomonas aeruginosa strain PA01 was used to inoculate fresh cultures in cation-adjusted Mueller-Hinton broth or cation-adjusted Mueller-Hinton broth plus 10% CF patient sputum. Drugs were added to individual culture tubes with and without sputum at the following final concentrations:
BisEDT: 0.1 μg/mL, 2 μg/mL, and 20 μg/mL
BisBDT: 0.1 μg/mL. 4 μg/mL, and 20 μg/mL

Tobramycin at 1 μg/mL was used as a comparator drug control known to be partially inactivated by patient sputum. Cultures were incubated with shaking at 37° C. and aliquots were removed every hour for quantitation of colony forming units per mL (CFU/mL) by serial dilution and plating on tryptic soy agar for a total of 6 hours. CFU were counted after incubation of the plates overnight at 37° C.

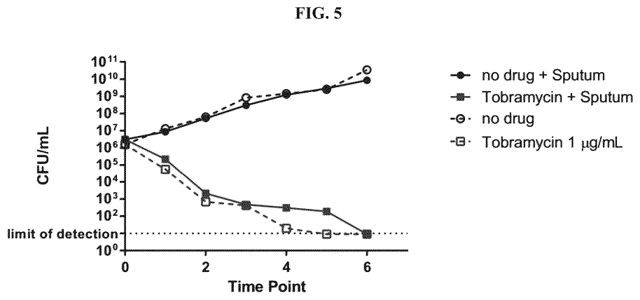

The controls for this assay performed as expected. The growth of PA01 was not obviously inhibited or enhanced by the addition of sterile patient sputum in the absence of additional drug to bacterial cultures (FIG. 5, closed and open circles). As shown, sputum partially inhibits the bacterial killing activity of tobramycin, with approximately 0.5-1 log CFU/mL higher at most time points in cultures with sputum compared to cultures without sputum.

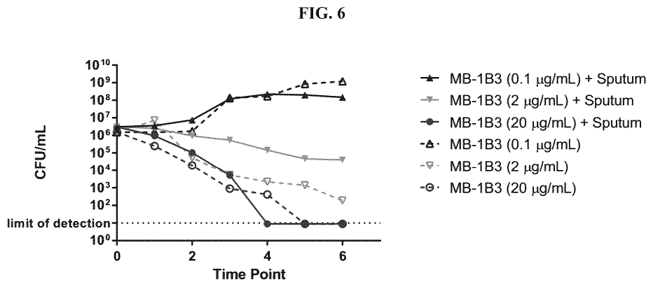

The bactericidal activity of BisEDT appears to be partially inhibited by CF patient sputum based on this assay (FIG. 6). BisEDT is not bactericidal against PA01 at 0.1 μg/mL. With BisEDT at 2 μg/mL, the addition of sputum reduces killing by approximately 1-2 log CFU/mL at 3 to 6 hours. When the concentration of BisEDT is further increased to 20 μg/mL the inhibition of bacterial killing by sputum is less pronounced, with killing in the presence of sputum lagging by only 0.5-1 log CFU/mL behind cultures without sputum at early time points (1-3 hours). Eventually, with this highest dose tested, bacteria in the culture with sputum is reduced below the limit of detection 1 hour earlier than the sample without sputum, suggesting that at higher drug concentrations inhibition of BisEDT by sputum is largely overcome.

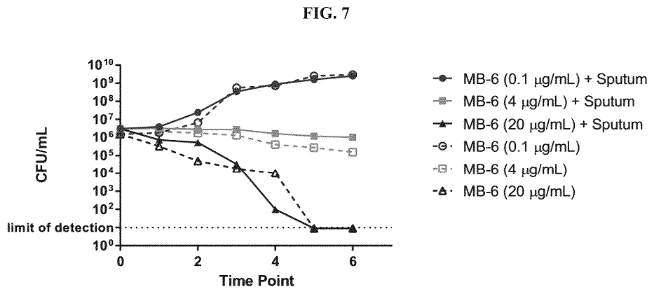

Similarly, the bactericidal activity of BisBDT appears to be partially inhibited by CF patient sputum (FIG. 7). BisBDT at 0.1 μg/mL is not bactericidal against PA01. In the absence of sputum, 4 μg/mL BisBDT demonstrated very slow bactericidal activity against PA01, with killing of only about 1 log over the 6 hour assay; cultures with sputum demonstrated approximately 0.5-0.8 log CFU/mL more surviving at 3-6 hours. At the highest concentration of BisBDT tested, 20 μg/mL, there is an initial lag in killing in PA01 at 1-2 hours with the addition of sputum, but both samples with and without sputum are sterilized below the limit of detection by 5 hours.

Both compounds BisEDT and BisBDT are bactericidal against Pseudomonas aeruginosa strain PA01 in this assay, and this bactericidal activity is partially inhibited in the presence of CF patient sputum. This partial inhibition of bactericidal activity can be overcome by increased concentration of the test compounds. Thus, a higher concentration of bismuth-thiol compound may be needed in areas where sputum is present compared to bodily compartments without sputum.

### Example 8: In Vivo Studies

Objectives: The primary objective of this study is to assess the tolerance of BisEDT (Dalton Pharma Services; Lot #ED268-1-11-01 stored at room temperature) following nose only inhalation exposure in F344 rats. Animals will be exposed to differing concentrations (low, middle, high) for up to 180 minutes. Blood will be collected at predetermined timepoints to analyze for systemic presence of BisEDT. At the conclusion of the study (24 hr post exposure) animals will be euthanized and undergo necropsy where lungs will be lavaged and collected for potential, future analysis. Additional respiratory tract related tissues such as the nasal cavity, larynx, pharynx, nasopharynx, trachea, bronchus, and carina will also be collected. Lavage fluid will be analyzed for clinical chemistry and hematology parameters including cell counts and differentials. Liver, kidney, esophagus, stomach, small intestine, and large intestine will also be collected for potential, future analysis.

Animals: Male and female F344 rats provided by Charles River Labs, Wilmington, MA were used in this study. The rats were approximately 7-9 weeks old at the time of arrival and 8-10 weeks at study initiation. Body weights of individual animals were ±20% of the group mean. Animals will be uniquely identified by numeric tail markings (made with indelible ink such as a Sharpie®) for body weights, randomization, and treatment administration. Color coded cage cards will also be placed on the cages. A total of forty (40) male and female F344 rats (20 M/20 F) (including spares) were ordered for the study. Thirty six (36) animals were randomized into 3 study groups each consisting of 12 animals per group (6 M/6 F). The remaining 4 animals (2 M 12 F) were spares. Upon removal from quarantine, thirty six (36) rats will be randomized into 3 study groups each consisting of 12 animals (6 M/6 F) per group by body weight stratification. Unused spares will be either euthanized or conveyed to another approved study protocol. Prior to the start of exposures, animals will be conditioned to nose-only exposure tubes.

Animal Husbandry: Animals will be housed for a minimum of 7 days, up to 2 per cage in polycarbonate shoebox cages with Alpha Dri or hardwood chip bedding. Caging and bedding were autoclaved. Prior to injection, animals were introduced at least once to restraint tubes that will be used for tail vein dosing. Animal feed was 2016C Harlan Global Certified Rodent Chow, (Harlan Tekland, Madison, WI), unlimited access except during study procedures. Each batch of feed wass analyzed for contaminants by the manufacturer and will be used within the manufacturer's designated shelf-life. Animals were provided municipal water (filtered at 5, 1, and 0.2 p.m), unlimited access except during study procedures. Only healthy animals were used in this study. A laboratory animal veterinarian or designee visually examined the animals before release from quarantine.

Environmental Conditions: The targeted conditions for animal room environment and photoperiod will be as follows: Temperature: 18-26° C.; Humidity: 30-70%; Light Cycle: 12-h. Light, humidity, and temperature excursions are defined as a sustained reading that falls out of the specified range for more than 3 hours.

Experimental Design: The experimental design for this study was: Group 1: Low dose; inhalation; 6 males and 6 females; blood collection at 0.5, 2 hr and 8 hr post exposure and 24 hr (terminal); and necropsy at 24 hours-post inhalation. Group 2: Mid dose; inhalation; 6 males and 6 females; blood collection at 0.5, 2 hr and 8 hr post exposure and 24 hr (terminal); and necropsy at 24 hours-post inhalation. Group 3: high dose; inhalation; 6 males and 6 females; blood collection at 0.5, 2 hr and 8 hr post exposure and 24 hr (terminal); and necropsy at 24 hours-post inhalation. Animals were weighed and randomized into study groups following the quarantine period. Animals were broken into 3 groups each consisting of 6 M and 6 F per group. Groups were exposed to three different dose levels of BisEDT. The initial exposure was to a formulation concentration of 100 mg/mL for an exposure time of 60 minutes; based on method development this is expected to result in a dose of 3 mg/kg. The maximum duration for exposures was 180 minutes and the maximum formulation concentration was 100 mg/mL.

Following exposure, non-terminal blood collections were performed on 2 animals per sex at 0.5, 2, and 8 hours post exposure. At the conclusion of the study (24 hr post exposure) animals were euthanized and underwent necropsy. The right lung was lavaged, flash frozen, and collected for potential, future analysis. The left lung was flash frozen for potential, future analysis. Additional respiratory tract related tissues such as the nasal cavity, larynx, pharynx, nasopharynx, trachea, bronchus, and carina were collected. Lavage fluid was analyzed for clinical chemistry and hematology parameters including cell counts and differentials. Liver, kidney, esophagus, stomach, small intestine, and large intestine were collected for potential, future analysis. These organ tissue samples were flash frozen and stored pending study completion. The lavage fluid were analyzed for clinical pathology and hematology (including cell counts and differentials). A maximum whole blood collection was also collected at necropsy. Blood samples were analyzed for clinical chemistry and hematology as well as an aliquot snap frozen for potential analysis for BisEDT.

Inhalation Exposure: The initial exposure of BisEDT (Bismuth-1,2-ethanedithiol) was formulated as a solution at 100 mg/mL in suspension in 0.5% Tween 80, 10 mM sodium phosphate, pH 7.4, in NaCl (adjusted to approximately 300 mOsm). Aerosols were generated with a commercial compressed air jet nebulizer, Pad LC Plus, operated with an inlet pressure of 20 psi. A schematic is shown in FIG. 1. The aerosols were transitioned into a rodent nose-only inhalation exposure system. The exposure system was operated with an inlet air flow of −5.2/min and an exhaust air flow of −5 L/min. This resulted in 0.31 L/min to each port which is slightly greater than 1.5× the respiratory minute volume of a rat.

Exposure Concentration and Particle Size Monitoring: Aerosol concentration was monitored at the breathing zone by collection onto a GF/a filter. The filters were analyzed via differential mass and other methods known in the art. Aerosol particle size was measured using a TSI Aerodynamic Particle Sizer (Model 3321, TSI, Inc., Shoreview, MN) or an In-Tox Mercer 2.0 L/min cascade impactor.

Observations and measurements: Observations were documented in Provantis database or the Animal Management System (AMS, LRRI, Albuquerque).

Clinical Observations and Mortality/Morbidity: Detailed clinical observations were recorded starting on dose day with observations recorded prior to exposure, during exposure, after exposure as the animals are returned to their home cages, and in the afternoon post-exposure. Observations will be recorded according to a standard lexicon (SOP TXP-1532-Pharmacologic and Toxicologic Observations of Experimental Animals). General observations include but are not limited to apnea, labored breathing, malaise, marked nasal discharge, etc. Special attention was paid to clinical signs associated with the respiratory tract. Animals showing severe signs of distress were euthanized immediately at the discretion of the Study Director in consultation with veterinary staff. Examinations were also oriented toward (1) identifying dead, weak, or moribund animals, and (2) documenting the onset and progression of any abnormal clinical signs. Moribund or dead animals were necropsied as soon as possible after being found but in no case later than 16 hours after being found.

Body Weights: All animals were weighed after release from quarantine and that weight will be the pre-study body weight used to randomize animals into dose groups. Body weights were collected in the morning prior to exposure and again at necropsy.

Blood Collection for Clinical Chemistry and Hematology: Blood samples for hematology and clinical chemistry were collected during necropsy. For Complete Blood Count (CBC) with absolute differentials, whole blood (target 0.5 mL) was collected and placed into tubes containing tripotassium ethylenediaminetetraacetate (K₃EDTA) as an anticoagulant. Hematology samples was analyzed by automated (ADVIA™ 120 Hematology System, Siemens Medical Solutions Diagnostics, Tarrytown, NY) analyses. The parameters for hematology are: Red Blood Cell Count (RBC) 10⁶/μL; Hemoglobin (HGB) g/dL; Hematocrit (HCT) %; Mean Corpuscular Volume (MCV) fL; Concentration (MCHC) g/dL; Mean Corpuscular Hemoglobin (MCH) pg; Platelet Count (PLT) (10³/μL); Percent Reticulocytes (RETIC) % RBC; White Blood Cell Count (WBC) 10³/μL; Neutrophils (PMN) 10³/μL; Lymphocytes (LYM) 10³/μL; Monocytes (MONO) 10³/μL; Eosinophils (EOS) 10³/μL; Basophils (BASO) 10³/μL; Large Unstained Cells (LUC) 10³/μL.

For clinical chemistry analyses, whole blood (≥0.5 mL) was placed into serum separator or clot tube for centrifugation and separation into cellular and serum fractions. Serum samples were analyzed on a Hitachi Modular Analytics Clinical Chemistry System (Roche Diagnostics, Indianapolis, IN). The clinical chemistry parameters measured or calculated are: Alanine Aminotransferase (Alanine Transaminase)-Serum (ALT) IU/L; Albumin (ALB) g/dL; Aspartate Aminotransferase (Aspartate Transaminase)-Serum (AST) IU/L; Bilirubin (Total) (BILI-T) mg/dL; Blood Urea Nitrogen (BUN) mg/dL; Calcium (CA) mg/dL; Chloride (Serum) (CL-S) mmol/L; Cholesterol (Total) (CHOL) mg/dL; Creatinine (Serum)(CRE-S) mg/dL; Glucose (GLU) mg/dL; Gamma Glutamyltransferase (GGT) IU/L; Alkaline Phosphatase (ALP) IU/L; Phosphate (PHOS) mg/dL; Potassium (Serum) (K-S) mmol/L; Protein (Total) (TP) g/dL; Sodium (Serum) (NA-S) mmol/L; Triglycerides (TRIG) mg/dL; Albumin/Globulin (A/G) no units; Blood Urea Nitrogen/Creatinine (BUN/CRE) no units; Globulin (GLOBN) g/dL.

Hematology and clinical chemistry evaluations were performed on all study animals for which adequate sample volumes are obtained and for which no analytical problems are encountered. If target collection volumes are not obtained or if evaluations are not performed, a reason and notation will be included in the raw data. The remaining blood samples or serum will be discarded after the analyses.

Blood Collection for Bioanalytical Analysis: Following exposure, non-terminal blood collections were performed on 2 animals per sex at 0.5, 2, and 8 hours post exposure. Approximately 1 mL of systemic whole blood was collected by jugular vein into tubes containing K₃EDTA as an anticoagulant. The tubes were flash frozen with liquid nitrogen stored without processing at −70 to −90° C. until shipping for analysis using ICP-MS assay for quantitation of bismuth as a surrogate for BisEDT.

Euthanasia and necropsy: Animals were fasted overnight prior to scheduled necropsy (24 hr post exposure). At scheduled necropsy or in cases of morbidity, animals were euthanized by intraperitoneal injection of an overdose of a barbiturate-based sedative. Detailed gross necropsies were performed on all animals (found dead, moribund, or scheduled necropsy) and consisted of a complete external and internal examination including body orifices and cranial, thoracic, and abdominal organs and tissues. All gross findings will be recorded in descriptive terms. Whole blood was collected for bioanalytical analysis, hematology, and clinical chemistry at necropsy. Lungs collected and weighed. The left lobe were tied off and flash frozen for potential, future analysis. The right lobes were lavaged 3× using 4 mL/lavage of phosphate buffered saline (PBS). After lavage is complete the right lobes were flash frozen in liquid nitrogen for potential, future analysis.

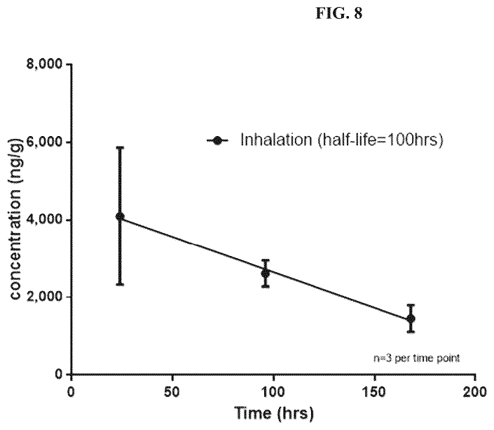

This Example describes the results of BisEDT single-dose rat PK studies comparing inhalation, IV, and oral dosing. The primary takeaways are that (1) BisEDT remains in lung tissue after inhalation dosing with a half-life of about 4 days (FIG. 8). (2) there are very low, but sustained and measurable blood concentrations after oral dosing; (3) IV dosing appears to follow a biphasic pattern with initial distribution into tissues for 18 hours, followed by a slow systemic elimination phase; (4) BisEDT does not appreciably partition into lung tissue after IV or oral dosing—no lung levels after oral and low lung levels detected after IV dosing (<5% vs inhalation group) and levels dropped rapidly with about 24 hour half-life. This indicates that systemic BisEDT does not partition into lung tissue and that lung levels measured after inhalation dosing are due to deposited drug particles on the apical surface; (5) after inhalation dosing, there is sustained, moderate, blood exposure with relatively stable concentrations across time, indicating the drug in the lungs is acting as a depot; (6) BisEDT was tolerated at 100 μg/kg inhalation and IV and 250 μg/kg oral. Based on these data, low doses given daily or every other day are likely to provide very stable drug levels in tissue and blood (small differences between min and max). If the safety margin is demonstrated to be large enough during GLP toxicology and/or clinical studies, it is possible to lengthen the dosing interval and the increase the dose accordingly. Increasing the dose and interval leads to larger fluctuations in tissue and blood levels between peak (after dosing) and trough levels (prior to dosing).

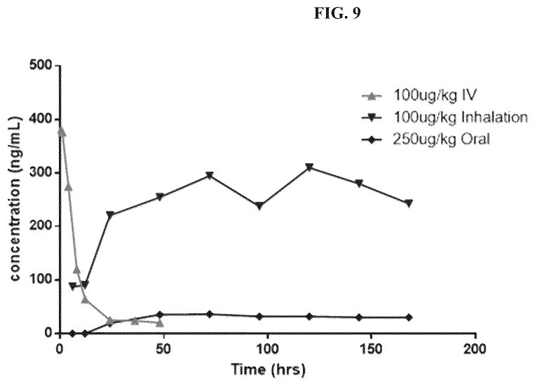

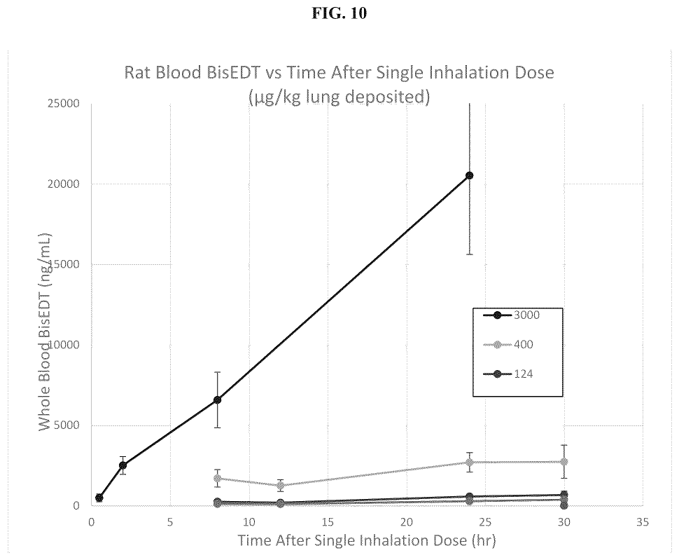

Based on existing in vivo and in vitro toxicology data as well as rat PK and in vitro MIC data, BisEDT suspension for inhalation can be reliably administered at doses providing efficacious lung levels that are tolerated, and it is therefore a viable clinical development candidate. As noted above, BisEDT remains in lung tissue long after inhalation dosing with a half-life of about 4 days (FIG. 9). 24 hours after single inhalation dose 4,093 ng/g was measured in the lung tissue (which equates to 123 μg/mL in lung fluid) and 4 days (96 hrs) after single dose 2,600 ng/g in lung tissue (which equates to 78 μg/mL in lung fluid). Without being bound by any particular theory, it is believed BisEDT's low solubility provides slow dissolution and long exposure on lung surface fluids with limited systemic exposure via diffusion through lung epithelium. The lung appears to act as a depot for slow systemic exposure with about 10× lower systemic levels than seen in the lung. The terminal phase of the IV data appears to show slow elimination after initial tissue distribution (FIG. 10).

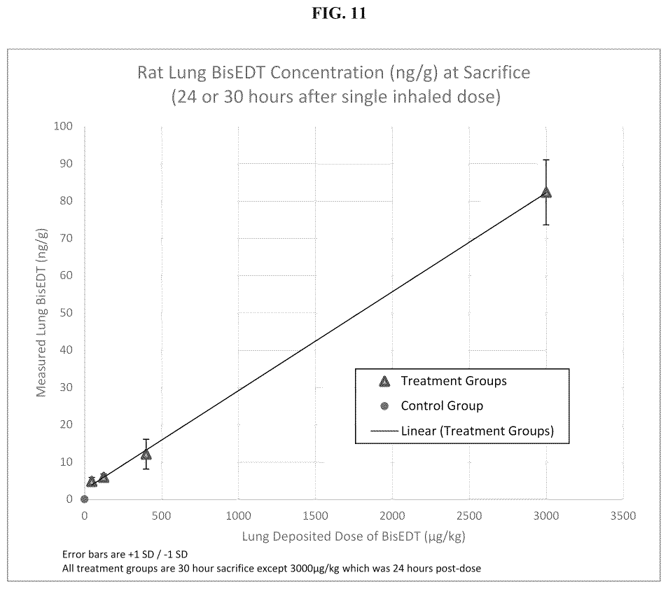

Tables 6 and 7 below show whole blood BisEDT versus time data and lung BisEDT concentration at sacrifice time data respectively. These results are also shown graphically in FIG. 11.

TABLE 6
Whole Blood BisEDT versus Time Data

| | | Mean | |
|---|---|---|---|
| | | BisEDT | |
| Dose | Nominal | Conc | |
| (μg/kg) | Time (hr) | (ng/mL) | STDEV |
| 3000 | 1 | 485 | 244 |
| 3000 | 2 | 2504 | 547 |
| 3000 | 8 | 6570 | 1729 |
| 3000 | 24 | 20525 | 4907 |
| 47 | 8 | 114 | 64 |
| 47 | 12 | 98 | 35 |
| 47 | 24 | 282 | 84 |
| 47 | 30 | 373 | 201 |
| 400 | 8 | 1701 | 542 |
| 400 | 12 | 1252 | 368 |
| 400 | 24 | 2700 | 607 |
| 400 | 30 | 2738 | 1028 |
| 124 | 8 | 247 | 3 |
| 124 | 12 | 187 | 78 |
| 124 | 24 | 571 | 93 |
| 124 | 30 | 668 | 239 |
| 0 | 30 | 0 | N/A |
| 0 | 30 | 0 | N/A |
| 0 | 30 | 0 | N/A |

TABLE 7
Lung BisEDT Conc. at Sacrifice

| | | Mean | |
|---|---|---|---|
| | | BisEDT | |
| Dose | Sac time | Conc | |
| (μg/kg) | (hr) | (ng/g) | STDEV |
| 3000 | 24 | 82.3 | 8.7 |
| 400 | 30 | 12.1 | 4 |
| 124 | 30 | 5.9 | 0.9 |
| 47 | 30 | 4.8 | 1.1 |
| 0 | 30 | 0 | N/A |

The efficacy of BisEDT in reducing pulmonary bacterial burden associated with pulmonary infection in a rat model of pulmonary Pseudomonas aeruginosa infection was evaluated. The results follow.

Aerosol concentration: The results of aerosol concentration (gravimetric and chemistry) are presented in Table 8. No BisEDT was detected by chemical analysis of the vehicle, as expected. Gravimetric analysis is listed. The positive control (Tobramycin) concentration was determined gravimetrically and all exposure atmospheres varied by less than 10% through both cohorts and all exposure days. The test article concentration, determined by chemical analysis, varied by less than 5.1% for all exposure cohorts and days and was 11.5 μg/L.

TABLE 8
Average concentration of exposure atmospheres

| | Analysis | Concentration | | |
|---|---|---|---|---|
| Group | Method | Average | | RSD |
| Vehicle | Gravimetric | 0.11 ± 0.02 | mg/L | 14.8% |
| Tobramycin | Gravimetric | 1.26 ± 0.10 | mg/L | 8.3% |
| BisEDT | Chemical | 11.5 ± 0.6 | μg/L | 5.1% |

Dose Delivered: Tables 9 and 10 describe the calculated presented and theoretical deposited doses delivered to the animals in this study. The presented dose is defined as the total inhaled amount of material, comprising material deposited in the sinuses, throat, oropharyngeal region, lung, as well as exhaled material. The theoretical deposited dose, or amount of material that is actually deposited on the surface of the lung, is considered to be 10% of the presented dose in rats (Inhal Toxicol. 2008 October; 20(13):1179-89. doi: 10.1080/08958370802207318, which is hereby incorporated by reference in its entirety). Each animal's current body weight and exposure condition were factored into the equations above for dose determination. Group 3 and Group 4 exposures were 13 minutes and 30 minutes in duration, respectively. The vehicle filters were analyzed chemically for BisEDT and all filters were below detection limits. The positive control (tobramycin) filters were analyzed gravimetrically and the average presented dose for all cohorts and days was 29.0 mg/kg, higher than the 20 mg/kg value specified in the study protocol. The average presented doses for the low and high concentration groups of BisEDT were 0.114 mg/kg and 0.264 mg/kg, respectively. The study protocol called for doses of 0.1 mg/kg and 0.25 mg/kg for those groups. Details of doses received by exposure groups by day and cohort are listed in Tables 9 and 10 below.

TABLE 9
Presented Dose Summary

| | | Cohort 1 Dose | Cohort 2 Dose |
|---|---|---|---|
| Group | Day | (mg/kg) | (mg/kg) |
| Vehicle | 0 | BDL | BDL |
| Vehicle | 2 | BDL | BDL |
| Vehicle | 4 | BDL | BDL |
| Tobramycin | 1 | 31.0 ± 3.4 | 28.9 ± 1.8 |
| Tobramycin | 2 | 29.0 ± 1.9 | 27.9 ± 0.8 |
| Tobramycin | 3 | 27.9 ± 0.7 | 29.3 ± 1.8 |
| Tobramycin | 4 | 29.3 ± 1.7 | 28.5 ± 2.3 |
| BisEDT | 0 | 0.121 ± 0.002 | 0.110 ± 0.002 |
| (0.1 mg/kg) | | | |
| BisEDT | 2 | 0.120 ± 0.002 | 0.120 ± 0.002 |
| (0.1 mg/kg) | | | |
| BisEDT | 4 | 0.101 ± 0.002 | 0.114 ± 0.002 |
| (0.1 mg/kg) | | | |
| BisEDT | −1 | 0.263 ± 0.005 | 0.264 ± 0.003 |
| 0.25 mg/kg | | | |

TABLE 10
Theoretical Deposited Dose Summary

| Group | Day | Cohort 1 Dose | | Cohort 2 Dose | |
|---|---|---|---|---|---|
| Vehicle | 0 | BDL | | BDL | |
| Vehicle | 2 | BDL | | BDL | |
| Vehicle | 4 | BDL | | BDL | |
| Tobramycin | 1 | 3.1 | mg/kg | 2.9 | mg/kg |
| Tobramycin | 2 | 2.9 | mg/kg | 2.8 | mg/kg |
| Tobramycin | 3 | 2.8 | mg/kg | 2.9 | mg/kg |
| Tobramycin | 4 | 2.9 | mg/kg | 2.9 | mg/kg |
| BisEDT | 0 | 12.1 | μg/kg | 11.0 | μg/kg |
| (0.1 mg/kg) | | | | | |
| BisEDT | 2 | 12.0 | μg/kg | 12.0 | μg/kg |
| (0.1 mg/kg) | | | | | |
| BisEDT | 4 | 10.1 | μg/kg | 11.4 | μg/kg |
| (0.1 mg/kg) | | | | | |
| BisEDT | −1 | 26.5 | μg/kg | 26.4 | μg/kg |

0.25 mg/kg

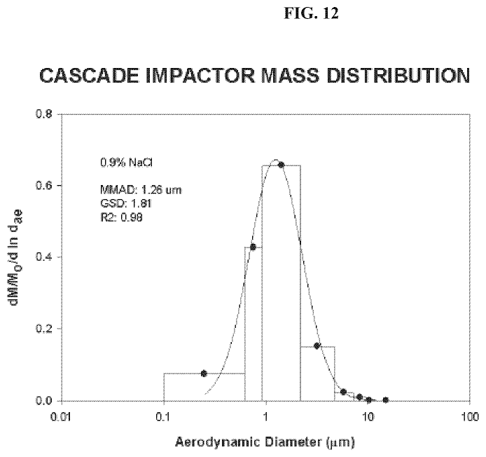

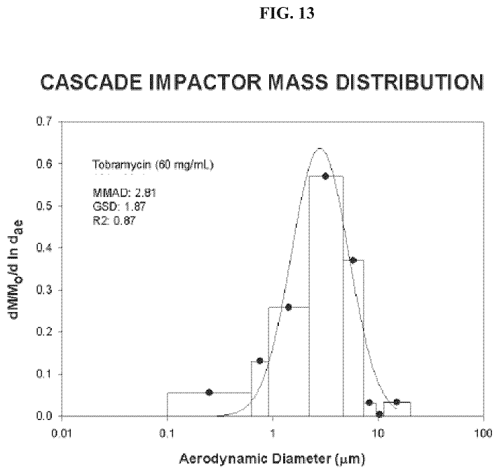

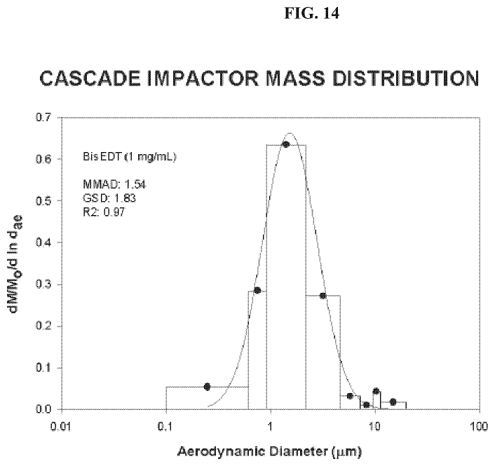

Particle Size Distribution: Particle size distributions (FIGS. 12-14) for each exposure condition was determined using an In-Tox Mercer Impactor. FIG. 13 shows the aerosol size distribution of BisEDT measured during the study. Table 11 provides summaries of particle size distributions (PSD) and geometric standard deviations (GSD) for each exposure type.

TABLE 11
Summary of size distributions for different exposure conditions

| Exposure Conditions | MMAD (μm) | GSD (μm) |
|---|---|---|
| Vehicle | 1.26 | 1.81 |
| Tobramycin | 2.81 | 1.87 |
| BisEDT | 1.54 | 1.83 |

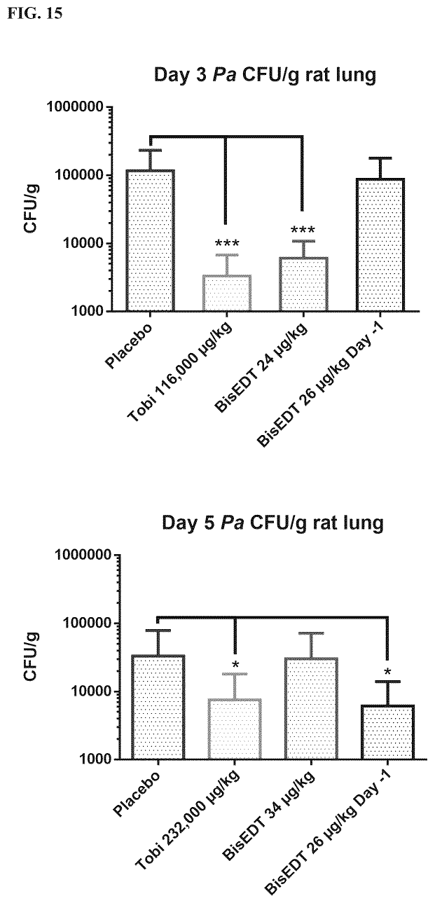

FIG. 15 shows rat efficacy figures showing cumulative (total) administered dose (lung deposited) at days 3 and 5. As can be seen from this figure, the mass ratio of delivered drug is staggering compared to Tobi.

Conclusion: Animals were divided into four experiment inhalational treatment groups: 1) Group 1, a negative control group treated with saline on Days 0, 2, and 4; 2) Group 2, a positive control group treated with tobramycin (target of 20 mg/kg, BID, Days 1-4); 3) Group 3, BisEDT (target of 0.1 mg/kg, QD, Days 0, 2, 4); and Group 4, BisEDT (target of 0.25 mg/kg, once, Day −1). Animals were further divided into two cohorts, separated by one day, to accommodate the challenge and treatment of the number of animals. All animals were exposed per protocol except Group 1 and 3, cohort 2. These animals were exposed on Days 0, 1, and 4 instead of on Days 0, 2, 4.

BisEDT was not detected in animals receiving saline (negative control). Animals treated twice daily (8 doses) with tobramycin received an average dose of 29.0 mg/kg. This dose represents 145% of the targeted dose. As tobramycin is used as a positive control to ensure the model function rather than as a direct comparison, or as a competitor, for the activity of the test article, the increased dose does not impact the study. Animals treated with three targeted doses of 0.1 mg/kg BisEDT received an average of 0.114 mg/kg which represents 114% of the targeted dose. For Group 3 (targeted dose of 0.1 mg/kg), cohort 1 and 2 average doses for each day were, 0.114 and 0.115, respectively. An unpaired t-test (GraphPad Prism 5.0) comparing average dose over the three treatment days demonstrated the differences between cohorts to not be significant (p-value=0.93). Animals which received the single prophylactic targeted dose of 0.25 mg/kg received nearly identical doses of 0.263 mg/kg (cohort 1) and 0.264 mg/kg (cohort 2) of BisEDT, or 105% and 106%, respectively of the targeted dose.

The particles generated for exposure were considered respirable. The positive control (tobramycin) average MMAD of 2.81 μm with a GSD 1.87 μm. Aerosol particle MMAD of negative control (saline) treatment and test article (BisEDT) treatment were 1.26 μm and 1.54 μm with a GSD of 1.81 and 1.83, respectively.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. An amorphous form of bismuth-1,2-ethanedithiol (BisEDT).

2. The amorphous form of BisEDT according to claim 1, wherein its X-ray powder diffraction pattern does not contain any distinct peaks.

3. The amorphous form of BisEDT according to claim 1 or claim 2, wherein its X-ray powder diffraction pattern is substantially similar to FIG. 1.

4. The amorphous form of BisEDT according to any one of claims 1-3, wherein its differential scanning calorimetry thermogram comprises an exothermic peak at about 168° C.

5. The amorphous form of BisEDT according to claim 4, wherein its differential scanning calorimetry thermogram further comprises an endotherm at about 64° C. and/or an endotherm peak at about 112° C. and/or an exotherm peak at about 145° C.

6. The amorphous form of BisEDT according to any of the preceding claims, wherein its differential scanning calorimetry thermogram is substantially similar to FIG. 2.

7. The amorphous form of BisEDT according to any of the preceding claims having a glass transition at about 101° C.

8. The amorphous form of any one of claims 1-7, wherein the amorphous form is at least 90% pure.

9. The amorphous form of claim 8, wherein the amorphous form is at least 95% pure.

10. The amorphous form of claim 9, wherein the amorphous form is at least 98% pure.

11. A composition comprising an amorphous from of bismuth-1,2-ethanedithiol (BisEDT).

12. The composition of claim 11, wherein the composition comprises at least one pharmaceutically acceptable carrier.

13. The composition of claim 11, wherein the composition comprises BisEDT in a suspension.

14. A method of treating, managing, preventing, or lessening the severity of symptoms and infections associated with one or more pulmonary diseases or infections in a subject, the method comprising administering to the subject a bismuth-thiol (BT) composition that comprises amorphous BisEDT according to any one of claims 1-10 suspended therein, wherein administering the BT composition is via inhalation, orally or nasally, using an aerosol device.

15. The method of claim 14, wherein the method is treating, managing or lessening the severity of cystic fibrosis (CF) symptoms and infections in a subject.

16. A method for healing a wound in a subject having a diabetic foot infection or other chronic wound infection, comprising administering the subject a therapeutically effective amount of a composition comprising amorphous BisEDT according to any one of claims 1-10.

17. The method of claim 16, wherein the wound is a diabetic foot ulcer.

18. A method of making an amorphous form of BisEDT according to any one of claims 1-10, comprising
(a) mixing an acidic aqueous solution that comprises a bismuth salt, with a solvent selected from the group consisting of acetone, acetonitrile, 1,2-dichloroethane, dimethyl sulfoxide, ethyl acetate, isopropanol, methyl tert-butyl ether, and mixtures thereof;
(b) combing the product of (a) with a solution of 1,2-ethanedithiol in a solvent selected from the group consisting of acetone, acetonitrile, 1,2-dichloroethane, dimethyl sulfoxide, ethyl acetate, isopropanol, methyl tert-butyl ether, and mixtures thereof, under conditions and for a time sufficient for formation of a precipitate which comprises the amorphous form of BisEDT.

19. The method of claim 18, further comprising recovering the precipitate to remove impurities.

20. The method of claim 18 or 19, wherein the bismuth salt is Bi(NO₃)₃.

21. The method of any one of claims 18-20, wherein 1,2-ethanedithiol is at a concentration of from about 1% wt/vol to about 20% wt/vol prior to step (b).

22. The method of any one of claims 18-21, wherein the acidic aqueous solution is prepared by mixing an aqueous suspension of either Bi (III) sub-nitrate or Bi (III) nitrate pentahydrate with an acid under conditions and for a time sufficient to form a substantially clear solution.

23. The method of claim 22, wherein the concentration of either Bi (III) sub-nitrate or Bi (III) nitrate pentahydrate in the aqueous solution is from about 100 mg/mL to about 400 mg/mL.

24. The method of claim 22 or claim 23, wherein the acid is 70% HNO₃.

25. The method of any one of claims 22-24, further comprising adding the clear solution to an acidic solution.

26. The method of claim 25, wherein the acidic solution is 5% HNO₃.

27. The method of any one of claims 18-26, wherein step (b) is performed at a temperature ranging from about 20° C. to about 28° C.

28. A method of treating, managing or lessening the severity of cystic fibrosis (CF) symptoms and infections in a subject, the method comprising administering to the subject a bismuth-thiol (BT) composition that comprises amorphous BisEDT according to any one of claims 1-10 suspended therein.

29. The method of claim 14-15 or 28, wherein the BT composition comprises a plurality of microparticles wherein at least 70% of said microparticles having a volumetric mean diameter (VMD) from about 0.01 μm to about 2.5 μm.

30. The method of claim 14-15 or 28 or claim 29, wherein at least 80% of said microparticles having a VMD from about 0.6 μm to about 2.5 μm.

31. The method of any one of claims 14-15 or 28-30, wherein at least 90% of said microparticles having a VMD from about 0.01 μm to about 2.5 μm.

32. The method of any one of claims 14-15 or 28-31, wherein when the BT composition is aerosolized, at least 70% of the aerosolized liquid droplets have a mass median aerodynamic diameter (MMAD) from about 0.03 μm to about 3 μm.

33. The method of any one of claims 14-15 or 28-32, wherein when the BT composition is aerosolized, at least 80% of the aerosolized liquid droplets have a MMAD from about 0.03 μm to about 3 μm.

34. The method of any one of claims 14-15 or 28-33, wherein when the BT composition is aerosolized, at least 90% of the aerosolized liquid droplets have a MMAD from about 0.03 μm to about 3 μm.

35. The method of any one of claims 14-15 or 28-34, wherein the BT composition comprises BisEDT at a concentration greater than about 0.1 mg/mL, about 0.05% to about 1.0% Tween 80®, about 40 mM to 200 mM sodium chloride, and optionally about 2 to 20 mM sodium phosphate at about pH 7.4.

36. The method of claim 35, wherein the range of sodium chloride is about 100 mM to about 200 mM.

37. The method of any one of claims 14-15 or 28-36, wherein the subject has at least one pulmonary infection containing one or more bacterial pathogens and/or fungal pathogens.

38. The method of any one of claims 14-15 or 28-37, wherein the method comprises at least one of: (i) reducing a bacterial biofilm, (ii) impairing growth of a bacterial biofilm, (iii) preventing initial formation of the bacterial biofilm, and/or (iv) preventing reformation of the bacterial biofilm.

39. The method of any one of claims 14-15 or 28-38, wherein the one or more pathogens are selected from Haemophilus influenzae, Pseudomonas aeruginosa, Staphylococcus aureus, Staphylococcus warneri Staphylococcus lugdunensis, Staphylococcus epidermidis, Streptococcus milleri/anginous, Streptococcus pyogenes, non-tuberculosis mycobacteria, Mycobacterium tuberculosis, Burkholderia spp., Achromobacter xylosoxidans, Pandoraea sputorum, Stenotrophomonas maltophilia, Alcaligenes xylosoxidans, Haemophilus pittmaniae, Serratia marcescens, Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, Aspergillus flavus, Morganella morganii, Inquilinus limosus, Ralstonia mannitolilytica, Pandoraea apista, Pandoraea pnomenusa, Pandoraea sputorum, Bdellovibrio bacteriovorus, Bordetella bronchiseptica, Vampirovibrio chlorellavorus, Actinobacter baumanni, Cupriadidus metallidurans, Cupriavidus pauculus, Cupriavidus respiraculi, Delftia acidivordans, Exophilia dermatitidis, Herbaspirillum frisingense, Herbaspirillum seropedicae, Klebsiella pneumoniae, Pandoraea norimbergensis, Pandoraea pulmonicola, Pseudomonas mendocina, Pseudomonas pseudoalcaligenes, Pseudomonas putida, Pseudomonas stutzeri, Ralstonia insidiosa, Ralstonia pickettii, Neisseria gonorrhoeae, NDM-1 positive E. coli, Enterobacter cloaca, Vancomycin-resistant E. faecium, Vancomycin-resistant E. faecalis, E. faecium, E. faecalis, Clindamycin-resistant S. agalactiae, S. agalactiae, Bacteroides fragilis, Clostridium difficile, Streptococcus pneumonia, Moraxella catarrhalis, Haemophilus haemolyticus, Haemophilus parainfluenzae, Chlamydophilia pneumoniae, Mycoplasma pneumoniae, Atopobium, Sphingomonas, Saccharibacteria, Leptotrichia, Capnocytophaga, Oribacterium, Aquabacterium, Lachnoanaerobacudum, Campylobacter, Acinetobacter; Agrobacterium; Bordetella; Brevundimonas; Chryseobacterium; Delftia; Enterobacter; Klebsiella; Pandoraea; Pseudomonas; Ralstonia, and Prevotella.

40. The method of any one of claims 14-15 or 28-29, wherein the one or more pathogens are non-tuberculosis mycobacteria.

41. An aerosol comprising a plurality of dispersed liquid droplets in a gas, said liquid droplets comprising a BT composition comprising amorphous BisEDT according to any one of claims 1-10 suspended therein; and
wherein at least 70% of the liquid droplets have a MMAD from about 0.03 μm to about 3 μm.

42. The aerosol of claim 41, wherein prior to aerosolization, the BT composition comprises a plurality of microparticles wherein at least 70% of said microparticles have a VMD from about 0.01 μm to about 2.5 μm.

43. The aerosol of claim 41 or claim 42, wherein least 80% of the liquid droplets have a MMAD from about 0.03 μm to about 3 μm.

44. The aerosol of any one of claims 41-43, wherein least 90% of the liquid droplets have a MMAD from about 0.03 μm to about 3 μm.

45. The aerosol of any one of claims 41-44, wherein prior to aerosolization, the BT composition comprises a plurality of microparticles wherein at least 80% of said microparticles have a VMD from about 0.01 μm to about 2.5 μm.

46. The aerosol of any one of claims 41-45, wherein prior to aerosolization, the BT composition comprises a plurality of microparticles wherein at least 90% of said microparticles have a VMD from about 0.01 μm to about 2.5 μm.

47. The aerosol of any one of claims 41-46, wherein the droplets further comprise Tween 80 and optionally a buffer at a pH of about 7.4; and/or sodium chloride.

48. The aerosol of claim 47, wherein the range of sodium chloride is about 100 mM to about 200 mM.

49. The aerosol of any one of claims 41-48, wherein a substantial amount of the BisEDT compounds are deposited in the deep lung region.

50. A pharmaceutical composition comprising bismuth-thiol (BT) composition that comprises amorphous BisEDT according to claims 1-10 suspended therein, wherein the BT composition comprises a plurality of microparticles, wherein the D90 of said microparticles is less than or equal to 1.9 μm.

51. The pharmaceutical composition of claim 50, comprising bismuth-thiol (BT) composition comprises BisEDT suspended therein, wherein the BT composition comprises a plurality of microparticles, wherein the D90 of said microparticles is less than or equal to about 1.6 μm.

52. The pharmaceutical composition of claim 50 or 51, wherein at least 70% of said microparticles having a volumetric mean diameter from about 0.01 μm to about 2.5 μm.

53. The pharmaceutical composition according to any of claims 50-52, wherein at least 90% of said microparticles having a volumetric mean diameter from about 0.01 μm to about 2.5 μm.

54. A method of treating, managing or lessening the severity of symptoms and infections associated with one or more pulmonary diseases or infections in a subject, the method comprising administering to the subject a bismuth-thiol (BT) composition that comprises amorphous BisEDT according to nay of claims 1-10, wherein the BT composition comprises a plurality of microparticles wherein at least 70% of said microparticles having a volumetric mean diameter from about 0.01 μm to about 2.5 μm, and wherein when the BT composition is aerosolized, at least 70% of the aerosolized liquid droplets have a MMAD from about 0.03 μm to about 3 μm.

55. The method of claim 54, wherein the one or more pulmonary diseases or infections are not the result of or associated with cystic fibrosis.

56. The method of claim 54 or 55, wherein the pulmonary infection is bronchiectasis infection, pneumonia, valley fever, allergic bronchopulmonary aspergillosis (ABPA), ventilator acquired pneumonia, hospital acquired pneumonia, community acquired pneumonia, ventilator associated tracheobronchitis, lower respiratory tract infection, non-tuberculous Mycobacteria (NTM), Mycobacterium tuberculosis, anthrax, legionellosis, pertussis, bronchitis, Bronchiolitis, COPD-associated infection, and post-lung transplantation.

57. The method of claim 56, wherein the pulmonary infection is non-tuberculous Mycobacteria (NTM).

58. A method for healing a wound in a subject having a diabetic foot infection, comprising administering the subject a therapeutically effective amount of a composition comprising amorphous BisEDT according to any one of claims 1-10, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is healed or substantially healed within 12 weeks of the first administration of the composition.

59. The method of claim 58, wherein the wound is a diabetic foot ulcer.

60. The method of claim 16-17 or 58 or 59, wherein the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

61. The method of any one of claims 16-17 or 58-60, wherein the applied BT composition is present on the surface at a concentration from about 1 μg/cm² to about 1,000,000 μg/cm².

62. The method of any one of claims 16-17 or 58-61, wherein the applied BT composition is present on the natural surface at a concentration from about 50 μg/cm² to about 100 μg/cm².

63. The method of any one of claims 16-17 or 58-62, wherein the applied BT composition is present on the surface at a concentration greater than about 100 μg/cm².

64. The method of any one of claims 16-17 or 58-63, wherein the BT composition is administered three times per day, two times per day, once daily, every other day, once every three days, three times per week, once every week, once every other week, once every month, or once every other month.

65. The method of any one of claims 16-17 or 58-64, wherein the wound is healed 4 weeks, 8 weeks or 12 weeks after the first administration of the BT composition.

66. The method of any one of claims 16-17 or 58-65, wherein the subject is administered multiple doses of the BT composition daily or weekly for a length of time ranging from about one week to about 12 weeks.

67. The method of any one of claims 16-17 or 58-66, wherein the subject is administered multiple doses of the BT composition daily or weekly for a length of about 4 weeks.

68. The method of any one of claims 16-17 or 58-67, wherein the wound area is from about 0.1 cm² to about 250 cm².

69. A method for wound size reduction in a subject having a diabetic foot infection, comprising administering to the subject a therapeutically effective amount of a composition comprising amorphous BisEDT according to claims 1-10, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is reduced in size from about a 1% reduction relative to the original wound size to total elimination of the wound within 12 weeks of the first administration of the composition.

70. The method of any one of claims 16-17 or 69, wherein the wound is reduced by about 10%, 20%, 30%, 40%, 50%, 60%, 70%, 80%, or 90%.

71. The method of claim 16-17 or 69 or 70, wherein the wound is reduced by at least about 50%.

72. The method of any one of claims 16-17 or 69-71, wherein the wound is a diabetic foot ulcer.

73. The method of any one of claims 16-17 or 69-72, wherein the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

74. The method of any one of claims 16-17 or 69-73, wherein the applied BT composition is present on the surface at a concentration from about 1 μg/cm² to about 1,000,000 μg/cm².

75. The method of any one of claims 16-17 or 69-74, wherein the applied BT composition is present on the surface at a concentration from about 50 μg/cm² to about 100 μg/cm².

76. The method of any one of claims 16-17 or 69-75, wherein the applied BT composition is present on the surface at a concentration greater than about 100 μg/cm².

77. The method of any one of claims 16-17 or 69-76, wherein the BT composition is administered three times per day, two times per day, once daily, every other day, once every three days, three times per week, once every week, once every other week, once every month, or once every other month.

78. The method of any one of claims 16-17 or 69-77 wherein the BT composition is administered once daily or three times per week.

79. The method of any one of claims 16-17 or 69-78, wherein the subject is administered multiple doses of the BT composition daily or weekly for a length of time ranging from about one week to about 12 weeks.

80. The method of any one of claims 16-17 or 69-79, wherein the subject is administered multiple doses of the BT composition daily or weekly for a length of about 4 weeks.

81. The method of any one of claims 16-17 or 69-80, wherein the wound area is from about 0.1 cm² to about 250 cm².

82. The method of any one of claims 16-17 or 69-81, wherein the wound surface area of said wound is reduced by at least 50% by 12 weeks after the first administration of the BT composition.

83. The method of any one of claims 16-17 or 69-82, wherein the wound surface area of said wound is reduced by at least 50% by 4 weeks after the first administration of the BisEDT composition.

84. The method of any one of claims 16-17 or 69-83, wherein the wound surface area is measured using digital photographs or hand measurement.

85. A pharmaceutical composition comprising bismuth-thiol (BT) composition that comprises amorphous BisEDT according to any one of claims 1-10 suspended therein, wherein the BT composition comprises a plurality of microparticles, wherein the D90 of said microparticles is less than or equal to 1.9 μm.

86. The pharmaceutical composition of claim 85, wherein the BT composition comprises a plurality of microparticles, wherein the D90 of said microparticles is less than or equal to about 1.6 μm.

87. A method for healing a wound in a subject having a diabetic foot infection, comprising administering the subject a therapeutically effective amount of a composition of claim 85 or claim 86.
